(19) United States
(12) Patent Application Publication
(10) Pub. No.: US 2026/0085287 A1
(43) Pub. Date: Mar. 26, 2026

(54) THERAPEUTIC MACROPHAGES

(71) Applicant: Resolution Therapeutics Limited, London (GB)

(72) Inventors: Lara CAMPANA, London (GB); Kayleigh THIRLWELL, London (GB); Niya EKSANDROVA ALEKSIEVA, London (GB); Rafael Alejandro ARMESILLA DIAZ, London (GB); Hollie BARTLEY, London (GB)

(73) Assignee: Resolution Therapeutics Limited, London (GB)

(21) Appl. No.: 19/112,932

(22) PCT Filed: Sep. 27, 2023
(86) PCT No.: PCT/EP2023/076752

§ 371 Date: Mar. 18, 2025

(30) Foreign Application Priority Data
Sep. 27, 2022 (GB) 2214084.2
Sep. 27, 2022 (GB) 2214139.4

(51) Int. Cl. C12N 5/0786; A61K 35/15; A61K 40/17; A61K 40/50; A61P 1/16; C07K 14/54; C12N 5/00; C12N 5/10; C12N 9/64; C12N 15/85

(52) U.S. Cl. CPC C12N 5/0645; A61K 35/15; A61K 40/17; A61K 40/50; A61P 1/16; C07K 14/5428; C12N 5/10; C12N 5/562; C12N 9/6489; C12N 15/85; C12Y 304/24035; C12N 2501/22; C12N 2501/2304; C12N 2501/2313; C12N 2510/00

(57) ABSTRACT

The present invention relates to a macrophage, genetically engineered to overexpress Interleukin-10 (IL-10) or IL-10 in combination with Matrix Metallopeptidase 9 (MMP9). Such a macrophage may be for use in treatment of an inflammatory condition in a subject such as inflammatory organ damage. The inflammatory condition may be acute or chronic and may involve a fibrotic element.

### FIELD OF THE INVENTION

The present invention relates to a macrophage, genetically engineered to express a combination of Interleukin-10 (IL-10) and Matrix Metallopeptidase 9 (MMP9). The macrophage is preferably engineered with exogenous nucleic acids encoding IL-10 and MMP9, which may, in some instances, be expressed from the same nucleic acid molecule. Such a macrophage may be for use in treatment of an inflammatory condition in a subject such as inflammatory organ damage. The inflammatory condition may be acute or chronic and may involve a fibrotic element. The present invention further relates to populations of, compositions comprising, and methods using, such macrophages or populations thereof. The present invention further relates to a method of engineering such macrophages e.g. comprising transient transfection with a combined IL-10 and MMP9 mRNA construct.

### BACKGROUND

Fibrosis is the final common pathway of chronic disease of various aetiologies, including toxic damage, viral infections, metabolic and genetic diseases, and autoimmune diseases. Acute, self-limiting fibrosis has likely evolved as a reversible and protective response to injury. The balance between self-limited and excessive fibrosis is finely regulated by multiple pathways and systems, and essentially dependent on the duration and repetition of the injury. A paradigm for the biology of fibrosis generation and remodelling is offered by the liver. End-stage, chronic liver fibrosis also known as cirrhosis, is a life-threatening condition^{1,2}. Mortality due to liver disease is the only leading cause of death that has steadily increased year on year since the 1970s in the United Kingdom and it remains a major health burden worldwide³. Hepatic decompensation (HD), defined by the acute development of one or more major complications of cirrhosis (i.e., ascites, encephalopathy, gastrointestinal variceal haemorrhage, and spontaneous bacterial peritonitis), represents a morbid advancement during the clinical course of liver cirrhosis [Trebicka 2020⁴³] and is the most common cause of hospitalisation in patients with liver cirrhosis [Moreau 2013⁴⁶]. Patients with HD are at high risk for short-term death [Moreau 2013^{46].} The first episode of HD (also referred to herein as a first hepatic decompensation event), which often requires hospitalisation, signals the transition from compensated to decompensated cirrhosis. Decompensated cirrhosis is characterised by recurrent episodes of HD⁴². HD has two distinct clinical presentations, depending on the presence or absence of other organ failures and the grade of systemic inflammation. The presence of multiple organ failures and high-grade systemic inflammation is the hallmark of acute-on-chronic liver failure (ACLF), a syndrome associated with a very high 28-day mortality rate. HD associated with moderate systemic inflammation not involving additional organs has a lower 28-day mortality rate (˜2%, although this increases to 10% at 90-days suggesting a heterogeneity of clinical course in patients with HD) [Trebicka 2020⁴³] but still portends a poor outcome over the ensuing years.

The only therapeutic approaches for liver injury entail removal of the injurious stimuli (e.g. administration of an efficacious anti-viral therapy) and liver transplantation. Treatment of liver failure arising from either acute or chronic injury is limited to supportive care and/or transplantation, the latter of which requires donors. Currently there is a deficit of available organ donors and the surgical procedure carries significant morbidity and mortality risks. In addition, patients are also committed to life-long immunosuppression. Furthermore, despite the relative success of therapeutic interventions for specific aetiologies (e.g., novel antiviral therapy for hepatitis C virus infection, alcohol abstinence for alcoholic liver disease), many diseases (e.g. NASH) have not got approved medical therapies and patients often present to medical attention late when cirrhosis and related complications have already occurred [Starkey 2019⁴⁴]. Thus, there are no specific therapies to treat hepatic cirrhosis and so delivering an effective anti-fibrotic therapy is therefore a major unmet clinical need for both chronic and acute liver damage⁴⁻⁶.

Macrophages (Mφ) play a pivotal role in the inflammatory response in the injured liver. In the liver there are two main populations of Mφ: (i) resident macrophages (Kupffer Cells, KCs), and (ii) infiltrating macrophages. KCs carry out patrolling functions in the liver sinusoids to phagocytose microbial debris that reach the liver via the sinusoidal capillaries in homeostatic conditions. During the early phases of liver damage, KCs express chemokines such as CCL2 and CCL5, thereby contributing to the recruitment of monocytes from the circulation^{2,7}. The number of KCs decreases during fibrosis; they then repopulate the liver in the recovery phase of self-limiting fibrosis⁸. Infiltrating monocyte derived Mφ (MDMs) play a major role in the response to liver damage. Infiltrating Mφ are recruited via the CCR2/CCL2 axis; once in the liver parenchyma, they locate along the fibrotic septa in the early stages of liver fibrosis and may promote fibrosis by releasing factors such as TGF-β, IL1, PDGF and CCL2 that activate hepatic stellate cells and worsen inflammation. This may suggest a detrimental role for Mφ in progressive fibrosis. However, if Mφ are depleted at the onset of fibrosis remodelling, the remodelling process fails, and the liver fibrosis persists. It is now widely accepted that macrophages play a dual role in the establishment and resolution of fibrosis^{2,8-11}.

Due to the suggested roles of macrophages in healing of fibrosis, it has been considered that a macrophage cell therapy could be beneficial for the reduction of chronic liver fibrosis. It has been shown that mouse bone marrow-derived macrophages (BMDMs) decrease liver fibrosis when injected into a mouse model of chronic liver fibrosis¹². Similar results have been replicated using human monocyte-derived macrophages (hMDMs) in immunodeficient mouse models of chronic liver fibrosis¹³. Furthermore, a GMP-graded cell culture protocol¹⁴ is currently used to generate hMDMs for autologous transplantation into cirrhotic patients in an ongoing phase II trial (MATCH, Macrophage therapy for liver disease, ISRCTN10368050, EudraCT reference 2015-000963-15). Macrophages are also pivotal in solving acute conditions³⁸. However, to date there exists no therapy (either macrophage cell therapy or otherwise) which can efficiently treat patients suffering from liver cirrhosis by acting through 4 crucial mechanisms—recruitment of cells of the immune system to the liver (paracrine effect), conversion of recruited monocytes into pro-restorative macrophages which are effective in propagating the treatment effect (polarisation), phagocytosis and remodelling of fibrotic scar. In particular, to date there exists no therapy which is able act through all these mechanisms efficiently enough to enable treatment of patients suffering from decompensated liver cirrhosis and that have undergone a hepatic decompensation event.

### INTRODUCTION TO THE INVENTION

Typically, human macrophages derived in vitro from monocytes (hMDM) will express IL-10, but this is not secreted from the macrophage in significant quantities as shown in the results presented in FIG. 1. Herein, the inventors enhance macrophage anti-fibrotic and anti-inflammatory properties thanks to the expression and secretion of IL-10 and MMP9 by means of transient transfection, as a proof of principle of how the additional expression of these genes delivers a promising product to help inflammatory conditions such as acute and chronic organ damage resolution, delivering macrophages that secrete IL-10 and active MMPs.

IL-10 is known for its anti-inflammatory properties, whilst some MMPs have a role in helping fibrosis remodelling. However, the effects of the additional expression of IL-10 and MMP9 in combination are currently unknown. Additionally, the inventors demonstrate that macrophages engineered to overexpress IL-10 can be used in effective therapies, in particular for liver cirrhosis. In particular, without being bound by theory or mechanism, the engineered macrophages described herein, and in particular engineered macrophages that express both IL-10 and MMP9 demonstrate activity and/or improvement in each of 4 mechanisms of action that are thought to be the basis for the efficacy of therapeutic macrophages, namely: 1) Remodelling of fibrotic scar; 2) Phagocytosis; 3) Paracrine Effect; and 4) Polarisation. FIG. 32, demonstrates how these mechanisms of actions contribute to the therapeutic effect of the macrophages, and in particular how recruitment of endogenous monocytes and their polarisation into pro-restorative macrophages is able to amplify the therapeutic effect (this amplification also termed the “virtuous cycle”).

We show that human monocyte derived macrophages (hMDMs) transfected (Trx) with an IL-10 or IL-10-p2A-MMP9 (herein referred to as IL-10+MMP9) expressing mRNA can polarise macrophages towards a pro-restorative phenotype, based on the expression of CD14, CD206high, 25F9, CD163, CCR2low/neg, CD86low and HLA-Class II low. Furthermore, the inventors demonstrate in the Examples that IL-10 Trx and IL-10-MMP9 Trx hMDMs can recruit significantly more monocytes as compared to the hMDMs currently used in the MATCH clinical trial (Trial registration numbers: ISRCTN10368050 and EudraCT; reference 2015-000963-15. Study protocol: a multicentre, open-label, parallel-group, phase 2, randomised controlled trial of autologous macrophage therapy for liver cirrhosis) when they are tested in a peripheral blood mononuclear cell (PBMC) migration assay. Recruitment may be tested in any suitable way, such as techniques based on a Boyden chamber. In the Examples, the inventors test immune cell migration in a transwell cell migration and invasion assay (Boyden chamber assay). Without wishing to be bound by theory or mechanism, IL-10-Trx macrophages are able to specifically recruit monocytes, without substantially recruiting other immune cell types. Surprisingly, IL-10-MMP9 Trx hMDMs have a superior ability to recruit monocytes as compared to MMP9-Trx and IL-10-Trx. The two proteins appear to show a synergistic effect in this this process. Indeed, MMP9 only transfected macrophages have been shown to have little or no ability to attract monocytes (FIG. 12), whilst the activity of matrix metalloproteases in general sees an increase in such cells (data not shown). Further, IL-10 and MMP9 Trx hMDMs show excellent anti-inflammatory profile and phagocytic capacity.

Finally, the inventors discovered that overexpression of IL-10 alone reduced the overall matrix metalloprotease (MMP) activity and the level of MMP9 secreted in the supernatant of the genetically engineered macrophages. Metalloprotease assays to determine levels of activity are as described in the Examples. The negative effect of IL-10 on MMP activity is in line with findings in the literature (for example: Roth et al., IL-10 Is an Autocrine Inhibitor of Human Placental Cytotrophoblast MMP-9 Production and Invasion, Developmental Biology, Volume 205, Issue 1, 1999, Pages 194-204; and Krishnamurthy et al., IL-10 inhibits inflammation and attenuates left ventricular remodeling after myocardial infarction via activation of STAT3 and suppression of HuR, Circ Res., 2009 Jan. 30; 104(2)).

The inventors, therefore, also provide a macrophage engineered with a sequence encoding a specific MMP, MMP9. Interestingly, whilst MMP9 expression does not increase dramatically when comparing non-Trx and IL-10+MMP9 Trx hMDMs, the overall matrix metalloproteases activity of IL-10+MMP9 Trx hMDMs increases significantly.

Taken together, these data support the use of IL-10+MMP9 Trx hMDMs as a therapeutic product in the treatment and/or prevention of inflammatory conditions (e.g. related to organ damage). Despite IL-10 being known as an anti-inflammatory cytokine, this is somewhat surprising, as previous data showed that IL-4-stimulated, but not IL-10 stimulated macrophages, were pro-restorative in animal models of acute liver disease¹⁵. A combination of additional IL-10 and MMP9 expression (e.g., via transfection) may also be envisaged to reinforce the anti-fibrotic (via matrix metalloprotease activity increase) and pro-restorative (via increase in monocyte migration) function of macrophage-based therapeutic products. The recruitment of monocytes to the site of inflammation by such IL-10 and MMP9 expressing macrophages is critical, since the macrophages of the invention are thus recruiting monocytes in situ to provide further therapeutic effect. Recruited monocytes may be converted to pro-restorative macrophages, thus amplifying the effect of providing therapeutic macrophages. A pro-restorative phenotype is described extensively herein.

Chronic inflammatory organ damage is often associated with fibrosis, such as for example in chronic liver disease. Therefore, a pro-restorative therapeutic macrophage with anti-inflammatory and anti-fibrotic functions would be beneficial.

As mentioned above, the inventors have found that macrophages engineered to overexpress IL-10 exhibit a depression of overall MMP activity. In contrast, macrophages engineered to additionally express MMP9, for example by engineering with a nucleic acid encoding MMP9, exhibit an enhancement in overall MMP activity, not just MMP9 activity. Herein, for the first time, by combined expression of IL-10 and MMP9, the inventors enhanced the overall MMP activity—of the engineered macrophage to a greater degree than would be expected from the data of macrophages separately transfected with IL-10 or MMP9 alone. Hence the inventors demonstrate a surprising synergistic effect with the combined expression of IL-10 and MMP9 on overall MMP activity. Thus, the macrophage is engineered such that it is provided with an additional/exogenous coding sequence for IL-10 and MMP9 beyond those present naturally in the cell. Such a combination is not taught nor suggested in the prior art. We show that macrophages transfected with a combined mRNA construct expressing IL-10 and MMP9 may be polarised towards macrophages with aspects of a pro-restorative phenotype, as described herein.

Furthermore, the inventors demonstrate that IL-10-MMP9-Trx hMDMs locate to the liver in a disease model of liver inflammation/fibrosis. Also demonstrated is the ability of the engineered macrophages to recruit significantly more monocytes as compared to the hMDMs currently used in MATCH, which are not genetically engineered. Such recruitment studies were performed in vitro, using standard assays as described herein. Taken together, these data support the use of IL-10 and MMP9-Trx hMDMs as an improved therapeutic product in the treatment and/or prevention of inflammatory conditions e.g. related to organ damage.

Matrix metalloproteases (MMPs), also known as matrix metalloproteinases or matrixins, are a family of zinc-dependent polypeptidases that collectively degrade various proteins in the extracellular matrix (ECM). MMPs have a magnitude of roles in vivo, making them extremely complex to understand. In the context of fibrosis, it is reported that MMPs are contributory, however, they are also pivotal in fibrosis regression through their matrix remodelling capacity. Their dual role in fibrosis progression and regression can somewhat be appreciated through their complex in vivo activity ranging from cytokine and chemokine activation, immune cell recruitment and activation to extracellular matrix degradation. In addition, the activation and subsequent activity of MMPs in vivo is highly regulated, largely by tissue inhibitors of metalloproteinases (TIMPS), adding to the difficulty of understanding their activity in fibrosis. Experimental models of lung fibrosis have exhibited an increase in MMP9 (a type IV collagenase targeting collagen) activity which has also been associated with the disruption of the alveolar epithelial membrane, indicating a putative profibrotic role of MMP9 in lung injury. However, in a bleomycin-induced model of lung fibrosis, MMP9 null mice develop similar lung fibrosis to wild-type littermates, although the lungs of the MMP9 deficient mice showed limited alveolar bronchiolisation. Overall, the precise role of MMPs in fibrosis is not completely understood. Moreover, in vitro derived hMDMs express negligible levels of MMPs; plus, hardly any activity is detected in the conditioned media of these cells.

Little has been reported thus far in the field of MMP genetic engineering. MMP9 transfection in THP1 cells has been utilised as a research tool to understand the inflammatory responses of macrophages in atherosclerosis. Therapeutically, the overexpression of MMP9 in iPSC via lentiviral transduction has been utilised to provide enhanced repair to damaged myocardium. MMP12 (an elastase targeting soluble and insoluble elastin) has been overexpressed in endothelial progenitor cells for use in a melanoma cell therapy. None of the previous work performed with MMP transfection of human macrophages, as far as the present inventors are aware, has resulted in macrophages with anti-fibrotic properties. Cabrera et al. demonstrated overexpression of MMP9 in macrophages in transgenic mice challenged with bleomycin resulted in attenuation of fibrosis⁴⁵. Differences in the levels of TIMP-1 between the transgenic mice and wild-type mice were also observed. However, a difference in expression levels or activity of other matrix metalloproteases was not reported.

MMPs have a complex biology, which often is organ and disease specific. For example, MMP9 has a marked anti-fibrotic effect in models of chronic liver disease and chronic lung disease. However, it seems to be detrimental in kidney fibrosis.

WO2019/118888A1 (TREATMENT OF FIBROSIS WITH GENETICALLY-ENGINEERED MACROPHAGES) describes macrophages engineered for treating fibrosis. For example, a genetically-engineered macrophage, comprising: a recombinant extracellular matrix (ECM) targeting protein; and/or a recombinant protease. The recombinant protease may be a matrix metalloproteinase (MMP), e.g. one from a long list of possible MMPs including MMP9 and MMP12. But there is no mention of IL-10 or cytokines in relation to the engineering of the cells.

WO2022/047119A1 (MODIFIED IMMUNE CELLS FOR FIBROSIS AND INFLAMMATION) describes a modified immune cell comprising one or more nucleic acid sequences encoding: (i) at least one exogenous fibrolytic agent, and/or (ii) at least one exogenous anti-inflammatory agent. The at least one exogenous fibrolytic agent may comprise a matrix metallopeptidase (MMP), e.g. one or more from a long list of possible MMPs including MMP9 and MMP12. The at least one exogenous anti-inflammatory agent may comprise e.g. a cytokine, a chemokine, or a pentraxin, and the cytokine may comprise e.g. IL-10, IL-4, IL-13, and/or TGF-beta. The modified immune cell may comprise a macrophage. However, no specific combinations of these possible genes and cell types are disclosed, and furthermore no combinations are exemplified in this application—single genes only are tested. Moreover, the applicants do not seek to determine the effect of, for example, IL-10 expression on the expression of other proteins, such as the MMPs. Further, the data provided in the WO2022/047119A1 application indicates that the macrophages transfected with IL-10 raise at least one pro-inflammatory marker, CD-80, on the macrophage, which is not desirable for treating inflammatory conditions.

WO2019/175595 described use of autologous isolated non-engineered human macrophages in the treatment of liver disease, including liver cirrhosis. However, the macrophages in this application are non-polarised and non-engineered. Accordingly, such cells are less useful for treating inflammatory conditions and those with a fibrotic element.

WO2012/062930 describes a use of a composition comprising a macrophage overexpressing IL-10 from transfected mRNA as a medicament. However, the application does not disclose combination of IL-10 and MMP9 or treatment of liver cirrhosis.

The present inventors have demonstrated for the first time the advantageous effects of macrophages engineered to express the specific combination of IL-10 and MMP9, for example in the treatment of inflammatory and fibrotic diseases.

One or more aspects or embodiments of the claimed invention aim to solve one or more of the above-mentioned problems.

### STATEMENTS OF INVENTION

According to a first aspect of the invention, there is provided an engineered macrophage engineered to overexpress IL-10. In certain embodiments, the macrophage secretes IL-10 at a culture supernatant concentration of at least 10,000 pg/ml when cultured in vitro at a cell concentration of 4×10⁶/ml. As described herein, engineered macrophages overexpressing IL-10 alone demonstrate a significant ability to recruit monocytes, anti-inflammatory secretome and to convert unpolarised or pro-inflammatory macrophages to a pro-restorative phenotype. The examples demonstrate that such a macrophage is useful in therapy, in particular for treating liver cirrhosis.

According to another aspect of the invention, there is provided an engineered macrophage that overexpresses IL-10 and MMP9, such that the engineered macrophages express greater levels of IL-10 and MMP9 than untransfected macrophages. According to an aspect of the present invention, there is provided an engineered macrophage, wherein the macrophage is engineered to express IL-10 and MMP9, preferably by the provision of additional or exogenous sequences encoding these proteins. Such IL-10 and MMP9 engineered macrophages may have utility in therapy, for example, for use in treating an inflammatory condition and/or a fibrotic condition in a subject.

In other aspects of the invention are provided engineered macrophages which are engineered to overexpress IL-10 and IL-4; IL-10 and MMP12; IL-4, IL-13 and MMP9; or IL-4, IL-13 and MMP12.

Engineered macrophages according to any aspect of the invention may have particular structural and advantageous functional properties, as described herein.

Exemplary structural and functional properties of the engineered macrophages are set out in Table 4, relative to non-polarised, non-transfected cells used in the MATCH study described in WO2019175595. In some embodiments, the engineered macrophages of the invention exhibit any combination of the properties described in Table 4.

Further structural and functional properties of the engineered macrophages are set out in Table 5. In some embodiments, the engineered macrophages of the invention exhibit any combination of the properties described in Table 5. According to some embodiments, the engineered macrophages of the invention exhibit at least all of the properties described in Table 5.

In preferred embodiments, when exposed to non-engineered macrophages, the engineered macrophages polarise non-engineered macrophages to a pro-restorative phenotype. When administered to a subject, the engineered macrophages may polarise host macrophages (such as endogenous monocyte derived macrophages that migrate into the liver) to a pro-restorative phenotype. In some embodiments, the engineered macrophage may convert unpolarised host macrophages to a pro-restorative macrophage. In some embodiments, the engineered macrophage may convert pro-inflammatory host macrophages to a pro-restorative macrophage. In some embodiments, conditioned medium from engineered macrophages may convert non-engineered macrophages to a pro-restorative phenotype. Accordingly, the engineered macrophages of the invention may convert non-engineered macrophages to a pro-restorative phenotype in vitro or in vivo. Conversion to a pro-restorative phenotype may increase the expression of CD206 and CD163, and decrease the expression of CD86 and HLA-DR on the cell surface.

In some embodiments the engineered macrophage has an at least two-fold reduced expression of CD86 compared to non-engineered, non-polarised cells. In some embodiments the engineered macrophage has an at least two-fold reduced expression of HLA-DR compared to non-engineered, non-polarised cells. In some embodiments the engineered macrophage has an at least 1000-fold increased secretion of IL-10 compared to non-engineered, non-polarised cells. In some embodiments the engineered macrophage has an at least 10-fold increased secretion of MMP3 compared to non-engineered, non-polarised cells. In some embodiments the engineered macrophage has an at least 20-fold increased secretion of MMP10 compared to non-engineered, non-polarised cells.

In some embodiments the macrophage of the invention secretes IL-10 at a culture supernatant concentration of at least 10,000 pg/ml when cultured in vitro at a cell concentration of 4×10⁶/ml. In some embodiments the macrophage of the invention secretes MMP9 at a culture supernatant concentration of at least 200 ng/ml when cultured in vitro at a cell concentration of 4×10⁶/ml. In some embodiments the engineered macrophage has an at least 5-fold increased expression of CD206 compared to monocytes. In some embodiments engineered macrophage has an at least 5-fold increased expression of 25F9 compared to monocytes. In some embodiments the engineered macrophage has an at least ten-fold reduced expression of CD80 compared to non-engineered cells. In some embodiments the macrophage of the invention secretes TNF-α at a culture supernatant concentration of at least 40 pg/ml when cultured in vitro at a cell concentration of 4×10⁶/ml. In some embodiments the engineered macrophage has the same phagocytic ability as a non-engineered, non-polarised cell.

In one embodiment, the present invention provides an engineered macrophage comprising one or more exogenous coding sequences for IL-10 and MMP. Said exogenous coding sequence may be any suitable nucleic acid sequence. Said exogenous coding sequence may be present in the cytoplasm or nucleus as an extrachromosomal nucleic acid or integrated into the macrophage genome. Said exogenous coding sequence may encode IL-10 and MMP9, or a plurality of exogenous coding sequences may each encode IL-10 or MMP9.

In some embodiments, endogenous IL-10 and/or MMP9 gene expression can be stimulated by genetic engineering. For example, gene editing techniques such as CRISPR can be used to turn on and off the endogenous genes that encode IL-10 and or MMP9, generating an engineered macrophage that expresses IL-10 and/or MMP9 under conditions in which it would not otherwise express these proteins. This may be done by altering the promoter sequence, for example.

Natural non-engineered macrophages are capable of expressing IL-10 and/or MMP9 under relevant physiological conditions, but generally natural macrophages do not secrete significant levels of IL-10. However, the present invention does not relate to these natural non-engineered macrophages, but instead to engineered macrophages wherein the expression level of IL-10 in particular has been raised to supra-physiological levels, thereby improving the anti-inflammatory properties of the therapeutic macrophages. As used herein, the inventors describe this as “overexpression” of IL-10. In order to overexpress the IL-10, the macrophage may be engineered such that it possesses additional or exogenous coding sequences for IL-10. In some embodiments, the cell is transfected with mRNA encoding IL-10/MMP9.

A cell which overexpresses IL-10 and/or MMP9 comprises coding sequences expressing IL-10 and/or MMP9 at a higher level than in non-engineered cells. As described above, overexpression may be achieved through the introduction of exogenous nucleic acid encoding IL-10 and/or MMP9 such as mRNA, or genetic modification which stimulates expression of IL-10 and/or MMP9 from endogenous coding sequences. An engineered macrophage which overexpresses IL-10 and/or MMP9 may not necessarily secrete a greater amount of IL-10 and/or MMP9 than a non-engineered macrophage.

In other embodiments, the macrophage may be engineered to turn on endogenous genes that encode IL-10 and/or MMP9. In any case, the macrophages of the disclosure have been modified through alterations in IL-10 and/or MMP9 expression levels by any means and thus are referred to as engineered macrophages.

In over-expressing IL-10, the natural activity levels of the MMPs, including MMP9, fall.

Therefore, the engineered macrophage is provided with an additional or exogenous MMP coding sequence to maintain at least “physiological” levels of MMP9 expression, or levels just above physiological. In some embodiments, MMP9 expression is modified to rescue, restore or return the macrophages into expressing levels of MMP9 comparable or increased when compared to macrophages not transfected with IL-10. Thus, despite the fact that the engineered macrophage is engineered with additional/exogenous MMP9 coding sequence, the engineered macrophage demonstrates MMP9 expression levels similar or slightly above wild-type/natural expression. Slightly above may mean an enhancement of expression over natural expression levels of between 1.2 and 1.5 times, such as 1.2, 1.3, 1.4 or 1.5 times the natural level.

The macrophage may be engineered to express both IL-10 and MMP9. This expression may be driven from the endogenous genes in some embodiments. In other embodiments, the macrophage is engineered to contain exogenous coding sequence(s) for IL-10 and MMP9. It may be preferred that the macrophage over-expresses IL-10. It may be preferred that the macrophage over-expresses MMP9. Alternatively, the macrophage may be engineered such that there is over-expression of both IL-10 and MMP-9. Expression levels of IL-10 and/or MMP9 are increased when compared to a non-transfected macrophage. Expression levels of MMP9 are increased when compared to a macrophage transfected with IL-10 alone.

In some embodiments, the macrophage that is engineered is a human monocyte-derived macrophage. In some embodiments, the macrophage is derived from monocytes by culturing in the presence of MCSF. Suitable culturing conditions are discussed further below

In some embodiments, the baseline macrophages (i.e., pre-engineered or natural) are referred to as unpolarised human monocyte-derived macrophages, also terms “resting” macrophages.

In some embodiments, the macrophage that is engineered is a macrophage derived from an iPSC. There are various methods known in the art to derive macrophages from iPSC. At a baseline, these would also be unpolarised or resting. Accordingly, in certain embodiments, the engineered macrophage is derived from a pluripotent stem cell that is cultured in vitro. Preferably, such pluripotent stem cell-derived or iPSC-derived macrophages are hypoimmunogenic due to the knock out or knock down of at least one gene associated with either HLA Class I and/or HLA class II cell surface molecules. For example, the gene associated with HLA Class I may be HLA-A, HLA-B and HLA-C genes or B2M gene. The gene associated with HLA Class II may be HLA-DP, DM, DO, DQ, and DR or CIITA. This knock-out or knock-down is evident in the macrophages, but may be introduced into any progenitor cell as the macrophage is differentiated from the stem cell. In exemplary methods, the engineered macrophages of the invention may be generated by providing a stem cell, such as an induced pluripotent stem cell, optionally knocking out or down expression of at least one sequence encoding a unit in the HLA I or HLA II complex, differentiating the stem cells into embryoid bodies, optionally using BMP4, SCF, VEGF and/or Rock Inhibitor (Y-27632), differentiating the embryoid bodies into macrophage progenitors, optionally using M-CSF and IL-3, and maturing the macrophage progenitors into functional macrophages, optionally using M-CSF.

As used herein, over-expression relates to the artificial expression of a gene in increased quantity.

As used in the Examples, expression levels of the proteins were quantified at between 16 to 24 hours post-transfection. Expression levels as recited herein are given for a population of macrophages at a concentration of 4×10⁶/ml (which equates to 2×10⁶ cells per cm²). In the Examples, the macrophages were transfected, isolated by centrifugation, re-suspended in TexMACs buffer supplemented with IL-3 and IL-14, and incubated at 37° C. under 5% CO₂. Those skilled in the art would be aware of equivalent conditions suitable to determine secreted protein concentration.

In some embodiments, the macrophage is engineered to overexpress IL-10, wherein the secreted IL-10 protein level is greater than about 300 pg/ml. Suitably, the secreted IL-10 protein is greater than about: 300 pg/ml or 400 pg/ml or 500 pg/ml or 600 pg/ml or 700 pg/ml or 800 pg/ml or 900 pg/ml or 1,000 pg/ml or 2,000 pg/ml or 3,000 pg/ml or 4,000 pg/ml or 5,000 pg/ml or 6,000 pg/ml or 7,000 pg/ml or 8,000 pg/ml or 9,000 pg/ml or 10,000 pg/ml or 11,000 pg/ml. Suitably, these IL-10 protein levels may be measured by culturing the macrophages as described above, wherein the concentration of macrophages in the medium is 4×10⁶ cells/ml, which equates to 2×10⁶ cells/cm², and measuring the concentration of the protein in the culture medium. In preferred embodiments, the macrophage secretes IL-10 at a culture supernatant concentration of at least 10,000 pg/ml, 15,000 pg/ml or 20,000 pg/ml, when cultured in vitro at a cell concentration of 4×10⁶/ml. In a particularly preferred embodiment, the IL-10 is secreted at a culture supernatant concentration of 49,000 μg/ml or greater. In some embodiments, the engineered macrophage may secrete IL-10 at levels 1000 fold greater than non-engineered, non-polarised hMDMs. Non-engineered, non-polarised hMDMs are described in the art, for example in WO2019175595.

In some embodiments, the macrophage is engineered to express MMP9, wherein the secreted MMP9 protein level is greater than about 200 ng/ml. Suitably, the secreted MMP9 protein is greater than about: 300 ng/ml or 400 ng/ml or 500 ng/ml or 600 ng/ml or 700 ng/ml or 800 ng/ml or 900 ng/ml or 1,000 ng/ml. Suitably the secreted MMP9 protein level is between about 200 ng/ml and 2000 ng/ml. In preferred embodiments, the secreted MMP9 protein level is greater than 200 ng/ml. In one embodiment, the secreted MMP9 protein level is greater than 1500 ng/ml. Suitably the engineered macrophage (comprising IL-10 and MMP9) has a secreted MMP9 protein level that is greater than the average level of secreted MMP9 protein in macrophages engineered with IL-10 alone. In some embodiments, the engineered macrophage (comprising IL-10 and MMP9) has a secreted MMP9 protein level that is at least equal to the average level of secreted MMP9 protein in non-polarised, non-transfected macrophages. Suitably, the overall MMP activity of the engineered macrophage of the invention is also higher than the overall MMP activity of a macrophage engineered with IL-10 alone. In other embodiments, the overall MMP activity of the engineered macrophage is greater than that of an untransfected macrophage. In a preferred embodiment, the MMP activity of the engineered macrophage is at least 1.5 times greater than that of an untransfected macrophage. Suitably, these IL-10 and MMP9 protein levels may be measured—by culturing the macrophages as described above, wherein the concentration of macrophages in the medium is 4×10⁶ cells/ml, which equates to 2×10⁶ cells/cm², and measuring the concentration of the protein in the culture medium. In some embodiments, the engineered macrophage may secrete greater amounts of other matrix metalloproteases. In particular embodiments, the engineered macrophage may secrete Matrix Metalloproteinase-3 (MMP3) in 10-fold greater amounts than non-engineered, non-polarised cells. In particular embodiments, the engineered macrophage may secrete Matrix Metalloproteinase-10 (MMP10) in 10-fold greater amounts than non-engineered, non-polarised cells.

The macrophage of the present invention may be used in therapy, most notably cellular therapy, to a subject in need thereof. The subject may have a condition, disease or disorder that would benefit from the administration of the macrophages of the present invention. Such condition, disease or disorder may have an inflammatory and/or fibrotic element. Such condition, disease or disorder may be acute or chronic, or acute-on-chronic. Such condition, disease or disorder may result in organ damage. Said organ may be any suitable organ, including liver, lung or kidneys.

In some embodiments, the condition, disease or disorder in a subject is a chronic inflammatory condition with a fibrotic element. In some embodiments, the condition is chronic organ damage associated with chronic inflammation. In some embodiments, the condition is an acute inflammatory condition. In some embodiments, the condition is an acute-on-chronic inflammatory condition.

In some embodiments, the condition relates to the kidney, liver, or lung. For example, the condition maybe liver damage, kidney damage or lung damage.

In some embodiments the condition may be Acute-on-chronic liver failure (ACLF). ACLF is a syndrome characterised by acute decompensation of chronic liver disease associated with organ failures and high short-term mortality. An excessive systemic inflammatory response seems to play a crucial role in the development of ACLF.

In some embodiments, the condition may be liver injury. In preferred embodiments, the liver injury is a chronic liver injury, optionally an inflammatory liver injury. In preferred embodiments, the inflammatory liver injury has a fibrotic element. In preferred embodiments, the condition is a chronic inflammatory liver injury with a fibrotic element, preferably liver cirrhosis.

Cirrhosis represents the end-stage of chronic liver injury and progressive fibrosis (scarring), irrespective of the underlying aetiology. It is characterised by severe liver fibrosis leading to architectural disruption, hepatocyte dysfunction and portal hypertension. Various aetiologies may lead to liver cirrhosis. Hepatic disorders having a fibrotic component which may lead to fibrosis include, but are not limited to, non-alcoholic fatty liver disease (NAFL) (e.g., non-alcoholic fatty liver disease (NAFLD) or non-alcoholic steatohepatitis (NASH)) or alcoholic liver disease (e.g., alcoholic fatty liver disease (AFLD) or alcoholic steatohepatitis (ASH)).

The aetiologies leading to fibrosis may include, but are not limited to, steatotic liver disease (SLD), such as metabolic dysfunction-associated steatotic liver disease (MASLD), Metabolic-associated steatohepatitis (MASH) or Met-ALD. In some instances, the cause of fatty liver disease may be unknown, and may be termed cryptogenic SLD.

Metabolic dysfunction-associated steatotic liver disease refers to a non-alcoholic fatty liver disease, and therefore may also be known as NAFLD. Metabolic-associated steatohepatitis refers to a more severe form of MASLD, which may also be known as NASH. “Met-ALD” refers to individuals who have steatotic liver disease and who also drink alcohol. “Cryptogenic SLD” refers to SLD whose cause is unknown, such as in individuals who do not carry any known metabolic risk factors for SLD.

Fibrotic diseases, disorders, and conditions can include mechanical trauma, biliary obstruction, autoimmune hepatitis, iron overload, Hepatitis B infection (HBV), and/or Hepatitis C infection (HCV). However, the engineered macrophages according to the present invention are able to treat cirrhosis irrespective of the underlying aetiology.

Cirrhosis may be either compensated or decompensated cirrhosis (also referred to herein as hepatic decompensation or HD). Decompensated cirrhosis is defined as an acute deterioration in liver function in a patient with cirrhosis and is characterised by symptoms such as, but not limited to, jaundice, ascites, hepatic encephalopathy, hepatorenal syndrome, variceal haemorrhage, gastrointestinal haemorrhage and any combination thereof. In other embodiments, the liver disease is decompensated cirrhosis. In some embodiments, the liver disease is compensated cirrhosis. In some embodiments, the patient has compensated cirrhosis and had at least one event (or exactly one event) of decompensated cirrhosis.

In a preferred embodiment, the patient has a MELD score of between 10 and 18, or more preferably 10-16 or 12-18.

In some embodiments, the liver disease is one in which cirrhosis is caused by damage to hepatocytes, for example, is a hepatocyte-derived disease, such as those diseases of viral origin (including treated (sustained viral response) hepatitis C (HCV), hepatitis B), damage through alcoholism (alcohol related liver disease (ALD)), or non-alcoholic fatty liver disease (NAFLD), including Non-alcoholic steatohepatitis (NASH) (including NASH resulting from diabetes or obesity), cryptogenic cirrhosis, hemochromatosis or alpha-1-antitrypsin deficiency. In some embodiments, the underlying aetiology has been removed (for example, a patient suffering from damage through alcoholism is no longer drinking, or a patient suffering from damage through HCV no longer has HCV etc.). In some embodiments, the patient with liver disease is at risk of end stage renal disease.

In some embodiments, the liver disease is steatotic liver disease (SLD). In some embodiments, the steatotic liver disease is metabolic dysfunction-associated steatotic liver disease (MASLD), is Met-ALD or cryptogenic SLD. In some embodiments, the metabolic dysfunction-associated steatotic liver disease (MASLD) is Metabolic-associated steatohepatitis (MASH).

Cirrhosis may lead to acute-on-chronic liver failure (ACLF). In some embodiments, the liver disease is ACLF. ACLF is a distinct condition from hepatic decompensation. Hepatic decompensation is characterised by the development of ascites, hepatic encephalopathy, gastrointestinal haemorrhage, or any combination of these conditions in patients with liver cirrhosis. ACLF in contrast is associated with organ failures and carries high short-term mortality in excess of 15% at 28 days. Three major features characterise this syndrome: ACLF occurs in the context of intense systemic inflammation; ACLF frequently develops in close temporal relationship with pro-inflammatory precipitating events (eg infections or alcoholic hepatitis); and ACLF is associated with single- or multiple-organ failure. In some embodiments, the engineered macrophage is used in the treatment of cirrhosis in a subject with ACLF.

A diseased patient suitable for a treatment or use in accordance with any aspect or embodiment of the invention may be a patient with a relevant disease and severity.

In some embodiments, the subject has undergone their first hepatic decompensation event. In preferred embodiments, the subject has been hospitalised following their first hepatic decompensation event. The subject may exhibit one or more clinical signs of hepatic decompensation selected from the list consisting of jaundice, ascites, hepatic encephalopathy, hepatorenal syndrome, variceal haemorrhage and gastrointestinal haemorrhage. The cells, compositions and methods of the invention are expected to be particularly effective in treating patients that have been hospitalised following their first hepatic decompensation event. Furthermore, the data provided in the examples demonstrate that the cells, compositions and methods of the invention are suitable for treating these particular patients, which have severe disease that is difficult to treat.

Hospitalisation following a hepatic decompensation event is a measure of disease severity and provides a specific clinical situation. Certain symptoms of hepatic decompensation are similar to those of less severe liver cirrhosis, but when a patient is hospitalised following their first hepatic decompensation event, this indicates that their diseases is severe enough to particularly benefit from the present invention.

Accordingly, in certain embodiments, the invention provides cells and compositions for use in a method of treating a patient exhibiting one or more clinical signs of hepatic decompensation selected from the list consisting of jaundice, ascites, hepatic encephalopathy, hepatorenal syndrome, variceal haemorrhage and gastrointestinal haemorrhage, where the one or more clinical signs require hospital admission.

Hospitalisation refers to admission to a hospital for treatment. Hospitalisation thus normally requires that the patient stays in the hospital for at least 24 hours. Hospitalisation is a measure of disease severity, because symptoms cannot be managed outside of the hospital setting. Hospitalisation is a recognised measure of disease severity and patient status in the context of liver cirrhosis⁴² (see also for example, Balcar et al., United European Gastroenterol J. 2021; 9(4): 427-437.)

In preferred embodiments, the subject is treated with the cells, compositions or cell populations of the inventions once the subject has recovered from their first hepatic decompensation event (re-compensated), optionally wherein the subject was hospitalised following their first hepatic decompensation event. In some embodiments, the subject is treated with the cells, compositions or cell populations of the invention following discharge of the subject from the hospital. According to some embodiments, recovery from a hepatic decompensation event (re-compensation) is defined by a physician's clinical assessment and/or by no substantial elevation in MELD score between discharge from the hospital and treatment. In some embodiments, the subject exhibits and/or has recovered from one or more clinical signs of hepatic decompensation selected from the list consisting of jaundice, ascites, hepatic encephalopathy, hepatorenal syndrome, variceal haemorrhage, and gastrointestinal haemorrhage.

According to preferable embodiments, the subject is treated following recovery from a first hepatic decompensation event that required hospitalisation and prior to the undergoing additional hepatic decompensation events. As known in the art, the severity of decompensated cirrhosis and the mortality rate increases once a subject has had more than a single decompensation event⁴². Thus, without being bound by theory or mechanism, it is preferable to treat subjects with the cells, compositions and cell populations of the invention following recovery from the first decompensated event to increase the chances of the subjects not undergoing additional hepatic decompensation events and surviving without need for liver transplantation. In some embodiments, the macrophages that are engineered to express human IL-10 and human MMP9 may be used in the treatment of cirrhosis.

In some embodiments, the macrophages that are engineered to express human IL-10 and human MMP9 may be used in the treatment of ACLF.

In some embodiments, the macrophage has a pro-restorative phenotype and is anti-inflammatory and anti-fibrotic. Such a phenotype is defined further herein.

The macrophage engineered to express IL-10 and MMP9 may be genetically engineered in any suitable way. For example, using viral or non-viral vectors, DNA or RNA constructs or gene editing using any suitable technology. Thus, in some embodiments, the macrophage may be engineered with one or more exogenous coding sequences. These exogenous coding sequences may encode IL-10 and/or MMP9 and/or they may encode gene editing proteins such as CRISPR, nickases, and the like, that are capable of altering the cell's genome to increase the expression of endogenous IL-10 and/or endogenous MMP9. This could be achieved by editing the promoter or enhancer sequence, for example.

Known viral vectors for transfecting macrophages include lentivirus, adenoviruses and adeno-associated viruses (AAV).

As used herein, “coding” refers to the ability of sequences of nucleotides, such as a gene, a cDNA, or an mRNA, to serve as templates for synthesis of macromolecules in biological processes such as a defined sequence of amino acids. Thus a coding sequence may be any suitable nucleic acid sequence which provides the instructions to synthesise the relevant entity (e.g., IL-10 or MMP9). The coding sequences may be included in the same vector/construct or on different vectors/constructs.

As used herein, “exogenous” refers to any material, notably genetic material, introduced from or produced outside a particular cell. As used herein, in some embodiments, the exogenous coding sequence or engineered macrophage encode/express IL-10 or MMP9. In some embodiments, the IL-10 and/or MMP9 are human. It will be understood by those skilled in the art that variations to the sequence of these genes/coding sequences is also encompassed in this invention. Ideally, the genes/coding sequences are human. The genes/coding sequences may be codon optimised. The gene/coding sequences may be adjusted, and it will be appreciated if the reference sequence is RNA, then a different nucleotide may be present in a DNA vector.

Preferably, the sequence of the IL-10 coding sequence provided is at least 80% similar or homologous to NCBI Reference Sequence: NM_000572.3 (mRNA sequence of human IL10), preferably at least 85%, at least 90%, at least 95%, 96%, 97%, 98%, 99% similar to the natural sequence.

Preferably, the sequence of the MMP9 coding sequence provided is at least 80% similar or homologous to NCBI Reference Sequence: NM_004994.3 (Homo sapiens matrix metallopeptidase 9 (MMP9), mRNA), preferably at least 85%, at least 90%, at least 95%, 96%, 97%, 98%, 99% similar to the natural sequence.

Preferably, the IL-10 protein expressed is preferably at least 80% similar or homologous to the sequence presented as SEQ ID. No. 4, preferably at least 85%, at least 90%, at least 95%, 96%, 97%, 98%, 99% similar to SEQ ID No. 4.

Preferably, the MMP9 protein expressed is preferably at least 80% similar or homologous to the sequence presented as SEQ ID. No. 6, preferably at least 85%, at least 90%, at least 95%, 96%, 97%, 98%, 99% similar to SEQ ID No. 6.

In some embodiments, the macrophage is non-virally engineered, and is engineered with a nucleic acid vector. In some embodiments, the macrophage is transfected with a DNA vector e.g. a naked DNA vector. Suitably, the DNA vector may encode both IL-10 and MMP9 on a single vector In some embodiments, the nucleic acid vector is not derived from a viral genome. In some embodiments the macrophage is transfected with one or more free nucleic acids or vectors. In some embodiments, the macrophage is transfected with a RNA vector. In some embodiments, the macrophage is transfected with mRNA. Suitably, the macrophage is transfected with a single mRNA construct expressing IL-10 and MMP9.

In some embodiments, the IL-10 and MMP9 are provided to the macrophage as mRNA. Thus, the engineered cell is provided with exogenous mRNA molecules. The MMP9 and IL-10 may be on the same mRNA molecule, or on separate mRNA molecules. Preferably, the MMP9 and IL-10 are on the same mRNA molecule. Further preferably, the m RNA is a bi-cistronic vector that encodes both MMP9 and IL-10. Suitably, the mRNA molecules comprises a sequence as described in any one of SEQ ID No. 1 to 3, 10, 13 or 14.

In some embodiments, the mRNA comprises a sequence encoding IL-10. In some embodiments, the mRNA comprises a sequence at least 80% identical to SEQ ID NO: 13. In some embodiments, the mRNA comprises a sequence at least 85%, at least 90%, at least 95%, at least 96%, at least 97%, at least 98% or at least 99% identical to SEQ ID NO: 13. In a preferred embodiment, the mRNA comprises the sequence as described in SEQ ID NO: 13.

In some embodiments, the mRNA comprises a sequence encoding MMP9. In some embodiments, the mRNA comprises a sequence at least 80% identical to SEQ ID NO: 14. In some embodiments, the mRNA comprises a sequence at least 85%, at least 90%, at least at least 96%, at least 97%, at least 98% or at least 99% identical to SEQ ID NO: 14. In a preferred embodiment, the mRNA comprises the sequence as described in SEQ ID NO: 14.

In some embodiments, the macrophage is engineered with mRNA encoding both IL-10 and MMP9. In some embodiments, the macrophage is engineered with mRNA which comprises both the sequence as described in SEQ ID NO: 13 and the sequence as described in SEQ ID NO: 14. In some embodiments, the sequences as described in SEQ ID NOs 13 and 14 on the same mRNA molecule. In some embodiments, the sequences as described in SEQ ID NOs 13 and 14 are present on separate molecules. In a preferred embodiment, the mRNA comprises the sequences as described in SEQ ID NOs 13 and 14 on the same mRNA molecule, separated by mRNA encoding a self-cleaving linker. In preferred embodiments, the self-cleaving linker has an amino acid sequence as described in SEQ ID NO: 9. In preferred embodiments, the mRNA encoding the self-cleaving linker has a sequence as described in SEQ ID NO: 15.

In some embodiments, the mRNA construct expresses IL-10 fused to MMP9 via a cleavable linker. In some embodiments, the linker is p2A. p2A may have an amino acid sequence as described in SEQ ID No. 9, which may be encoded by an mRNA sequence as described in SEQ ID No. 8 or 15. In preferred embodiments, p2A is encoded by an mRNA sequence as described in SEQ ID NO: 15. p2A is part of a larger family of 2A self-cleaving peptides, or 2A peptides. This class of peptides are 18-22 amino acid-long peptides, which can induce ribosomal skipping during translation of a protein in a cell. These peptides share a core sequence motif of DxExNPGP. This permits the IL-10 and MMP9 to be expressed as separate proteins despite their provisions in the same mRNA molecule.

In preferred embodiments, both IL-10 and MMP9 are expressed from the mRNA molecule. In some embodiments, the mRNA molecule comprises a sequence at least 80% identical to SEQ ID NO: 10. In some embodiments, the mRNA molecule comprises a sequence at least 85%, at least 90%, at least 95%, at least 96%, at least 97%, at least 98% or at least 99% identical to SEQ ID NO: 10. In preferred embodiments, the mRNA comprises the sequence as described in SEQ ID NO: 10. In especially preferred embodiments, the mRNA comprises the sequence as described in SEQ ID NO: 10 further comprising a polyA tail, optionally between 65 and 250 residues long, preferably 90 to 120 residues long, preferably about 90 residues long, and/or a 5′ cap. In an especially preferred embodiment, the mRNA comprises the sequence as described in SEQ ID NO: 10 further comprising a polyA tail, optionally between 65 and 250 residues long, preferably 90 to 120 residues long, such as 90 residues long, and a 5′ cap. In a preferred embodiment, the exogenous mRNA sequence comprises a sequence at least 85%, at least 90%, at least 95%, at least 96%, at least 97%, at least 98% or at least 99% identical to the sequence set forth in SEQ ID NO: 16. In a particularly preferred embodiment, the exogenous mRNA sequence comprises the sequence set forth in SEQ ID NO: 16, preferably further comprising a 5′ cap.

In some embodiments, the exogenous mRNA sequence comprises a 5′ Untranslated Region sequence (5′UTR) at least 85%, at least 90%, at least 95%, at least 96%, at least 97%, at least 98% or at least 99% identical to the sequence set forth in SEQ ID NO: 19. In preferred embodiments, the exogenous mRNA sequence comprises a 5′ Untranslated Region sequence (5′UTR) at least 85%, at least 90%, at least 95%, at least 96%, at least 97%, at least 98% or at least 99% identical to the sequence set forth in SEQ ID NO: 17. In a preferred embodiment, the exogenous mRNA sequence comprises a 5′ UTR sequence as set forth in SEQ ID NO: 17.

In some embodiments, the exogenous mRNA sequence comprises a 3′ Untranslated Region sequence (3′UTR) at least 85%, at least 90%, at least 95%, at least 96%, at least 97%, at least 98% or at least 99% identical to the sequence set forth in SEQ ID NO: 20. In preferred embodiments, the exogenous mRNA sequence comprises a 3′ Untranslated Region sequence (3′UTR) at least 85%, at least 90%, at least 95%, at least 96%, at least 97%, at least 98% or at least 99% identical to the sequence set forth in SEQ ID NO: 18. In a preferred embodiment, the exogenous mRNA sequence comprises a 3′ UTR sequence as set forth in SEQ ID NO: 18. In a preferred embodiment, the exogenous mRNA comprises both a 5′UTR sequence as set forth in SEQ ID NO: 17, and a 3′ UTR sequence as set forth in SEQ ID NO: 18.

Alternatively or additionally, and IRES site may be inserted between the coding sequences, which the skilled person will appreciate will interrupt expression to form two separate proteins.

In some embodiments, the mRNA molecule(s) contain a poly A tail. A poly-A tail is a long chain of adenine nucleotides that is added to mRNA molecule during RNA processing to increase the stability of the molecule. A suitable poly-A tail is between 65 and 250 residues long, preferably 90 to 120 residue long. The poly-A tail makes the RNA molecule more stable and prevents its degradation.

In some embodiments, the mRNA molecules contains at least one cap. The 5′-cap is a hallmark of eukaryotic mRNA. Chemically, the 5′-cap consists of an inverted 7-methylguanosine connected to the rest of the eukaryotic mRNA via a 5′-5′ triphosphate bridge. This so-called “cap0” contributes to, inter alia, stabilization of eukaryotic mRNA, initiation of translation and mRNA decay. Artificial cap structures have been designed, to increase the success of in vitro translation. Synthetic anti-reverse cap analogues include extra methylation and trinucleotide CleanCap®AG, methylated at first adenosine. At least one cap may be a synthetic cap analogue, preferably a CleanCap®.

In some embodiments, the mRNA is modified RNA. In some embodiments, the mRNA modification comprises a chemical modification of uridine and/or a chemical modification of cytidine. In some embodiments, the mRNA modification comprises pseudouridine, N1-Methylpseudouridine, 5-methoxy-uridine, 5-methyl-cytidine, preferably 5-methoxy-uridine.

In an embodiment, the mRNA is modified with 5-methoxy-uridine and includes at least one CleanCap®.

In some embodiments, the mRNA construct comprises the sequence of SEQ ID NO. 2 or a sequence that is at least 85%, at least 90%, at least 95%, 96%, 97%, 98%, 99% similar or homologous to SEQ ID No. 2.

In some embodiments, the macrophage is engineered using a nucleic acid vector. In some embodiments the macrophage is transfected via electroporation. Other suitable methods of transfection include nucleofection.

In some embodiments, the mRNA is delivered to the macrophage via nanoparticles. In some embodiments, the nanoparticles are lipid nanoparticles, which may be as described in, for example, U.S. Pat. Nos. 8,058,069, 8,492,359, 8,822,668, 9,364,435, 9,504,651, and 11,141,378.

In some embodiments, the macrophage is autologous or allogenic to the subject. In some embodiments, the macrophage is derived from a progenitor cell, such as an iPSC, a hematopoietic stem cell, or a monocyte.

In some embodiments, the macrophage is derived from monocytes by culturing the latter under appropriate conditions. Such conditions may suitably include:

An in vitro method of producing macrophages comprising:
(b) Culturing monocytes in medium for 3-5 days to produce macrophages, wherein the medium comprises one or more growth factors to stimulate macrophage production;

Wherein step (a) takes place entirely in the same medium.

Suitably the medium is suitable for generating macrophages from monocytes. Suitably the medium is a T-cell medium. Suitably the medium may be selected from: X-Vivo 10, X-Vivo 15, TexMACS, AIMv, RPMI, DMEM, and DMEM/F12. Suitably the medium is TexMACS (Miltenyi).

Suitably the medium is serum-free. Suitably the medium is xenoprotein-free. Suitably the medium is GMP-compliant.

Suitably the medium may contain one or more factors. Suitable factors include growth factors, polysaccharides, cytokines and chemokines. Suitable factors may include: MCSF, GM-CSF. Suitably therefore the factors are growth factors. Suitably, the one or more factors are GMP-compliant. In one embodiment, the medium may comprise one or more growth factors may include MCSF or GM-CSF. Monocytes are most commonly cultured with either MCSF or GM-CSF. Culturing monocytes with GM-CSF skews them towards an “inflammatory” phenotype whereas culturing monocytes with MCSF skews them towards a “pro-restorative” phenotype. In other embodiments, the one or more growth factors does not include the combination of MCSF and GM-CSF. Thus, if M-CSF is used as the growth factor to generate macrophages in any of the methods of the invention, it may be preferred that GM-CSF is not also used to generate the macrophages from monocytes. This applies to the step of culturing monocytes until macrophages are generated.

According to a second aspect of the present invention, there is provided a population of engineered macrophages as described herein. Said population may be suitable for use in therapy as hereinbefore described.

According to a third aspect of the present invention, there is provided a composition comprising an engineered macrophage. In some embodiments, the engineered macrophage composition is for use in therapy as hereinbefore described or a population of engineered macrophages for use in therapy as hereinbefore described. The composition may also comprise other components that are pharmaceutically acceptable, such as suitable cell media, excipients and the like.

According to any aspect of the present invention, suitably the use comprises administering an effective amount of the engineered macrophage to the subject.

According to any aspect of the present invention, the use may comprise delivering the engineered macrophage to a subject by systemic administration, suitably by systemic injection. In some embodiments, administration is by local injection, e.g. for kidney and lung. In some embodiments, administration is by nebulizer into the lung. Peripheral vein injection is favoured for liver conditions in order to avoid invasive procedures in cirrhotic patients, for example. Local injection, such as renal artery for kidney conditions may be better tolerated.

It is preferred that the macrophage for use in therapy is engineered ex vivo and delivered to the patient.

However, in some embodiments, the macrophage may be engineered in vivo. In some embodiments, the macrophage is engineered in vivo via the administration to a subject of a preparation of exogenous coding sequence(s) for IL-10 and MMP9 suitable for transfecting a macrophage. The preparation may include any of the exogenous coding sequences discussed herein. Suitable delivery vehicles for in vivo engineering may include targeting molecules for macrophages by virtue of their cell surface markers. Should an in vivo transfection of macrophages be envisaged, localized application of the preparation may be more effective, such as localized injection of the preparation to liver or kidneys, or nebulization to the lungs. Such treatment may be prepared as nanoparticles to assist macrophage targeting.

According to a fourth aspect of the present invention, there is provided a method of improving the migration of monocytes to the site of inflammation comprising the use of an engineered macrophage according to the first aspect of the invention, a population of engineered macrophages according to the second aspect of the invention or a composition according to the third aspect of the invention.

In some embodiments, the method polarizes the host monocytes/macrophages to a pro-restorative phenotype. In some embodiments, the method polarizes unpolarized host macrophages to an anti-inflammatory and/or pro-restorative phenotype. In some embodiments, the method polarizes inflammatory host macrophages to an anti-inflammatory and/or pro-restorative phenotype.

In some embodiments, a pro-restorative phenotype may be described using one or more of the following markers: an increase CD206 and/or CD163, a decrease in inflammatory markers such as CD86 and/or MHC class II (HLA-DR). These increases/decreases are as compared to non-polarised (resting) or pro-inflammatory macrophages. In terms of secretion profile, these macrophages are expected not to express TNFa, IFNg and IL1b, normally associated with a pro-inflammatory and pro-fibrotic profile. In preferred embodiments, the engineered macrophage expresses CD206 at a 5-fold greater level than non-engineered, non-polarised cells, such as those described in WO2019/17559.

In some embodiments, the engineered macrophages do not secrete TNFa, IFNg, IL1b, IL-12p70 and/or IL-2. In preferred embodiments, the engineered macrophages secrete less than 40 pg/ml TNFa. In some embodiments, the engineered macrophages secrete the same or similar levels of TNFa, IFNg, IL1b, IL-12p70 and/or IL-2 as non-engineered, non-polarised cells.

The engineered macrophages secrete IL-10 and/or MMP9. In preferred embodiments, the engineered macrophage secretes at least 10,000 μg/ml IL-10. In preferred embodiments, the engineered macrophage secretes at least 200 ng/ml MMP9. In some embodiments, the engineered macrophages exhibit enhanced MMP activity. In preferred embodiments, the engineered macrophages exhibit MMP activity 1.5-fold greater than that of non-engineered, non-polarized cells. In some embodiments, the engineered macrophages secrete IL-6 and/or CXCL8.

According to another aspect of the present invention, there is provided a method of producing an engineered macrophage expressing IL-10 and MMP9. In some embodiments, the method comprises introducing nucleic acid comprising at least one sequence encoding IL-10 and at least one sequence encoding MMP9 into the macrophage or cell from which it is derived.

In a particular embodiment, the method comprises transiently transfecting a macrophage with an mRNA construct comprising at least one sequence encoding IL-10 and at least one sequence encoding MMP9. In another aspect, there is provided a method of producing an engineered macrophage expressing IL-10, such that the engineered macrophage secretes at least 10,000 pg/mL IL-10, comprising introducing a nucleic acid encoding IL-10 into the macrophage or cell from which it is derived. In some embodiments the method further comprises contacting the macrophage with an anti-inflammatory treatment after transfection, e.g. IL-4 and IL-13. In some embodiments the method comprises contacting the macrophage with IL-4 and IL-13. In some embodiments, the macrophages are incubated with IL-4, IL-13 and M-CSF.

In some embodiments, the method comprises electroporation. In some embodiments, the macrophage is contacted with an anti-inflammatory treatment after electroporation.

In certain embodiments, the invention provides:

1. An engineered macrophage which comprises an exogenous coding sequence for IL-10 and an exogenous coding sequence for MMP9.

2. An engineered macrophage of embodiment 1 wherein expression of said exogenous coding sequences has a synergistic effect in restoring MMP activity when compared to engineered macrophages comprising an exogenous sequence for IL-10 alone and/or a synergistic effect in monocyte recruitment by the macrophages.

3. The engineered macrophage of embodiment 1 or embodiment 2 wherein said macrophage and/or coding sequences are human.

4. The engineered macrophage of any one of embodiments 1 to 3 wherein said exogenous coding sequences are present on one or more nucleic acid molecules or are integrated into the genome of said macrophage.

5. The engineered macrophage of embodiment 4 wherein said nucleic acid molecule(s) are DNA or RNA molecules, preferably mRNA molecules.

6. The engineered macrophage of embodiment 5 wherein the mRNA molecules contain chemically modified residues, preferably modified uracil residues, and optionally at least one synthetic cap.

7. The engineered macrophage of any preceding embodiment wherein the exogenous coding sequence for IL-10 is on the same nucleic acid as the exogenous coding sequence for MMP9.

8. The engineered macrophage according to any preceding embodiment, wherein the macrophage is engineered to overexpress IL-10, preferably wherein the secreted IL-10 protein level is greater than about 300 pg/ml in a cell concentration of 4×10⁶/ml.

9. The engineered macrophage according to any preceding embodiment, wherein the level of metalloproteinase activity is at least 1.5 times the metalloproteinase activity of a non-engineered macrophage.

10. The engineered macrophage according to any one of embodiments 1 to 7 wherein the metalloproteinase activity is restored relative to the reduced metalloproteinase activity in a macrophage engineered with IL-10 coding sequence alone.

11. The engineered macrophage according to any preceding embodiment, wherein said macrophage is transiently transfected, optionally via electroporation.

12. The engineered macrophage of embodiment 10 wherein the transfection is non-viral.

13. The engineered macrophage of any preceding embodiment wherein said macrophage has a pro-restorative phenotype.

14. A population of engineered macrophages according to any preceding embodiment.

15. A therapeutic composition comprising a population of macrophages according to embodiment 14 plus a pharmaceutically acceptable medium.

16. An engineered macrophage of any one of embodiments 1 to 13, a population of macrophages of embodiment 14 or a composition of embodiment 15 for use in therapy.

17. An engineered macrophage, population or composition according to embodiment 16 wherein said therapy is administered to a subject in need thereof.

18. An engineered macrophage of any one of embodiments 1 to 13, a population of macrophages of embodiment 14 or a composition of embodiment 15 for use in treating an inflammatory condition in a subject.

19. An engineered macrophage, population or composition according to embodiment 17 or embodiment 18 wherein said macrophages are autologous or allogenic to the subject.

20. An engineered macrophage, population or composition according to embodiment 18 or embodiment 19 wherein the condition is a chronic inflammatory condition with a fibrotic element, optionally wherein the condition is organ damage associated with chronic inflammation.

21. An engineered macrophage, population or composition according to embodiment 18 or embodiment 19 wherein the condition is acute-on-chronic liver failure (ACLF).

22. A method of improving the migration of monocytes to an area of inflammation comprising the use of an engineered macrophage, population of engineered macrophages or a composition according to any of the preceding embodiments.

23. A method according to embodiment 22, wherein the method polarizes the host monocytes/macrophages to a pro-restorative phenotype and/or away from a pro-inflammatory phenotype.

24. A method of producing an engineered macrophage according to any one of embodiments 1 to 13, comprising transiently transfecting a macrophage with an mRNA molecule encoding IL-10 and/or MMP9.

25. A method according to embodiment 24, comprising and contacting the macrophage with IL-4 and IL-13 before, during or after the transfection.

26. A method according to embodiment 24 or 25, wherein the mRNA molecules encoding IL-10 and MMP9 are co-transfected using a bi-cistronic vector, linked by a p2A linker sequence.

27. An engineered macrophage according to any one of embodiments 1 to 13, wherein the macrophage is engineered with a mRNA construct encoding a human IL-10 fused to a human MMP9 protein via a cleavable linker.

28. A method of treating inflammation and/or fibrosis comprising administering to a subject in need thereof a therapeutically effective amount of engineered macrophages according to any one of embodiments 1-13.

The invention also provides the following embodiments, which may be combined with any other embodiments:

1. An engineered macrophage engineered to overexpress IL-10.

2. The engineered macrophage of embodiment 1, wherein the macrophage secretes IL-10 at a culture supernatant concentration of at least 10,000 pg/ml when cultured in vitro at a cell concentration of 4×10⁶/ml.

3. The engineered macrophage of embodiment 1 or embodiment 2, wherein the macrophage is additionally engineered to overexpress MMP9.

4. The engineered macrophage of embodiment 3, wherein the engineered macrophage comprises an exogenous coding sequence for IL-10 and an exogenous coding sequence for MMP9.

5. The engineered macrophage of 4 wherein expression of said exogenous coding sequences has a synergistic effect in restoring MMP activity when compared to engineered macrophages comprising an exogenous sequence for IL-10 alone and/or a synergistic effect in monocyte recruitment by the macrophages.

6. The engineered macrophage of any one of embodiments 1-5, wherein said macrophage and/or coding sequences are human.

7. The engineered macrophage of any one of embodiments 4-6, wherein the exogenous coding sequence for IL-10 encodes a protein with an amino acid sequence at least 85%, at least 90%, at least 95%, 96%, 97%, 98% or 99% identical to SEQ ID NO: 4, optionally wherein the IL-10 protein comprises an amino acid sequence identical to SEQ ID NO: 4.

8. The engineered macrophage of any one of embodiments 4-7, wherein the exogenous coding sequence for MMP9 encodes a protein with an amino acid sequence at least 85%, at least 90%, at least 95%, 96%, 97%, 98% or 99% identical to SEQ ID NO: 6, optionally wherein the IL-10 protein comprises an amino acid sequence identical to SEQ ID NO: 6.

9. The engineered macrophage of any one of embodiments 4, 5, 7 or 8 wherein said exogenous coding sequences are present on one or more nucleic acid molecules or are integrated into the genome of said macrophage.

10. The engineered macrophage of embodiment 9 wherein said nucleic acid molecule(s) are DNA or RNA molecules, preferably mRNA molecules, optionally wherein the IL-10 and MMP9 are expressed from the same mRNA molecule, further optionally wherein the mRNA molecule encodes IL-10 and MMP9 linked by a linker sequence, further optionally wherein the linker is a self-cleaving 2A linker, further optionally wherein the linker is p2A.

11. The engineered macrophage of embodiment 10, wherein the nucleic acid molecule(s) are mRNA molecule(s), comprising a sequence at least 85%, at least 90%, at least 95%, at least 96%, at least 97%, at least 98% or at least 99% identical to SEQ ID NO: 13, optionally wherein the nucleic acid comprises SEQ ID NO: 13.

12. The engineered macrophage of embodiments 10 or 11, wherein the nucleic acid molecule(s) are mRNA molecule(s), comprising a sequence at least 85%, at least 90%, at least 95%, at least 96%, at least 97%, at least 98% or at least 99% identical to SEQ ID NO: 14, optionally wherein the nucleic acid comprises SEQ ID NO: 14.

13. The engineered macrophage of embodiments 10-12, wherein the nucleic acid molecule is an mRNA molecule which encodes IL-10 and MMP9 linked by a linker sequence, and wherein the linker sequence encodes a protein comprising an amino acid sequence as described in SEQ ID NO: 7, optionally wherein the protein encoded by the linker sequence comprises an amino acid sequence as described in SEQ ID NO: 9.

14. The engineered macrophage of embodiments 10-13, wherein the nucleic acid molecule is an mRNA molecule which encodes IL-10 and MMP9 linked by a linker sequence, and wherein the linker sequence comprises mRNA with a sequence as described in SEQ ID NO: 15.

15. The engineered macrophage of embodiments 10-14, wherein the nucleic acid molecule is an mRNA molecule comprising a sequence at least 80% identical to SEQ ID NO: 10, preferably at least 85%, at least 90%, at least 95%, at least 96%, at least 97%, at least 98%, or at least 99% identical to SEQ ID NO: 10, optionally wherein the mRNA further comprises a polyA tail between 65 and 250 residues long, preferably 90 to 120 residues long, preferably about, and/or a 5′ cap.

16. The engineered macrophage of embodiments 10-15, wherein the nucleic acid molecule is an mRNA molecule comprising a sequence at least 80% identical to SEQ ID NO: 16, preferably at least 85%, at least 90%, at least 95%, at least 96%, at least 97%, at least 98%, or at least 99% identical to SEQ ID NO: 16, optionally wherein the mRNA further comprises a 5′ cap.

17. The engineered macrophage of embodiment 16 wherein the mRNA molecules contain chemically modified residues, preferably modified uracil residues, and optionally at least one synthetic cap.

18. The engineered macrophage of any of embodiments 4 to 17, wherein the exogenous coding sequence for IL-10 is on the same nucleic acid as the exogenous coding sequence for MMP9.

19. The engineered macrophage according to any of the preceding embodiments, wherein the macrophage is engineered by editing the endogenous promoters of the IL-10 gene and/or the MMP9 gene, or wherein the macrophage is engineered by modulating the expression of an endogenous silencing RNA, or introducing an exogenous silencing RNA sequence, optionally wherein the silencing RNA is miRNA.

20. The engineered macrophage according to any preceding embodiment, wherein the level of metalloproteinase activity is at least 1.5 times the metalloproteinase activity of a non-engineered macrophage.

21. The engineered macrophage of any one of the preceding embodiments wherein the engineered macrophage has an at least two-fold reduced expression of CD86 compared to non-engineered, non-polarised cells.

22. The engineered macrophage of any one of the preceding embodiments wherein the engineered macrophage has an at least two-fold reduced expression of HLA-DR compared to non-engineered, non-polarised cells.

23. The engineered macrophage of any one of the preceding embodiments wherein the engineered macrophage has an at least 1000-fold increased secretion of IL-10 compared to non-engineered, non-polarised cells.

24. The engineered macrophage of any one of the preceding embodiments wherein the engineered macrophage has an at least 10-fold increased secretion of MMP3 compared to non-engineered, non-polarised cells.

25. The engineered macrophage of any one of the preceding embodiments wherein the engineered macrophage has an at least 20-fold increased secretion of MMP10 compared to non-engineered, non-polarised cells.

26. The engineered macrophage of any one of the preceding embodiments wherein the macrophage secretes IL-10 at a culture supernatant concentration of at least 10,000 pg/ml when cultured in vitro at a cell concentration of 4×10⁶/ml.

27. The engineered macrophage of any one of the preceding embodiments wherein the macrophage secretes MMP9 at a culture supernatant concentration of at least 200 ng/ml when cultured in vitro at a cell concentration of 4×10⁶/ml.

28. The engineered macrophage of any one of the preceding embodiments wherein the engineered macrophage has an at least 5-fold increased expression of CD206 compared to monocytes.

29. The engineered macrophage of any one of the preceding embodiments wherein the engineered macrophage has an at least 5-fold increased expression of 25F9 compared to monocytes.

30. The engineered macrophage of any one of the preceding embodiments wherein the engineered macrophage has an at least ten percent reduced expression of CD80 compared to non-engineered, non-polarised cells.

31. The engineered macrophage of any one of the preceding embodiments wherein the macrophage secretes TNF-α at a culture supernatant concentration of up to 40 pg/ml when cultured in vitro at a cell concentration of 4×10⁶/ml.

32. The engineered macrophage of any one of the preceding embodiments, wherein the engineered macrophage has phagocytic ability at least equivalent to a non-engineered, non-polarised cell.

33. The engineered macrophage according to any one of embodiments 1-32 wherein the metalloproteinase activity is restored relative to the reduced metalloproteinase activity in a macrophage engineered with IL-10 coding sequence alone.

34. The engineered macrophage according to any preceding embodiment, wherein said macrophage is transiently transfected, optionally via electroporation.

35. The engineered macrophage of embodiment 34, wherein the transfection is non-viral.

36. The engineered macrophage of any preceding embodiment wherein said macrophage has a pro-restorative phenotype.

37. A population of engineered macrophages according to any preceding embodiment.

38. A therapeutic composition comprising a population of macrophages according to embodiment 37 plus a pharmaceutically acceptable medium.

39. An engineered macrophage of any one of embodiments 1 to 36, a population of macrophages of embodiment 37, or a composition of embodiment 38, for use in therapy.

40. An engineered macrophage, population or composition according to embodiment 39 wherein said therapy is administered to a subject in need thereof.

41. An engineered macrophage of any one of embodiments 1 to 36, a population of macrophages of embodiment 37, or a composition of embodiment 38, for use in treating an inflammatory condition in a subject.

42. An engineered macrophage, population or composition according to embodiments 40 or 41, wherein said macrophages are autologous or allogenic to the subject.

43. An engineered macrophage, population or composition according to embodiment 41, wherein the inflammatory condition is a liver injury, optionally chronic liver injury.

44. An engineered macrophage, population or composition according to embodiment 41 or 43, wherein the condition is a chronic inflammatory condition with a fibrotic element, optionally wherein the condition is organ damage associated with chronic inflammation.

45. The engineered macrophage, population or composition according to any one of embodiments 41, 43 or 44, wherein the condition is fibrosis, and wherein fibrosis is in or affects an organ selected from the group consisting of: liver, lung, heart, kidney, pancreas, skin, gastrointestinal, bone marrow, hematopoietic tissue, nervous system, eye and a combination thereof.

46. The engineered macrophage, population or composition of any of embodiments 43 or 44, wherein the condition is liver cirrhosis.

47. The engineered macrophage, population or composition of embodiment 46, wherein the liver cirrhosis resulted from at least one disease or condition selected from the group consisting of: non-alcoholic fatty liver disease (NAFL) (e.g., non-alcoholic fatty liver disease (NAFLD) or non-alcoholic steatohepatitis (NASH)), alcoholic liver disease (e.g., alcoholic fatty liver disease (AFLD) or alcoholic steatohepatitis (ASH)), mechanical trauma to the liver, biliary obstruction, autoimmune hepatitis, iron overload, Hepatitis B infection (HBV) and Hepatitis C infection (HCV).

48. An engineered macrophage population or composition of embodiment 46, wherein the liver cirrhosis resulted from steatotic liver disease (SLD), optionally wherein the steatotic liver disease is metabolic dysfunction-associated steatotic liver disease, metabolic-associated steatohepatitis, Met-ALD or Cryptogenic SLD.

49. The engineered macrophage, population or composition of embodiments 46-48, wherein the liver cirrhosis is selected from compensated cirrhosis and decompensated cirrhosis.

50. An engineered macrophage, population or composition according to any of embodiments 41-49 wherein the condition is acute-on-chronic liver failure (ACLF).

51. An engineered macrophage, population or composition according to embodiments 46-49, for use in treating a subject that has recovered from their first hepatic decompensation event (recompensated), optionally wherein the first hepatic decompensation event required the subject's hospitalization, preferably wherein the subject has not undergone an additional hepatic decompensation event after having recovered from the first decompensation event.

52. The engineered macrophage, population or composition according to embodiment 46-49 and 51, wherein the subject exhibits or has recovered from one or more clinical signs of hepatic decompensation selected from the list consisting of jaundice, ascites, hepatic encephalopathy, hepatorenal syndrome, variceal haemorrhage and gastrointestinal haemorrhage.

53. The engineered macrophage, population or composition according to any of embodiments 1-52 wherein the macrophages are derived from human monocyte-derived macrophages (hMDMs) or stem cells, optionally wherein the stem cells are induced pluripotent stem cells (iPSCs).

54. The engineered macrophage, population or composition according to embodiment 53, wherein the macrophages are derived from iPSCs, and the iPSCs are essentially devoid of functional HLA I and II complexes on their surface.

55. A method of improving the migration of monocytes to an area of inflammation comprising the use of an engineered macrophage, population of engineered macrophages or a composition according to any of the preceding embodiments.

56. A method according to embodiment 55, wherein the method polarizes the host monocytes/macrophages to a pro-restorative phenotype and/or away from a pro-inflammatory phenotype.

57. A method of producing an engineered macrophage according to any one of embodiments 1 to 36, comprising transiently transfecting a macrophage with an mRNA molecule encoding IL-10 and/or MMP9.

58. A method according to embodiments 57, comprising contacting the macrophage with IL-4, IL-13 and M-CSF before, during or after the transfection.

59. A method according to embodiments 57 or 58, wherein the mRNA molecules encoding IL-10 and MMP9 are co-transfected using a bi-cistronic vector, linked by a p2A linker sequence.

60. An engineered macrophage according to any one of embodiments 1 to 36, wherein the macrophage is engineered with a mRNA construct encoding a human IL-10 fused to a human MMP9 protein via a cleavable linker.

61. A method of treating inflammation and/or fibrosis comprising administering to a subject in need thereof a therapeutically effective amount of engineered macrophages according to any one of embodiments 1-36.

62. A method of polarising macrophages to a pro-restorative phenotype, wherein the polarised macrophages have an increased expression of CD163 and CD206, and a reduced expression of HLA DR and CD86 compared to cells not polarised to a pro-restorative phenotype, wherein the method comprises engineering the macrophage to express IL-10 and MMP9 above endogenous levels.

63. The method of embodiment 62, wherein the macrophage is engineered to express IL-10 and MMP9 by introducing exogenous nucleic acid comprising nucleotide sequences encoding IL-10 and MMP9.

64. The method of embodiment 63, wherein the nucleotide sequences encoding IL-10 and MMP9 are present on the same nucleic acid molecule.

65. The method of embodiment 63, wherein the nucleotide sequences encoding IL-10 and MMP9 are present on separate nucleic acid molecules.

66. The method of any one of embodiments 63-65, wherein the nucleic acid is mRNA.

67. A method of polarising macrophages to a pro-restorative phenotype, wherein the polarised macrophages have an increased expression of CD163 and CD206, and a reduced expression of HLA DR and CD86 compared to cells not polarised to a pro-restorative phenotype, wherein the method comprises engineering the macrophage to overexpress IL-10, optionally wherein the macrophage secretes IL-10 at a culture supernatant concentration of at least 10,000 pg/ml when cultured in vitro at a cell concentration of 4×10⁶/ml.

68. The method of embodiment 67, wherein the macrophage is engineered to express IL-10 by introducing exogenous nucleic acid comprising a nucleotide sequence encoding IL-10

69. The method of embodiment 67 or 68, wherein the nucleic acid is mRNA.

70. A method of improving cryoresilience in macrophages, comprising incubating the macrophages in medium comprising IL-4, IL-13 and M-CSF.

71. A method of cryopreserving macrophages, comprising incubating the macrophages in medium comprising IL-4, IL-13 and M-CSF prior to cryopreservation.

72. The method of embodiment 70 or 71, wherein the concentration of IL-4 and IL-13 in the medium are 20 ng/ml, the concentration of M-CSF is 100 ng/ml, and the macrophages are at a concentration of 4×10′ cells/ml.

73. The method of embodiments 70-72, wherein the cells are incubated overnight in the medium comprising IL-4, IL-13 and M-CSF.

74. Cryopreserved macrophages obtained by a method of any of embodiments 70-73.

According to a preferred embodiment, the invention provides an engineered macrophage engineered to overexpress IL-10, for use in a method of treating liver cirrhosis in a subject that has been hospitalised following their first hepatic decompensation event. In a preferred embodiment, the invention provides an engineered macrophage engineered to overexpress IL-10, for use in a method of treating liver cirrhosis in a subject that has been hospitalised following their first hepatic decompensation event, wherein the subject is treated with the engineered macrophage once the subject has recovered from their first hepatic decompensation event and prior to undergoing additional hepatic decompensation events. In a particularly preferred embodiment, the invention provides an engineered macrophage engineered to overexpress IL-10 and MMP9, for use in a method of treating liver cirrhosis in a subject that has been hospitalised following their first hepatic decompensation event, wherein the subject is treated with the engineered macrophage once the subject has recovered from their first hepatic decompensation event and prior to undergoing additional hepatic decompensation events.

According to a further preferred embodiment, the invention provides an engineered macrophage that is derived from an iPSC and that is engineered to overexpress IL-10. Preferably, the engineered iPSC-derived macrophage is for use in treating liver cirrhosis, in particular in a subject that has been hospitalised following their first hepatic decompensation event. In a particularly preferred embodiment, the engineered iPSC-derived macrophage is for use in treating liver cirrhosis, in particular in a subject that has been hospitalised following their first hepatic decompensation event, wherein the subject is treated with the engineered macrophage once the subject has recovered from their first hepatic decompensation event and prior to undergoing additional hepatic decompensation events.

According to a further preferred embodiment, the invention provides an engineered macrophage engineered to overexpress IL-10 and MMP9, wherein the macrophage comprises an exogenous mRNA encoding for IL-10 and MMP9, optionally separated by a cleavable linker. Preferably, the exogenous mRNA comprises the sequence set forth in SEQ ID NO: 10, a polyA tail, optionally between 65 and 250 residues long, preferably 90 to 120 residue longs and a 5′ cap. In another preferred embodiment, the exogenous mRNA sequence comprises the sequence set forth in SEQ ID NO: 16 and a 5′ cap. Preferably, the engineered macrophage is for use in treating liver cirrhosis, in particular in a subject that has been hospitalised following their first hepatic decompensation event. In a particularly preferred embodiment, the engineered macrophage is for use in treating liver cirrhosis, in particular in a subject that has been hospitalised following their first hepatic decompensation event, wherein the subject is treated with the engineered macrophage once the subject has recovered from their first hepatic decompensation event and prior to undergoing additional hepatic decompensation events.

According to a further preferred embodiment, the invention provides an engineered macrophage engineered that is derived from an iPSC and that is engineered to overexpress IL-10 and MMP9, wherein the macrophage comprises an exogenous mRNA encoding for IL-10 and MMP9, optionally separated by a cleavable linker. Preferably, the exogenous mRNA comprises the sequence set forth in SEQ ID NO: 10, a polyA tail, optionally between 65 and 250 residues long, preferably 90 to 120 residues long, preferably about 90 residues long, and/or a 5′ cap. In another preferred embodiment, the exogenous mRNA sequence comprises the sequence set forth in SEQ ID NO: 16 and a 5′ cap. Preferably, the iPSC-derived engineered macrophage is for use in treating liver cirrhosis, in particular in a subject that has been hospitalised following their first hepatic decompensation event. In a particularly preferred embodiment, the iPSC-derived engineered macrophage is for use in treating liver cirrhosis, in particular in a subject that has been hospitalised following their first hepatic decompensation event, wherein the subject is treated with the engineered macrophage once the subject has recovered from their first hepatic decompensation event and prior to undergoing additional hepatic decompensation events.

According to a further preferred embodiment, the invention provides an engineered macrophage engineered to overexpress IL-10, wherein the macrophage secretes IL-10 at a culture supernatant concentration of at least 10,000 pg/ml when cultured in vitro at a cell concentration of 4×10⁶/ml, for use in a method of treating liver cirrhosis, in particular in a subject that has been hospitalised following their first hepatic decompensation event. In a particularly preferred embodiment, the invention provides an engineered macrophage engineered to overexpress IL-10, wherein the macrophage secretes IL-10 at a culture supernatant concentration of at least 10,000 pg/ml when cultured in vitro at a cell concentration of 4×10⁶/ml, for use in a method of treating liver cirrhosis, in particular in a subject that has been hospitalised following their first hepatic decompensation event, wherein the subject is treated with the engineered macrophage once the subject has recovered from their first hepatic decompensation event and prior to undergoing additional hepatic decompensation events.

According to a further preferred embodiment, the invention provides an engineered macrophage engineered to overexpress IL-10 and MMP9, wherein the engineered macrophage: has a level of metalloproteinase activity is at least 1.5 times the metalloproteinase activity of a non-engineered macrophage; has an at least two-fold reduced expression of CD86 compared to non-engineered, non-polarised cells; an at least two-fold reduced expression of HLA-DR compared to non-engineered, non-polarised cells; has an at least 1000-fold increased secretion of IL-10 compared to non-engineered, non-polarised cells; has an at least 10-fold increased secretion of MMP3 compared to non-engineered, non-polarised cells; has an at least 20-fold increased secretion of MMP10 compared to non-engineered, non-polarised cells; has an at least 5-fold increased expression of CD206 compared to monocytes; has an at least 5-fold increased expression of 25F9 compared to monocytes; has an at least ten-percent reduced expression of CD80 compared to non-engineered, non-polarised cells; secretes IL-10 at a culture supernatant concentration of at least 10,000 pg/ml when cultured in vitro at a cell concentration of 4×10⁶/ml; secretes MMP9 at a culture supernatant concentration of at least 200 ng/ml when cultured in vitro at a cell concentration of 4×10⁶/ml; and secretes TNF-α at a culture supernatant concentration up to 40 pg/ml when cultured in vitro at a cell concentration of 4×10⁶/ml. In an especially preferred embodiment, the engineered macrophage is derived from an iPSC and the macrophage comprises an exogenous mRNA encoding for IL-10 and MMP9, optionally separated by a cleavable linker. Preferably, the iPSC-derived engineered macrophage is for use in treating liver cirrhosis, in particular in a subject that has been hospitalised following their first hepatic decompensation event. The present invention will now be described further with reference to the following headed sections. Any features under any of the sections may be combined with any of the aspects or embodiments of the invention in any workable order.

### DESCRIPTION

The following definitions are provided.
‘payload’ as used herein is a gene or genes of therapeutic interest, which is introduced via transfection to test its effect on macrophages.
‘non-polarized macrophage’ as used herein refers to a mature macrophage which has not received any further stimulation to induce particular functional capacity, non-polarized macrophages may also refer to naïve or non-activated macrophages.
‘mature macrophage’ refers to a macrophage which expresses mature cell surface markers, preferably CCR2−, CD14+ and 25F9+.

Macrophages may acquire various states, referred to as “polarisation”, which are usually, but simplistically, divided into two main extremes, “pro-inflammatory” (or classically-activated, “M1”, “M1-like”) and “pro-regenerative” (or “pre-restorative”, alternatively-activated, “M2”, “M2-like”, anti-inflammatory or anti-fibrotic). However, macrophages may adopt a state between these extremes which may be “unpolarised”, resting or naïve (M0) or point more towards an anti or pro-inflammatory state.

It is generally postulated that M1 macrophages are pro-inflammatory, whereas M2 macrophages are responsible for immunomodulation and wound-healing responses. However, it is increasingly clear that this binary classification does not address the more complex heterogeneity in vivo, where macrophages adopt distinct phenotypes and even switch between phenotypes in response to the myriad of stimuli to which they are exposed. These in vivo macrophage phenotypes are impossible to recapitulate exactly in tissue culture models, emphasizing the importance of the characterization of macrophages on the basis of function. Macrophages acquire a “pro-regenerative” state under the action of various factors in combination, including macrophage colony-stimulating factor (M-CSF), IL-4, IL-13, IL-10 and TGF-β. These macrophages mediate wound healing and tissue regeneration primarily.

Due to the complexity of macrophage biology, the categorisation to “M1” and “M2” can be considered over simplified. For example, “M2” macrophages are through to in fact be a spectrum depending on their environment. Further sub-categorisation of the pro-regenerative M2-like state has therefore been attempted, such as the following sub-categorisation suggested by Gharavi, A. T et al., “The role of macrophage subtypes and exosomes in immunomodulation”, Cell Mol Biol Lett 27, 83 (2022). In this sub-categorisation, for example:
“M2a” cells are thought to be anti-inflammatory, profibrotic and to have roles in allergy and wound healing. Such cells are categorised by the expression of IL-10, Il-1R, IL-27a, CCL1, CCL17, CCL18, CCL22, CD11b, CD45, CD206, YM1, RELMa, IGF1. DCIR, Stabilin 1, Factor XIII-A, Ly6C, TREM-2 and DC-SIGN. The M2a state may be acquired under the action of factors such as IL-4, IL-13, IL-10 and PPARg.
“M2b” cells are thought to be involved in activation of a T-helper 2 (Th2) type response, immune regulation and promoting tumour progression. Such cells are categorised by the expression of IL-6, TNF-α, CD86 and SPHK1. The M2b state may be acquired under the action of IL-1b or exposure to LPS.
“M2c” cells are associated with immunosuppression, phagocytosis, tissue repair and extracellular matrix remodelling. Such cells are categorised by the expression of IL-10, CXCL13, CD163, CD206, CXCR4, TGF-b and MerTK. The M2c state may be acquired under the action of IL-10, glucocorticoids, IL-6, IL-10, TNF-α and TLR stimulation.
“M2d” cells are associated with tumour progression, angiogenesis, and clearance of apoptotic tissue. Such cells are categorised by the expression of IL-10, VEGF and TGF-b. The M2d state may be acquired upon exposure to LPS.

Of the markers associated with each of these pro-restorative states, the engineered macrophages of the present invention may typically express IL-10, CCL22, CD11b, CD45, CD206, CD86, CD163 and CXCR4. The engineered macrophages of the present invention may also typically express MHC II, which is more usually associated with pro-inflammatory macrophages. Accordingly, it can be seen that the engineered macrophages of the present invention are distinct from the pro-restorative, anti-inflammatory macrophages described in the literature, as they express markers associated with multiple subtypes, for example, both M2a and M2c-like cells. However, in all aspects of the present invention, the engineered macrophage of the invention may express markers consistent with functional human monocyte derived macrophages, such as CD45, CD14, CD206, CCR2, CD163, CD169 and 25F9.

The engineered macrophages of the present invention (comprising IL-10 and MMP9) function as “Pro-restorative” macrophages. As described in e.g. Ramachandran et al, Proc Natl Acad Sci USA. 2012 Nov. 13; 109(46): E3186-E3195, pro-restorative macrophages have a loss of pro-inflammatory gene expression, increased expression of matrix-degrading enzymes, and enrichment of phagocytosis-related genes. Furthermore, the phenotype of pro-restorative macrophages falls outside the M1/M2 paradigm, highlighting the limitations of this classification in an in vivo setting. Pro-restorative macrophages play an important role in tissue remodelling e.g. fibrosis resolution.

The macrophages reported in Ramachandran et al have high MMP activity. Further, in FIG. 5 the authors show several genes regulated in the pro-restorative macrophages. Of note, they see an upregulation of Mrc1 (now CD206) which is also expressed in the macrophages of the Examples presented here. Further, they show low levels of TNFα and IL1b, as also demonstrated in the Examples presented here. Such may therefore be indicative of a pro-restorative phenotype. However, these macrophages described in this paper naturally express low levels of IL-10.

‘GMP-compliant’ as used herein means that the method complies with Good Manufacturing Practice principles and may be used interchangeably with ‘GMP-compatible’ and ‘GMP-graded’. By way of example a GMP-compliant medium has to be serum-free, antibiotic-free, animal substance free and xenoprotein-free. The WHO provides guidance on what is required for good manufacturing practice: “Chapter 1: WHO good manufacturing practices: Main principles for pharmaceutical products”. Quality Assurance of Pharmaceuticals: A compendium of guidelines and related materials—Good manufacturing practices and inspection. 2 (2nd updated ed.). WHO Press. pp. 17-18. ISBN 9789241547086.

As used herein, “UT”, “NTRx” or “UT N/T” refers to untransfected macrophages that are differentiated from monocytes in the same process used to differentiate the transfected macrophages, but without any further incubation with additional factors and/or transfection. Such macrophages are similar to the non-polarised, non-transfected cells used in the MATCH study described in WO2019175595 (so UT may also be described in the application as MATCH-like cells), however the macrophages in the MATCH study were matured for 7 days while the UT macrophages in the present application have been matured for 5 days.

As used herein, “UT+TR”, “NTRx+Tr” or “UT T” refers to untransfected cells (as described above) which have been treated similarly to the transfected cells (i.e. incubated with IL-4+IL-13+M-CSF following mock transfection). Incubation with IL-4+IL-13+M-CSF is described in more detail in the Examples herein.

Also described herein are hMDMs which are transfected with constructs encoding a specified product, such as MMP9 and/or IL-10, which have not been treated with IL-4+IL-13+M-CSF as described herein. Such cells are referred to as “TRx”, for example a cell transfected with IL-10 but without incubation with IL-4+IL-13+M-CSF, is denoted “IL-10 TRx”.

Also described herein are hMDMs which are transfected with constructs encoding a specified product, and have also been further treated with IL-4+IL-13+M-CSF. Such cells are denoted “TRx+TR”, such as “IL-10 TRx+TR”. Also described herein are hMDMs which are transfected with an exogenous bicistronic mRNA construct expressing IL-10 and MMP9. Such cells are denoted “RTX001”. Without wishing to be bound by theory or mechanism, overexpression of IL-10 and MMP9 mean that RTX001 cells may self-polarise to an anti-inflammatory and/or pro-restorative phenotype.

It is to be noted that the term “a” or “an” entity refers to one or more of that entity.

‘about’ means+/−10% of the value given, +/−9%, +/−8%, +/−7%, +/−6%, +/−5%, +/−4%, +/−3%, +/−2%, +/−1%, unless otherwise stated.

### Engineered Macrophages for Use as Cell Therapy

In a preferred embodiment, the invention relates to an engineered macrophage, wherein the macrophage comprises one or more exogenous coding sequence(s) for IL-10 and MMP9.

The invention relates to an engineered macrophage, wherein the macrophage is engineered to express IL-10 and MMP9. The macrophages may be engineered to express these proteins by inclusion of exogenous coding sequences. Said exogenous coding sequences may be extrachromosomal or integrated into the cell's genome. An engineered macrophage expresses the exogenous coding sequence(s) for IL-10 and MMP9.

In some embodiments, the engineered macrophages carry one or more exogenous sequence that can turn on endogenous expression of IL-10 and/or MMP9. Any macrophage that has been genetically modified by any means through an exogenous sequence (i.e., a sequence that is not a part of the natural macrophage genome) is an engineered macrophage according to the invention.

The engineered macrophages may have use in therapy, e.g. for use in treating an inflammatory condition in a subject, and/or a fibrotic condition.

Preferably, administration of the engineered macrophage to a subject is not associated with an inflammatory response. In particular, administration of the engineered macrophage to a subject is preferably not associated with an increased concentration of inflammatory cytokines in the plasma, such as IL-1b and/or TNF-a.

A ‘macrophage’ as used herein refers to a phagocytic cell which is responsible for detecting, engulfing and destroying pathogens and apoptotic cells, and which is produced through the differentiation of monocytes. An ‘engineered macrophage’ of the present invention is a macrophage that has been engineered to express IL-10 and/or MMP9. In particular, the expression is above endogenous levels, such that the engineered macrophages express IL-10 and/or MMP9 at greater levels than non-engineered cells. The macrophages may be engineered to express these proteins by inclusion of exogenous coding sequences. In some embodiments, the macrophage secretes IL-10 at a culture supernatant concentration of at least 10,000 pg/ml when cultured in vitro at a cell concentration of 4×10⁶/m. In some embodiments, the macrophage secretes MMP9 at a culture supernatant concentration of at least 200 ng/ml when cultured in vitro at a cell concentration of 4×10⁶/ml.

In some embodiments, the IL-10 amino acid sequence is encoded by an mRNA comprising the sequence of SEQ ID NO. 1. The natural mRNA sequence for IL-10 mRNA is provided at: NCBI Reference Sequence: NM_000572.3. Variants and homologues of this sequence are also included.

MMP9 (Matrix metallopeptidase 9) is a matrix metalloprotease, a type IV collagenase. MMP9 is also known as 92 kDa type IV collagenase, 92 kDa gelatinase or gelatinase B (GELB). Matrix metalloproteases (MMPs), also known as matrix metalloproteinases or matrixins are a family of peptidases, collectively capable of cleaving all components of the extracellular matrix (ECM). MMPs are also able to process bioactive mediators, such as growth factors, cytokines, chemokines, and cell-surface receptors. 25 mammalian MMPs have been identified, with varying roles in the maintenance of the ECM and processes of tissue repair, and both inhibitory and stimulatory roles in fibrosis.

In some embodiments, the MMP9 coding sequence is human MMP9 as described in NCBI Reference Sequence: NM_004994.3. In some embodiments, the amino acid sequence is encoded by an mRNA comprising the sequence of SEQ ID NO. 3, or a variant that is at least 85%, at least 90%, at least 95%, 96%, 97%, 98%, 99% similar to SEQ ID No. 3.

In some embodiments, the mRNA is modified. In some embodiments, the mRNA modification is selected from a chemical modification of uridine and/or a chemical modification of cytidine. pseudouridine. In some embodiments, the mRNA modification comprises pseudouridine, N1-Methylpseudouridine, 5-methoxy-uridine, 5-methyl-cytidine, preferably 5-methoxy-uridine. In preferred embodiments, all endogenous uridines have been substituted with 5-methoxy-uridine.

In some embodiments, the mRNA comprises a poly-A tail at the 3′ end. A suitable poly-A tail is between 65 and 250 residues long, preferably 90 to 120 residue long. In preferred embodiments, the 3′ and 5′ UTRs are modified with respect to the endogenous 3′ and 5′ UTRs. In some embodiments, the polyA tail is 120 residues long. In preferred embodiments, the polyA tail is longer than the endogenous polyA tail. In an especially preferred embodiment, the polyA tail is 90 residues long. In an especially preferred embodiment, the mRNA comprises the sequence of SEQ ID No. 10, or a variant which is at least 85%, at least 90%, at least 95%, at least 96%, at least 97%, at least 98%, at least 99% similar to SEQ ID NO. 10. In another preferred embodiment, the exogenous mRNA sequence comprises a sequence at least 85%, at least 90%, at least 95%, at least 96%, at least 97%, at least 98% or at least 99% identical to the sequence set forth in SEQ ID NO: 16. In a particularly preferred embodiment, the exogenous mRNA sequence comprises the sequence set forth in SEQ ID NO: 16. As described herein, the endogenous uridines in SEQ ID NOs 10 and 16 are substituted with 5-methoxy-uridine. In a preferred embodiment, the mRNA comprises the sequence of SEQ ID NO: 10, a polyA tail 90 residues long and a 5′ cap. In a preferred embodiment, the mRNA comprises the sequence of SEQ ID NO: 16 and a 5′ cap.

In some embodiments, the mRNA is resistant to degradation. In some embodiments, the mRNA is not immunogenic.

In some embodiments, the IL-10 and the MMP9 proteins are expressed by separate exogenous coding sequence or nucleic acid molecules. In some embodiments, the IL-10 and MMP9 proteins are expressed as fusion proteins encoded by a single mRNA molecule, linked by a cleavable linker.

In some embodiments, the mRNA comprises the sequence of SEQ ID NO. 2 or a variant which is at least 85%, at least 90%, at least 95%, 96%, 97%, 98%, 99% similar to SEQ ID No. 2.

In some embodiments, the mRNA has been modified in any one or both of the 5′ and 3′ ends by any means that enhances the stability and/or ability to express the encoded protein(s).

In some embodiments, one or more of the ribonucleotides of the mRNA molecule has been modified.

In some embodiments, one or more uracil bases has been modified, preferably to 5-methoxy-uridine.

In some embodiments, one or both caps of the mRNA molecule are synthetic, preferably a CleanCap®.

Overexpression is understood as “excessive” or higher levels of expression of a gene, such as that caused by increasing the frequency of transcription of a gene. Thus, it may also be viewed as above wild type or normal levels of expression. Overexpression can be defined with reference to the amount of protein produced for a population of cells, or by reference to a fold increase from wild type or normal levels of expression. Expression levels may be described as the amount of protein secreted per volume of cell culture. However, increased transcription may not necessarily lead to increased amounts of secreted protein.

A cell which overexpresses IL-10 and/or MMP9 comprises coding sequences expressing IL-10 and/or MMP9 at a higher level than in non-engineered cells. As described above, overexpression may be achieved through the introduction of exogenous nucleic acid encoding IL-10 and/or MMP9 such as mRNA, or genetic modification which stimulates expression of IL-10 and/or MMP9 from endogenous coding sequences. An engineered macrophage which overexpresses IL-10 and/or MMP9 may not necessarily secrete a greater amount of IL-10 and/or MMP9 than a non-engineered macrophage.

As used herein, over-expression relates to the artificial expression of a gene in increased quantity, relative to the expression level of the gene without the artificial modification, which may be referred herewith as wild-type or natural macrophage. As used in the Examples, expression levels were quantified at between 16 to 24 hours post-transfection. Expression levels as recited here are given for a population of macrophages at a concentration of 4×10⁶/ml (which equates to 2×10⁶ cells per cm²). In a preferred embodiment, over-expression is achieved via the introduction of an exogenous mRNA into the macrophage. In the Examples, the macrophages were transfected, isolated by centrifugation, re-suspended in TexMACs buffer supplemented with IL-3 and IL-14, and incubated at 37° C. under 5% CO2. Those skilled in the art would be aware of equivalent conditions suitable to determine secreted protein concentration. The following levels were determined experimentally under these conditions:

In an embodiment, overexpression of IL-10 means the secreted IL-10 protein level is greater than about 300 pg/ml. Suitably, the level of IL-10 expression is greater than about: 300 pg/ml or 400 pg/ml or 500 pg/ml or 600 pg/ml or 700 pg/ml or 800 pg/ml or 900 pg/ml or 1,000 pg/ml or 2,000 pg/ml or 3,000 pg/ml or 4,000 pg/ml or 5,000 pg/ml or 6,000 pg/ml or 7,000 pg/ml or 8,000 pg/ml or 9,000 pg/ml or 10,000 pg/ml or 11,000 pg/ml. In preferred embodiments, the secreted IL-10 protein level is greater than 10,000 μg/ml.

In an embodiment, “relative overexpression” of IL-10 in a culture of IL-10-engineered macrophages means the secreted IL-10 protein level in the culture is increased by about 100-300 pg/ml, or greater than about 300 pg/ml, relative to the average wild-type protein secretion of a culture of wild-type macrophages cultured under the same conditions. Suitably, the increase in the level of IL-10 expression is greater than about: 300 pg/ml or 400 pg/ml or 500 pg/ml or 600 pg/ml or 700 pg/ml or 800 pg/ml or 900 pg/ml or 1,000 pg/ml or 2,000 pg/ml or 3,000 pg/ml or 4,000 pg/ml or 5,000 pg/ml or 6,000 pg/ml or 7,000 pg/ml or 8,000 pg/ml or 9,000 pg/ml or 10,000 pg/ml or 11,000 pg/ml. In preferred embodiments, the secreted IL-10 protein level is greater than 10,000 pg/ml. Suitably, these IL-10 protein levels may be measured by culturing the macrophages as described above, wherein the concentration of macrophages in the medium is 4×10⁶ cells/ml, which equates to 2×10⁶ cells/cm², and measuring the concentration of the protein in the culture medium. Accordingly, the macrophage secretes IL-10 at a culture supernatant concentration of at least 10,000 pg/ml when cultured in vitro at a cell concentration of 4×10⁶/ml. In some embodiments, the engineered macrophage may secrete IL-10 at levels 1000 fold greater than non-engineered, non-polarised hMDMs. Non-engineered, non-polarised hMDMs are described in the art, for example in WO2019175595.

In an embodiment, expression of MMP9 means the secreted MMP9 protein level is between about 200 ng/ml and 2000 ng/ml. Suitably, the secreted MMP9 protein is greater than about: 300 ng/ml or 400 ng/ml or 500 ng/ml or 600 ng/ml or 700 ng/ml or 800 ng/ml or 900 ng/ml or 1,000 ng/ml. Suitably the secreted MMP9 protein level is between about 200 ng/ml and 2000 ng/ml. In preferred embodiments, the secreted MMP9 level is greater than 200 ng/ml. Suitably the engineered macrophage (comprising IL-10 and MMP9) has a secreted MMP9 protein level that is greater than the average level of secreted MMP9 protein in macrophages engineered with IL-10 alone. Suitably, the overall MMP activity of the engineered macrophage of the invention is also higher than the overall MMP activity of a macrophage engineered with IL-10 alone. In preferred embodiments, the overall MMP activity of the engineered macrophage is at least 1.5 times greater than an untransfected macrophage.

In an embodiment, “relative overexpression of MMP9” in a culture of MMP9-engineered macrophages means that the secreted MMP9 protein level in the culture is increased by between about 200 ng/ml and 2000 ng/ml, relative to the average wild-type protein expression of a culture of wild-type macrophages cultured under the same conditions. Suitably, the increase in the level of secreted MMP9 protein is greater than about: 300 ng/ml or 400 ng/ml or 500 ng/ml or 600 ng/ml or 700 ng/ml or 800 ng/ml or 900 ng/ml or 1,000 ng/ml, relative to the average wild-type protein expression of a culture of wild-type macrophages cultured under the same conditions. Suitably the increase in the level of secreted MMP9 protein level in the culture is between about 200 ng/ml and 2000 ng/ml, relative to the average wild-type protein expression of a culture of wild-type macrophages cultured under the same conditions. Suitably, these MMP9 protein levels may be measured—by culturing the macrophages as described above, wherein the concentration of macrophages in the medium is 4×10⁶ cells/ml, which equates to 2×10⁶ cells/cm², and measuring the concentration of the protein in the culture medium. Accordingly, the macrophage secretes MMP9 at a culture supernatant concentration of at least 200 ng/ml when cultured in vitro at a cell concentration of 4×10⁶/ml. In preferred embodiments, the secreted MMP9 level is greater than 200 ng/ml. Suitably the increase in the level of secreted MMP9 in a culture of IL-10-MMP9 engineered macrophages (i.e., comprising IL-10 and MMP9) is greater than the average increase in a culture of secreted MMP9 protein in macrophages engineered with MMP9 alone, relative to the average wild-type protein expression of a culture of wild-type macrophages cultured under the same conditions. Suitably, the overall MMP activity of the engineered macrophage of the invention is also higher than the overall MMP activity of a macrophage engineered with IL-10 alone. In some embodiments, these increases are synergistic. In preferred embodiments, the overall MMP activity of the engineered macrophage is at least 1.5 times greater than an untransfected macrophage.

In an embodiment, “relative underexpression of MMP9” in a culture of IL-10-engineered macrophages means that the secreted MMP9 protein level in the culture is decreased by between about 50 ng/ml and 100 ng/ml, between about 100 ng/ml and 200 ng/ml, between about 200 ng/ml and 2000 ng/ml, relative to the average wild-type protein expression of a culture of wild-type macrophages cultured under the same conditions. Suitably, the decrease in the level of secreted MMP9 protein is greater than about: 100 ng/ml or 300 ng/ml or 400 ng/ml or 500 ng/ml or 600 ng/ml or 700 ng/ml or 800 ng/ml or 900 ng/ml or 1,000 ng/ml, relative to the average wild-type protein expression of a culture of wild-type macrophages cultured under the same conditions. Suitably the decrease in the level of secreted MMP9 protein level in the culture is between about 200 ng/ml and 500 ng/ml, relative to the average wild-type protein expression of a culture of wild-type macrophages cultured under the same conditions.

The engineered macrophages of the present invention (comprising IL-10 and MMP9) secrete MMP9 at a level greater than the level of MMP9 expressed by macrophages engineered with IL-10 alone (See: FIG. 1: IL-10-MMP9 Trx vs IL-10 Trx). In one embodiment, the macrophages are engineered with at least one DNA vector encoding IL-10 and/or MMP9. It will be understood that a DNA vector may require one or more accessory sequences, such as a promoter, terminator, poly(A) signal sequence and the like.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

In one embodiment, the DNA vector may include one or more liver specific promoters or cirrhosis specific promoters. In some embodiments, the DNA vector may comprise a CX3CR1 promoter, an insulin-like growth factor 1 (IGF1), or a CD1 IB promoter.

“Operably linked” refers to an arrangement of elements wherein the components so described are configured so as to perform their usual function. Thus, a given promoter operably linked to a nucleic acid sequence is capable of affecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

‘treatment’ as used in the present invention means an intervention in a physiological condition which prevents, reduces, or removes the clinical symptoms associated with a given physiological condition in a subject.

A “therapeutically effective amount” of macrophages described in this specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g. animal model) systems.

By ‘subject’ or ‘individual’ or ‘animal’ or ‘patient’ is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

Suitably the subject may be in need of treatment. Suitably therefore the subject may have a disease, condition or disorder or be at risk of developing a disease, condition or disorder. Suitably the subject may display one or more symptoms of a disease, condition or disorder.

The engineered macrophages of the present invention may be for use in therapy. The macrophages of the present invention may be for use in treating an inflammatory condition and/or a fibrotic condition in a subject. As defined above, treating here can mean preventing, reducing or removing inflammation/fibrosis/organ damage. For example, the engineered macrophages maybe administered to a subject at an acute inflammation stage with the aim of preventing a chronic inflammatory condition. The engineered macrophages may also be administered to a subject at a chronic inflammation stage with the aim of preventing/reducing chronic fibrosis. The engineered macrophages may also administered to a subject who is experiencing an acute on chronic inflammatory state, such as acute on chronic liver failure (ACLF).

Suitably an acute disease or injury may be classed as a disease or injury with an onset of less than 24 weeks from cause. Suitably a chronic disease may be classed as a disease or injury which has persisted for more than 6 months. Suitably an acute-on chronic disease may be classed as a disease or injury with an onset of less than 24 weeks from cause in a patient that already has a chronic disease that has persisted for more than 6 months. Suitably, engineered macrophages of the present invention maybe administered to a subject with an acute occurrence to prevent transition to or increase of chronic inflammation and fibrosis.

Fibrosis refers to the deposition of extracellular matrix and connective tissue following tissue damage, which may result in replacing parenchymal tissue and eventually lead to scarring if in excess. In some embodiments, the condition is fibrosis.

In some embodiments, the condition is in or affects an organ selected from the group consisting of: a liver, lung, heart, kidney, pancreas, skin, gastrointestinal, bone marrow, hematopoietic tissue, nervous system, eye or a combination thereof.

Suitably, the condition is chronic organ damage associated with chronic inflammation. Suitably, the condition relates to the kidney, liver, or lung. For example, the condition maybe inflammatory liver damage, inflammatory kidney damage or inflammatory lung damage.

Suitably, the invention relates to a cell therapy product for inflammatory organ damage based on monocyte-derived macrophages genetically modified with payloads that induce a pro-restorative phenotype.

Suitably, the engineered macrophage has a pro-restorative phenotype and is anti-inflammatory and anti-fibrotic.

The M2-like phenotype is pro-restorative. Whereas the M1-like phenotype is pro-inflammatory.

The engineered macrophage includes exogenous coding sequence(s) for IL-10 and MMP9. This exogenous coding sequence may be provided in any suitable way. This may be in the form of a nucleic acid vector, howsoever delivered, or genetic modification. For example, using viral or non-viral vectors, DNA or RNA constructs, or by gene editing using any suitable technique.

The macrophage engineered to express IL-10 and MMP9 may be genetically engineered in any suitable way. For example, using viral or non-viral vectors, DNA or RNA constructs, or by gene editing using any suitable technique.

The macrophage may be virally engineered. In such methods of genetic engineering, the exogenous coding sequence/payload is introduced into the macrophage using a virus, such as a lentivirus, adenovirus or AAV. The virus may provide the exogenous coding sequence/payload as an “extrachromosomal” construct, or the gene may be integrated into the genome of the macrophage. Viral engineering of macrophages needs careful techniques, as documented in the art, to prevent the macrophages phagocytosing the viruses.

Suitably, the engineered macrophage is non-virally engineered, for example using nucleic acid vectors including the exogenous coding sequences. The nucleic acid may be any suitable nucleic acid, including DNA and RNA. Suitably the macrophage is transfected with a DNA vector. Suitably the DNA vector is a naked DNA vector, such that it is not associated with proteins and/or lipids. Suitably the DNA vector is not derived from a viral genome. Optionally, the DNA vector is a non-integrating vector such that it can function without integrating into the chromosomes of the macrophage. Alternatively, the nucleic acid vector is a messenger RNA (mRNA) molecule.

Suitably, the DNA vector comprises at least one sequence encoding IL-10 and/or MMP9, operably linked to a promoter.

As used herein, “encoding” refers to the ability of specific nucleotide sequences, such as a gene or an mRNA, to serve as templates for synthesis of macromolecules such as proteins in a cell. A gene encodes a protein if transcription and translation of mRNA corresponding to that gene produces the protein in a cell.

Suitably, the engineered macrophage is a genetically engineered macrophage comprising an nucleic acid construct overexpressing IL-10 and expressing MMP9.

Suitably, the mRNA molecule expresses IL-10 and/or MMP9. Alternatively worded, the mRNA includes the coding sequence for IL-10 and MMP9, which are exogenous to the macrophage. The mRNA may be chemically modified. Said chemical modification may be any suitable modification, most notably to improve the half-life of the mRNA in the cell. Suitable modifications are discussed extensively herein.

A nucleic acid vector can be introduced into cells using any suitable transfection method, such as but not limited to: cationic liposome-mediated transfection, lipofection, polymer encapsulation, peptide-mediated transfection, or biolistic particle delivery systems, such as “gene guns”. Suitably, the macrophage is transfected with nucleic acid via electroporation. Other suitable methods of transfection include nucleofection.

The macrophage may suitably be engineered by altering the genome of the macrophage by gene editing. Gene editing permits permanent insertion of the exogenous coding sequence. Numerous techniques of gene editing are known, including those that require the use of nucleases. Various nucleases are known that can be exploited to modify the genome, from base editing techniques, prime editing techniques to gene editing. Many nucleases are known—such as Zinc fingers, TALENs and guided nucleases. These may be guided by RNA (“RNA” guided nucleases)—such as the enzymes involved in CRISPR, including but not limited to Cas9, Cas12a, Cas13, Mad7 and the like. The macrophage itself may be subject to gene editing, or a progenitor cell may be gene edited prior to conversion to a macrophage.

Suitably, the macrophage is transfected with one or more free nucleic acids or vectors.

Suitably, the macrophage is transfected with one or more exogenous coding sequences for IL-10 and MMP9.

Suitably, the macrophage is provided with least one exogenous coding sequence for IL-10 and at least one exogenous coding sequence for MMP9.

Suitably, the macrophage is engineered to overexpress IL-10 and express MMP9.

Suitably, the macrophage is autologous or allogenic to the subject.

The invention also relates to a population of engineered macrophages as described herein. Said population may be for use as a cell therapy.

Suitably, the use comprises administering an effective amount of the engineered macrophage to the subject.

The invention also relates to a composition comprising the engineered macrophages of the invention or a population thereof. Suitably, the composition is a pharmaceutical composition.

Suitably, the engineered macrophages may be formulated into a pharmaceutical composition.

Suitably the composition is suitable for administration to a subject. Suitably the composition is a liquid.

Suitably the composition is an infusible liquid.

Suitably the engineered macrophages are for administration to a subject by any route. Delivery to a subject may be by local or systemic administration. In some embodiments, administration is by, e.g. local injection, nebulizer, systemic injection. Suitably the engineered macrophages are for administration to a subject by infusion. Suitably the engineered macrophages are for administration to a subject parenterally, suitably intravenously. Suitably the engineered macrophages are for administration to a subject by injection or infusion. Suitably the engineered macrophages are for administration to a subject intravenously by infusion.

The invention also relates to a method of improving the migration of monocytes to inflammation comprising the use of an engineered macrophage according to the first aspect of the invention, a population of macrophages according to the second aspect of the invention or a composition according to the third aspect of the invention.

Suitably the engineered macrophages, the engineered macrophages for use in therapy, or the engineered macrophages used in a method of improving the migration of monocytes to inflammation, have a chemoattractant effect on monocytes. The engineered macrophage may have a chemoattractant effect on monocytes in vitro or in vivo. Recruitment of host monocytes at the site of inflammation/damaged organ is advantageous for treating inflammatory conditions. In some embodiments, the engineered macrophages have a chemoattractant effect specifically on monocytes, and not other immune cell types. In some embodiments, the engineered macrophages localise to sites of fibrosis, and/or recruit monocytes to sites of fibrosis, when administered to a subject with fibrosis, such as a subject with a chronic inflammatory condition with a fibrotic element. Presence of the engineered macrophages or monocytes at sites of fibrosis may be determined by flow cytometry, as described in the Examples herein. In some embodiments, the site of fibrosis may be the lung or the liver.

### Producing an Engineered Macrophage Expressing IL-10 and MMP9

The invention also relates to a method of producing an engineered macrophage expressing IL-10 and MMP9 comprising transiently transfecting a macrophage with a combined mRNA construct comprising at least one sequence encoding IL-10 and at least one sequence encoding MMP9. In some embodiments, the macrophage is contacted with an anti-inflammatory treatment after transfection. In some embodiments, the macrophage is contacted with IL4 and IL13. In other embodiments, the macrophage is contacted with IL-4, IL-13 and M-CSF. Suitably the engineered macrophages overexpress IL-10 and have restored MMP activity as a result of co-expression with MMP9 (MMP activity is otherwise depressed by IL-10). Suitably the engineered macrophages are pro-restorative. Suitably the engineered macrophages produced can be used for cell therapy.

Notably, macrophages engineered to overexpress IL-10, or IL-10 and MMP9 display a pro-regenerative phenotype without further anti-inflammatory treatment following transfection. Suitably, the engineered macrophages produced are manufactured to a GMP-compliant standard. Suitably therefore the engineered macrophages and populations thereof are GMP-compliant.

In other embodiments, the method of producing an engineered macrophage comprises introducing a sequence encoding IL-10 and/or MMP9 into the genome of the macrophage.

In preferred embodiments, in a population of engineered macrophages produced by the method, at least 80% of the macrophages secrete IL-10.

Also provided herein is a method of improving cryoresilience in macrophages, comprising incubating the macrophages in medium comprising IL-4, IL-13 and M-CSF. According to some embodiments, the incubation with IL-4, IL-13 and M-CSF is prior to cryo-preservation. According to some embodiments, IL-4, IL-13 and M-CSF are removed from the media prior to cryopreservation, such that there is no IL-4, IL-13 and M-CSF in the cryopreservation medium. According to some embodiments, provided herein a method of cryopreserving macrophages, comprising incubating the macrophages in medium comprising IL-4, IL-13 and M-CSF prior to cryopreservation. According to some embodiments, the method for improving cryoresilience or the method for cryopreserving macrophages further comprises removing IL-4, IL-13 and M-CSF from the media prior to cryopreservation. As used herein, the term “cryoresillience” (can also be referred to as “recovery”) refers to the survival rate of macrophages following cryopreservation, optionally as measured by the percentage of viable cells post cryogenesis out of the macrophages that have been cryo-preserved. In some embodiments, the concentration of IL-4 and IL-13 in the medium are 20 ng/ml, the concentration of M-CSF is 100 ng/ml, and the macrophages are at a concentration of 4×10⁶ cells/ml. In some embodiments, the cells are incubated overnight in the medium comprising IL-4, IL-13 and M-CSF.

### Macrophages for Engineering

Suitably the macrophages for engineering have been produced from any suitable progenitor cell. Suitably the macrophages have been produced in vitro.

Suitably the macrophages for engineering are monocyte-derived. Suitably they are human monocyte derived macrophages (hMDM). Monocyte-derived means macrophages differentiated from monocytes. Monocytes are the natural precursors of macrophages and dendritic cells; they are contained in blood and bone marrow. Suitably the macrophages are derived from peripheral blood monocytes, suitably the macrophages are peripheral blood monocyte derived macrophages. Suitably the macrophages are human peripheral blood monocyte derived macrophages. Suitably the monocytes are isolated from a human subject.

Suitably the macrophages are derived from the monocytes by culturing the monocytes, suitably in vitro. Suitably the macrophages are derived from the monocytes by using any suitable culturing method.

Suitably the macrophages are produced in vitro from monocytes by a culturing method lasting between 3 to 8 days optionally 4 to 8 days. Suitably the macrophages are produced in vitro from monocytes by a culturing method lasting between 3 to 7 days, notably 4 to 7 days, or 5 to 7 days. In one embodiment, the macrophages are produced in vitro from monocytes by a culturing method that lasts 3-5 days, 4 or 5 days, or 7 days, known as a day 5 method or a day 7 method, respectively. One example of an in vitro method of producing macrophages from monocytes is described in WO2019/175595. The ‘day 5’ method is described in application number PCT/GB2021/051294 (the contents of which is herein incorporated by reference).

Suitably, the macrophages are produced by a ‘day 5’ method comprising:
(a) Culturing monocytes in medium for 3-5 or 4-5 days to produce macrophages, wherein the medium comprises one or more growth factors to stimulate macrophage production;
wherein step (a) takes place entirely in the same medium.

Suitably the medium comprises one or more growth factors selected from the CSF family, preferably M-CSF. Suitably the medium contains M-CSF at a concentration of between 25-150 ng/mL. Suitably the medium contains 100 ng/mL GMP-graded recombinant human macrophage colony-stimulating factor 1 (rhM-CSF-1; also known as ‘rh (recombinant human) CSF-1’).

In other embodiments, the macrophages have been produced from stem cells, such as pluripotent or multipotent stem cells. In particular embodiments, the macrophages have been produced from induced pluripotent stem cells (iPSCs).

### Anti-Inflammatory Treatment

In one embodiment, the method of producing an engineered macrophage, engineered with the combination of IL-10 and MMP9, comprises transient transfection of a macrophage with exogenous coding sequence(s) for IL-10 and MMP9. These coding sequences may be provided via transfection with a nucleic acid, for example one or more mRNA molecules.

The macrophage is provided with least one exogenous coding sequence for IL-10 and at least one exogenous coding sequence for MMP9.

In some embodiments, the macrophage is contacted with an anti-inflammatory treatment after transfection with an exogenous coding sequence(s). Suitably the anti-inflammatory treatment comprises anti-inflammatory cytokines. In some embodiments the macrophage is contacted with an anti-inflammatory treatment comprising IL4 and IL13.

In some embodiments, the anti-inflammatory treatment may be added during the method of transfecting the macrophages.

The transfected macrophages may be contacted with these anti-inflammatory cytokines IL4 and IL13) for a period of about 2 hours to about 48 hours, suitably 4 hours to 40 hours, suitably 12 to 24 hours, optionally around 16 hours.

The transfected macrophages may be contacted with these anti-inflammatory cytokines (IL4 and IL13) at a concentration of between 2 ng/mL and 200 ng/mL, suitably between 5 ng/mL and 150 ng/mL, suitably between 10 ng/mL to 100 ng/mL, suitably between 15 ng/mL to 75 ng/mL, suitably between 20 ng/mL to 50 ng/mL.

Suitably the anti-inflammatory treatment is used as a solution.

For the step of contacting the macrophage with IL4+IL13, suitably cells are plated as follows: 2×10⁶ hMDMs/cm² at 4×10⁶/mL.

The invention will now be described with reference to the following figures.

### FIGURES

FIG. 1— Plots depicting experimental results of transfecting macrophages with IL-10 and IL-10+MMP9—the macrophages secrete high levels of IL-10. A: IL-10 transfected hMDMs show decrease level of MMP9 expression. B: IL-10+MMP9 transfected hMDMs show increased MMP9 levels. The dotted line represents the minimal desired level for the product.

FIG. 2— Plots depicting experimental results of transfecting macrophages with various constructs. Depicted are the percentage of IL-10 secreting cells (%) over a 2 hour time frame using flow cytometry capture assay. Both construct delivers high percentage of secreting cells. Each symbol represents an independent donor.

FIG. 3— Plots depicting experimental results of transfecting macrophages with various constructs in terms of cell surface markers. Flow cytometry analysis of identity cell surface markers for hMDMs by flow cytometry. Black solid line is the normalised level of expression on the non-engineered cells. Each plot represents a different macrophage cell surface marker: A: CD45, B: CD14, C: CD206; D: CCR2, E: CD163, F: CD169 and G: 25F9.

FIG. 4— Plots depicting experimental results of transfecting macrophages with various constructs in terms of pro-inflammatory markers. Pro-inflammatory markers CD86 (A) and HLA-DR (B) are significantly downregulated in engineered cells, as measured by flow cytometry. Other inflammatory markers are substantially unchanged vs non-Trx hMDMs. CD80 expression is desirably no more than 20% or 10-15% above non-genetic engineered (NTx) levels. The solid black line is the levels of non-Trx cells. The dotted red line represents the maximum desired level. Each symbol represents an independent donor.

FIG. 5— Plot depicting experimental results of transfecting macrophages with various constructs in terms of phagocytic ability. Phagocytosis is measured using pH-sensitive (pHrodo) beads coated with E. coli. Percentage of phagocytosing macrophages is measured by flow cytometry. Each symbol represents an independent donor. The dotted line represents the minimal desired percentage of phagocytic macrophages.

FIG. 6— Plots depicting experimental results of transfecting macrophages with various constructs in terms of M1 (A and B) and M2 markers (C and D). M1 and M2 markers are used as indicators of pro-inflammatory and anti-inflammatory phenotypes for in vitro generated macrophages. Flow cytometry analysis of M1-type and M2-type cell surface markers in macrophages treated overnight with supernatants from Non-Trx, Non+Trx+Treatment, IL-10 Trx and IL-10+MMP9 Trx hMDMs. M2=positive control: macrophages from the same donor polarized using high levels of IL-10 (dotted line). Plot A shows the results for CD86, and Plot B for HLA DR. Plot C shows the results for CD206 and plot D for CD163.

FIG. 7— Plots depicting experimental results of transfecting macrophages with various constructs in terms of effect on migration of other cells. PBMCs migration assay results as measured by flow cytometry. Only monocytes displayed significant migration. Data are normalised to Ntrx cells (black line) and the minimal desired increase is indicated by the dotted line.

FIG. 8— Plots depicting experimental results of transfecting macrophages with various constructs on the expression of MMPs. Shown is MMP activity assay in supernatants of NTrx, NTrx+Treatment, IL-10 Trx, IL-10+MMP9 Trx hMDMs tested. The dotted line represents 1.5× times the level of activity measured in supernatants of NTrx hMDMs. Each symbol represents an independent donor.

FIG. 9—Plots depicting experimental results of transfecting macrophages with various constructs on location of the site of damage in vivo. Shown are the percentage (%) of live (7AAD−) human macrophages measured in digests from fibrotic livers using flow cytometry. Each dot is a distinct mouse. Data are reported as mean±standard deviation (SD). Plot A is localisation to lung and Plot B is localisation to liver.

FIG. 10—Plots depicting experimental results of transfecting macrophages with various constructs on expression of IL-10 and MMP9 in vivo. Shown is the measurement of human IL-10 and human MMP9 by ELISA in the circulation of mice with chronic liver fibrosis at distinct time points after cell injection. Each dot is a distinct mouse. Data is reported as average±SD. Plot A is IL-10 in the plasma and plot B is MMP9 in the plasma.

FIG. 11—Plots depicting experimental results of transfecting macrophages with various constructs after injection into mouse models. Mouse inflammatory cytokines IL1b and TNFa were measured in plasma by ELISA to verify inflammation elicited, if any, by injecting engineered and non-engineered cells. Measurements were conducted at various time points after cell injection. Black dotted line represents maximum tolerated levels. Each dot is a distinct mouse. Data are reported as average±SD. Plot A depicts the results for mIL-1β and plot B depicts the results for mTNF-α.

FIG. 12—Recruitment of human monocyte by conditioned media from non-engineered hMDMs or hMDMs transfected with various genes, as indicated. The assay was conducted as indicated in material and methods, and results analysed by flow cytometry. Every symbol represents a conditioned medium from an independent donor. Data are reported as single donor dispersion, average and standard deviation.

FIG. 13—Conditioned medium from hMDMs transfected with IL-10 specifically recruits monocytes. The data shown was generated using the same protocol as in FIG. 12, but shows the migration of individual cell types. Every symbol represents a conditioned medium from an independent donor. Data are reported as single donor dispersion, average and standard deviation.

FIG. 14—Conditioned medium from IL-10 transfected macrophages polarise unpolarised macrophages towards a pro-restorative phenotype. The assay was conducted as indicated in part (A)—macrophages at day 5 of culture (culture method as described in the Example) were treated with conditioned medium from non-engineered cells treated with IL-4, IL-13 and M-CSF, cells engineered with a payload of either IL-10 or MERTK, or polarisation medium (comprising MexMACS and IL-10 at 50 ng/ml). The expression of HLA-DR (B), CD86 (C), 25F9 (D), CD206 (E) and CD163 (F) were assessed using flow cytometry as described in the Examples.

FIG. 15—Conditioned medium from IL-10 transfected macrophages rescue pro-inflammatory macrophages and promote a pro-restorative phenotype. The assay was conducted as indicated in part (A)—macrophages at day 5 of culture (culture method as described in the Example) were treated with conditioned medium from non-engineered cells treated with IL-4, IL-13 and M-CSF, cells engineered with a payload of either IL-10 or MERTK, or polarisation medium (comprising MexMACS and IL-10 at 50 ng/ml). The expression of HLA-DR (B), CD86 (C), 25F9 (D), CD206 (E) and CD163 (F) were assessed using flow cytometry as described in the Examples.

FIG. 16—Macrophages transfected with IL-10 alone have a comparable secretome to non-engineered, non-polarised macrophages. Cells were treated as set out in the figure: untransfected cells, either with (UT TR) or without (UT) post-transfection treatment with IL-4, IL-13 and M-CSF, or transfected with the specified payload (IL-10, MERTK, MMP9 or MMP12 as indicated on the x axis). Concentration of IL-10 (A), IL-6 (B), CXCL8 (C), IL-12p70 (D), IL-2 (E), IL-1β (F), TNF-α (G) and IFN-Y (H) were measured in culture supernatants, wherein the cells were cultured at a concentration of 4×10⁶ cells/ml, as described herein.

FIG. 17—Macrophages transfected with IL-10 alone localise to the liver following administration, to mice with a liver fibrosis model. IL-10 transfected cells or PBS was administered intravenously to mice with a CCl₄-induced model of fibrotic liver injury, similarly to as described in Example 8.

FIG. 18—MMP9 transfected macrophages exhibit increased MMP activity. Total MMP activity was measured as described in the Examples in untransfected macrophages, with or without the administration an inhibitor of Stimulator of interferon Genes (iSTING), or macrophages transfected with the specified payloads. Data presented as mean±SD. *** p≤0.005.

FIG. 19— MMP9 transfected macrophages have increased expression of other matrix metalloproteinases. The expression of MMP1, 3, 7, 8 and 10 were measured in macrophages which were untransfected (NT), with or without the administration a STING inhibitor (STINGi), or transfected with MMP9 alone. Darker shading represents greater expression.

FIG. 20—RTX001 macrophages recruit monocytes in vitro. RTX001 macrophages were produced by transfection with the bicistronic construct with a sequence as set forth in SEQ ID NO: 16. PBMCs migration assay was conducted as described in the Examples and results as measured by flow cytometry. Results are normalised to untransfected (NTrx) cells.

FIG. 21—RTX001 macrophages recruit monocytes in vivo. The recruitment of total myeloid cells was assessed using flow cytometry to detect CD11b+Tim4− cells as a proportion of total CD45+ cells (left panel). Monocytes were identified as Ly6Chi CD64− CD45+ cells by flow cytometry (right panel). Recruitment was measured in a mouse CCl4-induced model of liver fibrosis. RTX001 transfected macrophages, untransfected macrophages or a PBS vehicle were injected into the mouse, and recruitment assessed 24 hours after administration.

FIG. 22—RTX001 macrophages promote an anti-inflammatory environment in vivo. Murine IL-10 in liver homogenate were measured 24 hours after RTX001 macrophages, untransfected macrophages or PBS control were administered to a CCL4-induced mouse model of liver fibrosis.

FIG. 23—Macrophages engineered to express IL-10 alone, and IL-10 and MMP9, exhibit similar pro-restorative secretomes. The amount of secreted IL-2 (A), IL-12p70 (B), IFNg (C), TNF-a (D) and IL1B (E) was measured in supernatants in culture medium, wherein the engineered cells were cultured at a concentration of 4×10⁶ cells/ml.

FIG. 24—Administration of RTX001 macrophages reduced scar forming cells in vivo. RTX001 macrophages, untransfected macrophages or PBS vehicle were administered to a CCL4-induced mouse model of liver fibrosis. Histological sections were taken from mice 1 weeks after administration and stained for a-SMA (Left panel). The percentage area in the sections positive for a-SMA was enumerated, with the results shown in the right panel.

FIG. 25. RTX001 macrophage CM significantly reduced LX-2 60 SMA expression. (A) Fold change in αSMA mean fluorescent intensity (MFI) relative to the TGF-β stimulated control (no CM). (B) Graph comparing percentage of cells expressing αSMA. Experimental triplicates were averaged and data presented as mean±SD (n=1). ** p<0.01.

FIG. 26—Optimised bicistronic mRNA results in improved pro-restorative properties. Cells were transfected with a non-optimised mRNA (SEQ ID NO: 2, further comprising a polyA tail 120 residues long), or optimised mRNA (SEQ ID NO: 16), both of which are bicistronic mRNAs encoding both IL-10 and MMP9. The amount of secreted MMP9 (left panel) and IL-10 (right panel) in culture supernatants was detected, and MMP activity measured as described herein. MMP activity was normalised to untransfected cells.

FIG. 27—RTX001 is stable in an inflammatory environment. The change in CD80 and CD86 (inflammatory markers) and CD206 (pro-restorative marker and general macrophage identity marker) was measured in engineered macrophages exposed to IFN-γ at the specified concentrations. No significant change was seen in proinflammatory markers (CD80 and CD86) or macrophage identity marker CD206 (A). MATCH-like (Ntrx) and RTX0001 (Trx+tr) cells were incubated with IFN-γ (1.05, 10.5 or 105 ng/mL) for 24 h. MFI of proinflammatory markers HLA-DR and CD80 was assessed using flow cytometry. Data shown is the fold change vs Ntrx cells cultured without IFN-γ (B and C).

FIG. 28. Table showing the expression of macrophage markers, or secretion of cytokines, in the specified macrophage products. The macrophage products consist of: non-transfected and untreated (UT) or treated (UT+TR) with IL-4, IL-13 and M-CSF; transfected with IL-10 and MMP9 and untreated (IL-10 MMP9 TRx) or treated with IL-4, IL-13 and M-CSF post-transfection (IL-10 MMP9 TRx+TR). Mean Fluorescence Intensity was measured by flow cytometry as described herein.

FIG. 29—post-transfection treatment does not contribute to further polarisation of the engineered macrophages. The expression of cD86 (left) and MHC II (right) were detected in macrophage products by flow cytometry. The macrophage products tested consisted of: untransfected macrophages without (NTx) or with treatment with IL-4, IL-13 and M-CSF (NTx+TR); macrophages transfected with both MMP9 and IL-10, without (IL-10-MMP9) or with further treatment with IL-4, IL-13 and M-CSF post-transfection (IL-10-MMP9+TR).

FIG. 30—Treatment with IL-4, IL-13 and M-CSF improved cryoresilience. Viability of macrophage product populations was determined following cryopreservation. The macrophage products consisted of: untransfected macrophages without (NTrx) or with further IL-4, IL-13 and M-CSF treatment (NTrx+Tr); macrophages transfected with both IL-10 and MMP9 without (Trx) or with further IL-4, IL-13 and M-CSF treatment post-transfection (Trx+Tr). Viability was assessed by measuring the cells still viable post cryopreservation as a proportion of the total cells viable pre-cryopreservation.

FIG. 31— Summary of the protocol used to measure efficacy in a mouse-on-mouse model in Example 25.

FIG. 32—schematic summarising the mechanisms which are the thought to be the basis for the therapeutic efficacy of the engineered macrophages.

The data shown in these figures demonstrates that the inventors have shown that the surprising and effective combination of the expression of two specific genes in tandem, IL-10 and MMP9, has an unforeseen and surprising benefit to the phenotype of the engineered macrophage. That the expression of MMP9 alone could rescue the dampened expression/activity of the MMPs seen in IL-10 transfected macrophages is astonishing. The effect appears to be synergistic, as shown above. Further, the excellent recruitment of monocytes by the combined overexpression was not predicted, as convention dictates that entities such as cytokines are involved in the recruitment process.

The genetic engineering of a macrophage to permit overexpression of IL-10 in combination with MMP9 delivers a number of features and functions desirable for a cell therapy, which is useful in inflammatory conditions such as for organ damage regeneration. The inventors have shown that IL-10 in combination of MMP9 delivers:
A solid macrophage identity, not perturbed by the engineering process.
A strong anti-inflammatory phenotype.
Ability to pattern naïve macrophages towards a pro-restorative phenotype.
Excellent phagocytic capacity.
Reassuring safety and biodistribution profile, including infiltration in the damaged organ and rapid clearance/absence in other organs.

The above features are shared with macrophages engineered with IL-10 alone previously investigated by the inventors. However, the combination of IL-10 and MMP9 delivers some specific and surprising features, key to deliver a desired therapeutic effect, such as:
A strong ability to attract monocytes, to then be patterned to a pro-restorative phenotype.
Ability to restore the MMP activity (surrogate for fibrosis/extracellular matrix (ECM) remodelling) abrogated by the engineering of IL-10 alone.

Therefore, the inventors believe that engineering a macrophage with a combination of IL-10 and MMP9 will deliver an efficacious product able to have both anti-inflammatory and anti-fibrotic functions in therapy, for example several organ damage settings, both acute and chronic. Remodelling of ECM components is paramount in acute damage settings to ensure the restitutio ad integrum of the tissue and proper regeneration.

### EQUIVALENTS

Those skilled in the art will recognise or be able to ascertain using no more than routine experimentation, equivalents of the specific embodiments described herein. Such equivalents are intended to be encompassed by the following claims. Any combination of the embodiments disclosed in the any plurality of the dependent claims or Examples is contemplated to be within the scope of the disclosure.

### INCORPORATION BY REFERENCE

The disclosure of each and every patent, patent application publication, and scientific publication referred to herein is specifically incorporated herein by reference in its entirety, as are the contents of its Figures.

### REFERENCES

1 Williams, R. et al. Addressing liver disease in the UK: a blueprint for attaining excellence in health care and reducing premature mortality from lifestyle issues of excess consumption of alcohol, obesity, and viral hepatitis. Lancet 384, 1953-1997, doi:10.1016/S0140-6736(14)61838-9 (2014).
2 Pellicoro, A., Ramachandran, P., Iredale, J. P. & Fallowfield, J. A. Liver fibrosis and repair: immune regulation of wound healing in a solid organ. Nat Rev Immunol 14, 181-194, doi:10.1038/nri3623 (2014).
3 Fallowfield, J. A. Future mechanistic strategies for tackling fibrosis—an unmet need in liver disease. Clin Med (Lond) 15 Suppl 6, s83-87, doi:10.7861/clinmedicine.15-6-s83 (2015).
4 Forbes, S. J. & Newsome, P. N. Liver regeneration—mechanisms and models to clinical application. Nat Rev Gastroenterol Hepatol, doi:10.1038/nrgastro.2016.97 (2016).
5 Thomas, J. A., Ramachandran, P. & Forbes, S. J. Studies of macrophage therapy for cirrhosis—From mice to men. J Hepatol 68, 1090-1091, doi:10.1016/j.jhep.2017.11.043 (2018).
6 Forbes, S. J., Gupta, S. & Dhawan, A. Cell therapy for liver disease: From liver transplantation to cell factory. J Hepatol 62, S157-169, doi:10.1016/j.jhep.2015.02.040 (2015).
7 Falasca, L., Bergamini, A., Serafino, A., Balabaud, C. & Dini, L. Human Kupffer cell recognition and phagocytosis of apoptotic peripheral blood lymphocytes. Exp Cell Res 224, 152-162, doi:10.1006/excr.1996.0123 (1996).
8 Ramachandran, P. et al. Differential Ly-6C expression identifies the recruited macrophage phenotype, which orchestrates the regression of murine liver fibrosis. Proc Natl Acad Sci USA 109, E3186-3195, doi:10.1073/pnas.1119964109 (2012).
9 Wynn, T. A. & Barron, L. Macrophages: master regulators of inflammation and fibrosis. Semin Liver Dis 30, 245-257, doi:10.1055/s-0030-1255354 (2010).
10 Zigmond, E. et al. Infiltrating monocyte-derived macrophages and resident kupffer cells display different ontogeny and functions in acute liver injury. J Immunol 193, 344-353, doi:10.4049/jimmunol.1400574 (2014).
11 Campana, L., Esser, H., Huch, M. & Forbes, S. Liver regeneration and inflammation: from fundamental science to clinical applications. Nat Rev Mol Cell Biol 22, 608-624, doi:10.1038/s41580-021-00373-7 (2021).
12 Thomas, J. A. et al. Macrophage therapy for murine liver fibrosis recruits host effector cells improving fibrosis, regeneration, and function. Hepatology 53, 2003-2015, doi:10.1002/hep.24315 (2011).
13 Moore, J. K. et al. Phenotypic and functional characterization of macrophages with therapeutic potential generated from human cirrhotic monocytes in a cohort study. Cytotherapy 17, 1604-1616, doi:10.1016/j.jcyt.2015.07.016 (2015).
14 Fraser, A. R. et al. Development, functional characterization and validation of methodology for GMP-compliant manufacture of phagocytic macrophages: A novel cellular therapeutic for liver cirrhosis. Cytotherapy 19, 1113-1124, doi:10.1016/j.jcyt.2017.05.009 (2017).
15 Starkey Lewis, P. J., Moroni, F. & Forbes, S. J. Macrophages as a Cell-Based Therapy for Liver Disease. Semin Liver Dis 39, 442-451, doi:10.1055/s-0039-1688502 (2019).
16 Moroni, F. et al. Safety profile of autologous macrophage therapy for liver cirrhosis. Nat Med 25, 1560-1565, doi:10.1038/s41591-019-0599-8 (2019).
17 Das, S. T. et al. Monomeric and dimeric CXCL8 are both essential for in vivo neutrophil recruitment. PLoS One 5, e11754, doi:10.1371/journal.pone.0011754 (2010).
18 Wilgus, T. A., Roy, S. & McDaniel, J. C. Neutrophils and Wound Repair: Positive Actions and Negative Reactions. Adv Wound Care (New Rochelle) 2, 379-388, doi:10.1089/wound.2012.0383 (2013).
19 Lin, Z. Q., Kondo, T., Ishida, Y., Takayasu, T. & Mukaida, N. Essential involvement of IL-6 in the skin wound-healing process as evidenced by delayed wound healing in IL-6-deficient mice. J Leukoc Biol 73, 713-721, doi:10.1189/jlb.0802397 (2003).
20 Wynn, T. A. & Vannella, K. M. Macrophages in Tissue Repair, Regeneration, and Fibrosis. Immunity 44, 450-462, doi:10.1016/j.immuni.2016.02.015 (2016).
21 Mantovani, A. et al. The chemokine system in diverse forms of macrophage activation and polarization. Trends Immunol 25, 677-686, doi:10.1016/j.it.2004.09.015 (2004).
22 Mosser, D. M. & Edwards, J. P. Exploring the full spectrum of macrophage activation. Nat Rev Immunol 8, 958-969, doi:10.1038/nri2448 (2008).

23 Acharya, D., Li, X. R. L., Heineman, R. E. & Harrison, R. E. Complement Receptor-Mediated Phagocytosis Induces Proinflammatory Cytokine Production in Murine Macrophages. Front Immunol 10, 3049, doi:10.3389/fimmu.2019.03049 (2019).

24 Dreschers, S. et al. Metalloproteinases TACE and MMP-9 Differentially Regulate Death Factors on Adult and Neonatal Monocytes After Infection with Escherichia coli. Int J Mol Sci 20, doi:10.3390/ijms20061399 (2019).
25 Mosser, D. M. & Zhang, X. Interleukin-10: new perspectives on an old cytokine. Immunol Rev 226, 205-218, doi:10.1111/j.1600-065X.2008.00706.x (2008).
26 Riley, J. K., Takeda, K., Akira, S. & Schreiber, R. D. Interleukin-10 receptor signaling through the JAK-STAT pathway. Requirement for two distinct receptor-derived signals for anti-inflammatory action. J Biol Chem 274, 16513-16521, doi:10.1074/jbc.274.23.16513 (1999).
27 Campana, L. et al. The STAT3-IL-10-IL-6 Pathway Is a Novel Regulator of Macrophage Efferocytosis and Phenotypic Conversion in Sterile Liver Injury. J Immunol 200, 1169-1187, doi:10.4049/jimmunol.1701247 (2018).
28 Braat, H., Peppelenbosch, M. P. & Hommes, D. W. Interleukin-10-based therapy for inflammatory bowel disease. Expert Opin Biol Ther 3, 725-731, doi:10.1517/14712598.3.5.725 (2003).
29 Nelson, D. R., Lauwers, G. Y., Lau, J. Y. & Davis, G. L. Interleukin 10 treatment reduces fibrosis in patients with chronic hepatitis C: a pilot trial of interferon nonresponders. Gastroenterology 118, 655-660, doi:10.1016/s0016-5085(00)70134-x (2000).
30 Marlow, G. J., van Gent, D. & Ferguson, L. R. Why interleukin-10 supplementation does not work in Crohn's disease patients. World J Gastroenterol 19, 3931-3941, doi:10.3748/wjg.v19.i25.3931 (2013).
31 lyer, S. S. & Cheng, G. Role of interleukin 10 transcriptional regulation in inflammation and autoimmune disease. Crit Rev Immunol 32, 23-63, doi:10.1615/critrevimmunol.v32.i1.30 (2012).
32 Hung, K. S. et al. Interleukin-10 gene therapy reverses thioacetamide-induced liver fibrosis in mice. Biochem Biophys Res Commun 336, 324-331, doi:10.1016/j.bbrc.2005.08.085 (2005).
33 Chou, W. Y. et al. Electroporative interleukin-10 gene transfer ameliorates carbon tetrachloride-induced murine liver fibrosis by MMP and TIMP modulation. Acta Pharmacol Sin 27, 469-476, doi:10.1111/j.1745-7254.2006.00304.x (2006).
34 Huang, Y. H. et al. Therapeutic effect of interleukin-10 on CCl4-induced hepatic fibrosis in rats. World J Gastroenterol 12, 1386-1391, doi:10.3748/wjg.vl2.i9.1386 (2006).
35 Zhang, L. J., Zheng, W. D., Shi, M. N. & Wang, X. Z. Effects of interleukin-10 on activation and apoptosis of hepatic stellate cells in fibrotic rat liver. World J Gastroenterol 12, 1918-1923, doi:10.3748/wjg.v12.i12.1918 (2006).
36 Cypel, M. et al. Functional repair of human donor lungs by IL-10 gene therapy. Sci Transl Med 1, 4ra9, doi:10.1126/scitranslmed.3000266 (2009).
37 Cua, D. J., Hutchins, B., LaFace, D. M., Stohlman, S. A. & Coffman, R. L. Central nervous system expression of IL-10 inhibits autoimmune encephalomyelitis. J Immunol 166, 602-608, doi:10.4049/jimmunol.166.1.602 (2001).
38 Starkey Lewis, P. et al. Alternatively activated macrophages promote resolution of necrosis following acute liver injury. J Hepatol 73, 349-360, doi:10.1016/j.jhep.2020.02.031 (2020).
39 Ma, P. F. et al. Cytotherapy with M1-polarized macrophages ameliorates liver fibrosis by modulating immune microenvironment in mice. J Hepatol 67, 770-779, doi:10.1016/j.jhep.2017.05.022 (2017).
40 Wilson, H. M. et al. Bone-marrow-derived macrophages genetically modified to produce IL-10 reduce injury in experimental glomerulonephritis. Mol Ther 6, 710-717, doi:10.1006/mthe.2002.0802 (2002).
41 Boehler, R. M. et al. Lentivirus Delivery of IL-10 to Promote and Sustain Macrophage Polarization Towards an Anti-Inflammatory Phenotype. Biotechnol Bioeng. 2014 June; 111(6): 1210-1221. doi:10.1002/bit.25175 (2014).
42 D'Amico et al, Further decompensation as a new prognostic stage in cirrhosis. Results of a large multicenter cohort study supporting Baveno VII statements. Journal of Hepatology 78(S1) S105-106. (2023)
43 Trebicka J, et al. International Variceal Bleeding Observational Study Group and Baveno Cooperation. Rebleeding and mortality risk are increased by ACLF but reduced by pre-emptive TIPS. J Hepatol; 73(5):1082-1091. (2020)
44 Starkey Lewis P J, Moroni F, Forbes S J. Macrophages as a Cell-Based Therapy for Liver Disease. Semin Liver Dis., 39(4):442-451. (2019)
45 Cabrera S, Gaxiola M, Arreola J L, Ramírez R, Jara P, D'Armiento J, Richards T, Selman M, Pardo A. Overexpression of MMP9 in macrophages attenuates pulmonary fibrosis induced by bleomycin. Int J Biochem Cell Biol; 39(12):2324-38. (2007)
46 Moreau R, et al. CANONIC Study Investigators of the EASL-CLIF Consortium. Acute-on-chronic liver failure is a distinct syndrome that develops in patients with acute decompensation of cirrhosis. Gastroenterology, 144(7):1426-37 1437.e1-9. (2013)
47 Fan, Y. Y., Ding, W., Zhang, C., Fu, L., Xu, D. X., & Chen, X. Obeticholic acid prevents carbon tetrachloride-induced liver fibrosis through interaction between farnesoid X receptor and Smad3. International immunopharmacology, 77, 105911 (2019)
48 Younossi, Z. M., Ratziu, V., Loomba, R., Rinella, M., Anstee, Q. M., Goodman, Z., Bedossa, P., Geier, A., Beckebaum, S., Newsome, P. N., Sheridan, D., Sheikh, M. Y., Trotter, J., Knapple, W., Lawitz, E., Abdelmalek, M. F., Kowdley, K. V., Montano-Loza, A. J., Boursier, J., Mathurin, P., . . . REGENERATE Study Investigators. Obeticholic acid for the treatment of non-alcoholic steatohepatitis: interim analysis from a multicentre, randomised, placebo-controlled phase 3 trial. Lancet (London, England), 394(10215), 2184-2196. (2019)
49 Wettstein, G., Luccarini, J. M., Poekes, L., Faye, P., Kupkowski, F., Adarbes, V., Defrêne, E., Estivalet, C., Gawronski, X., Jantzen, I., Philippot, A., Tessier, J., Tuyaa-Boustugue, P., Oakley, F., Mann, D. A., Leclercq, I., Francque, S., Konstantinova, I., Broqua, P., & Junien, J. L. The new-generation pan-peroxisome proliferator-activated receptor agonist IVA337 protects the liver from metabolic disorders and fibrosis. Hepatology communications, 1(6), 524-537 (2017)
50 Francque, S. M., Bedossa, P., Ratziu, V., Anstee, Q. M., Bugianesi, E., Sanyal, A. J., Loomba, R., Harrison, S. A., Balabanska, R., Mateva, L., Lanthier, N., Alkhouri, N., Moreno, C., Schattenberg, J. M., Stefanova-Petrova, D., Vonghia, L., Rouzier, R., Guillaume, M., Hodge, A., Romero-Gómez, M., . . . NATIVE Study Group. A Randomized, Controlled Trial of the Pan-PPAR Agonist Lanifibranor in NASH. The New England journal of medicine, 385(17), 1547-1558 (2021)

### EXAMPLES

### Materials and Methods

The following protocols were used to generate the data described in the following examples.

### Macrophages Cell Culture

We isolated monocytes from a buffy coat product from a healthy volunteer sourced from the Scottish National Blood Transfusion Service (SNBTS) using a Ficoll gradient (GE Healthcare) followed by a magnetic column selection using CliniMACS CD14 Reagent (Miltenyi Biotec). We then matured monocytes for 1 to 7 days in culture in TexMACS without phenol red (Miltenyi Biotec) in the presence of 100 ng/mL GMP-graded recombinant human macrophage colony-stimulating factor (rhM-CSF) (R&D System, Biotechne). hMDMs are cultured in 6 wells multi-well plate (Corning Costar) at a density of 2×10⁶/cm² for five days. hMDMs were counted using an automated counter (TC20, BioRad).

### Macrophage Engineering

Small/Medium Scale Transfection—mRNA

Pellet mature macrophages at 300*g, 5 min, then remove supernatant and resuspend cells at density 50*10\hat{ }6 or 100*10\hat{ }6 cells/ml in supplemented buffer P3 (Lonza). Add mRNA at concentration 2 ug/10\hat{ }6 cells for IL10 (798 NT) or 8 ug/10\hat{ }6 cells for IL10-MMP9 (2985 NT) and mix well by pipetting. Transfer the cell suspension to an electroporation cuvette/cassette (100 ul/1 ml). Transfect the cells on Lonza Nucleofector using pulse code CM-137. Collect the cells in a sterile Falcon tube. Wash the electroporation cuvette/cassette with 100 ul/1 ml TexMACS medium supplemented with M-CSF (100 ng/ml), IL4 (20 ng/ml) and IL13 (20 ng/ml) and add to the cell suspension. Place the cells in a TC incubator for 20 min. Perform a cell count and adjust cell density to 4*10\hat{ }6/ml. Seed cells at density 2*10\hat{ }6/cm² and place in cell culture incubator.

Large Scale Fluidics Transfection—mRNA

Pelleted mature macrophages at 300*g, 5 min. Removed supernatant and re-suspended cells at density 200*10\hat{ }6 cells/ml in supplemented buffer P3 (Lonza). Transferred mRNA at concentration 2 ug/10\hat{ }6 cells for IL10 (798 NT) or 8 ug/10\hat{ }6 cells for IL10-MMP9 (2985 NT) to a sterile Falcon tube and topped up with supplemented buffer P3 to a volume equal to the cell suspension. Set up Lonza Nucleofector LV transfection. Transferred the cell suspension and the mRNA to a 4D-Nucleofector LV Reservoir as appropriate. Attached a cell culture bag pre-filled with TexMACS medium supplemented with M-CSF (100 ng/ml), IL4 (20 ng/ml) and IL13 (20 ng/ml) to the tubing of the electroporation cassette to elute the cells in. Transfected the cells on Lonza Nucleofector using pulse code CM-137. Transferred the bag with cells to a TC incubator for 20 min. Performed a cell count and adjust cell density to 4*10\hat{ }6/ml. Seeded cells at density 2*10\hat{ }6/cm² and placed in cell culture incubator.

Small Scale Transfection—pDNA

‘ Payload’ as used herein is a gene or genes of therapeutic interest, which is introduced via transfection to test its effect on macrophages.

Day 5 human monocyte-derived macrophages (hMDMs) were resuspended in CliniMACS® Electroporation Buffer (Miltenyi Biotec, #170-076-625) at density 75*10\hat{ }6 or 150*10\hat{ }6 cells/ml and 100-300 μl suspension was transferred to an electroporation cuvette with 0.2 cm gap size. 5 pg plasmid DNA per 5*10\hat{ }6 cells were added directly into the cuvette and mixed with the cells by gently flicking the cuvette. The cells were transfected using CliniMACS® Electroporator controlled by CliniMACS Prodigy®. The parameters for electroporation are outlined in Table 1 below (as further described in Patent Application PCT/GB2021/051300, published as WO2021240167):

TABLE 1
Parameters for transfection by electroporation of IL10-expressing plasmid in hMDMs.

| 1st pulse | | | | | 2nd pulse | | | |
|---|---|---|---|---|---|---|---|---|
| Voltage | Length | | | Burst length | Voltage | Length | | Burst length |
| (V) | (μsec) | Mode | Polarity | (μsec) | (V) | (μsec) | Mode | (μsec) |
| 950 | 120 | Burst | Unipolar | 20 | 100 | 23000 | Burst | 5 |

Following transfection, the cells were recovered from the cuvette into sterile TexMACS™ GMP medium (Miltenyi Biotec, #170-076-306) using a 18G sterile needle attached to a 1 mL syringe or similar tools. Cell count was performed using TC-20 automated cell counter (Bio-Rad). The cells were spun down at 300*g, 5 min, at room temperature. The supernatant was aspirated, and the cells were resuspended in sterile TexMACS™ GMP medium (Miltenyi Biotec, #170-076-306) supplemented with 100 ng/mL rhM-CSF (R&D systems, #AFL216), 20 ng/mL rhlL4 (R&D systems, #AFL204) and 20 ng/mL rhIL13 (R&D systems, #213-ILB/CF) at concentration 4*10\hat{ }6 cells/mL and plated at density 2*10\hat{ }6 cells/cm².

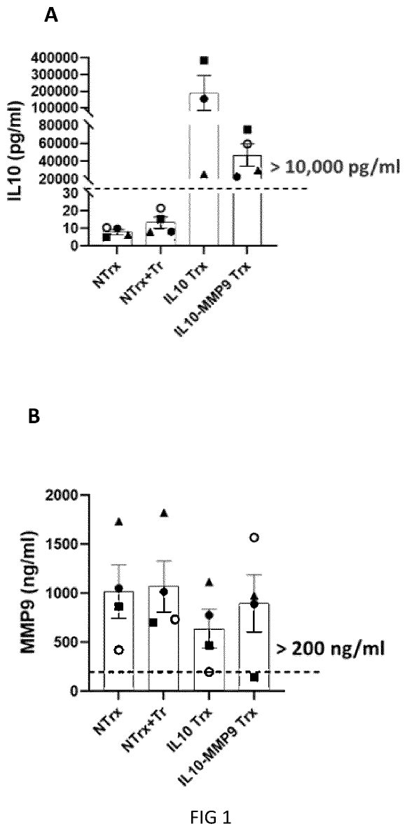

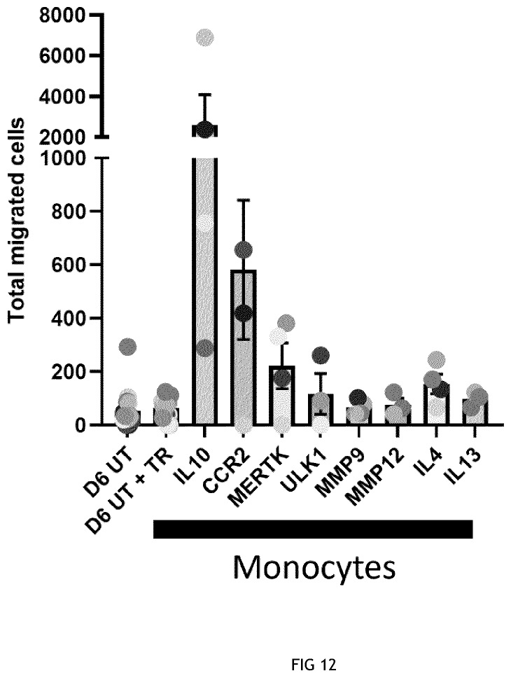

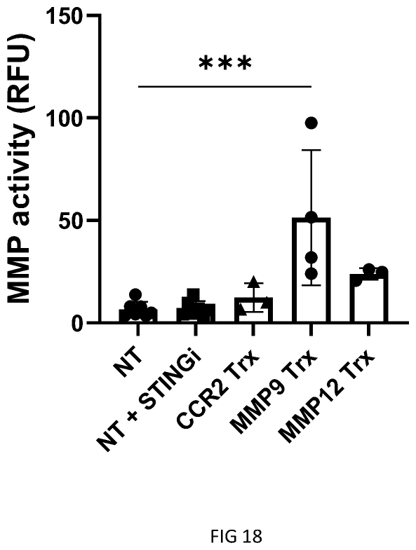

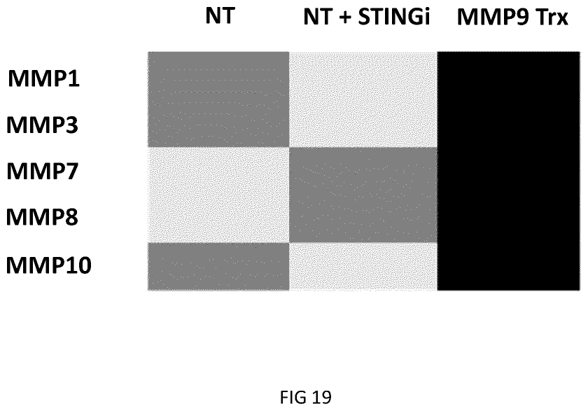

The macrophages used to generate the data shown in FIGS. 1-11 and 20-30 were transfected with RNA constructs. The macrophages used to generate the data shown in FIGS. 12-16, 18 and 19 were transfected by plasmid DNA constructs.

### Cryoresilience Treatment

To improve cryoresilience of the engineered macrophages following transfection, macrophages were incubated overnight in TexMACS serum-free medium (Miltenyi Biotec) containing 100 ng/mL M-CSF (BioTechne) and 20 ng/mL IL-4 and IL-13 (BioTechne) at a cell concentration of 4×10⁶ cells/mL and 2×10⁶/cm².

### Phagocytosis Assay

Briefly, macrophages were prepared for imaging analysis by plating them at a density of 150,000 cells/well in a 96-well clear bottom imaging plate (Grenier). Supernatant was removed and cells were stained for 30 mins with 100 ul PBS+NucBlue (ThermoFisher)+5 μg/ml Cellmask Deep red plasma membrane stain (Invitrogen) at 37 degrees Celsius, 5% CO2. Cells were washed three times with 100 μl PBS. 50 μl of PBS was added to cells for T0 analysis on the Opera Phenix High Content Screening System. 50 μl of 0.2 mg/ml pHrodo Red Zymosan beads (Life Technologies) were added to cells after T0. A series of images were acquired over a period of 96 minutes to monitor phagocytosis. Images were analysed with Columbus data imaging software and Tibco Spotfire data analysis system. Graphs plotted using GraphPad Prism 9.2.0.

The cells were prepared for flow cytometric analysis by resuspending them at a concentration of 2×106/ml in PBS+0.5 mM EDTA (Life Technologies). 50 μL of the cell suspension was dispensed into low adherence, round bottomed 96 well plates. 50 μL of resuspended pHrodo Beads (prepared as per manufacturer's instructions) were added to the test wells. Cells were incubated for 2 hours at 37° C. 5% CO2. At the end of the 1 hr incubation, the plate was spun at 300×g, 4° C., 5 min, supernatants eliminated and the pellets were resuspend with 100 μL of 1:100 FcR block PEA solution/well. Following incubation for 15 min at 4° C. in dark then ad antibodies (see Table 3) to appropriate test wells and incubate for 20 min at 4° C. Wash cells with PBS+0.5 mM EDTA and spin at 300 g for 5 min. Flip off the supernatants and resuspend cells in PBS+0.5 mM EDTA+1:1000 DRAQ7. Incubate for 5 min at 4 C. Wash as before, then resuspend in 100 ul of PBS+0.5 mM EDTA+0.1% human serum. Acquire 50 ul of cells on the Novocyte3000 or Novocyte Quanteon (Agilent). Flow cytometry analysis was conducted on NovoExpress software and the following gating strategy was utilised to identify actively phagocytosing macrophages: “Cell gate” to exclude debris, “singlet gate” to exclude cell doublets, “live gate” to exclude dead cells, “CD14+ gate” to identify iMACS and “phRodo+ve gate” to measure the percentage of phagocytosing macrophages.

### IL-10 Capture Assay

For each condition, 1×10⁶ cells were resuspended in 80 uL cold TexMACS and added with 20 uL IL-10 Catch Reagent, incubated for 5 min on ice. 10 mL warm TexMACS was then added to the payload transfected cells (test group) and 10 mL cold TexMACS was added to another tube of payload transfected cells as a negative control. Test group was incubated for 1.5 hr at 37° C. under continuous rotation. Negative control was kept on ice. After the incubation, cells were topped up with cold buffer to 15 mL and spun down at 4° C. Cells were then washed again with 10 mL cold buffer and spun down at 4° C. Cells were resuspended in 80 uL cold buffer, added with 20 uL detection antibody and 5 uL CD14 VioBlue and incubated on ice for 10 min. Cells were wash with 5 mL cold buffer and spun down at 4° C. Cells were then resuspended with 1 mL of 1:1000 Draq7 in cold buffer and 100 μL of the sample was transferred to a 96 w plate. Plate was spun down, resuspended with 100 uL cold buffer and acquired on flow cytometer.

### Flow Cytometry Labelling

Macrophages were resuspended at a concentration of 1×10⁶/ml in PBS+0.5 mM EDTA (Life Technologies)+FcR Block 1:100 (Miltenyi). Aliquot 100 ul of cells into low adherence, round bottomed 96 well plates. Incubate cells for 5 minutes, then add appropriate antibodies (see Table 2) to appropriate test wells and leave for 20 min at 4 C. Wash cells with PBS+0.5 mM EDTA and spin at 300 g for 5 min. Flip off the supernatants and resuspend cells in PBS+0.5 mM EDTA+1:1000 DRAQ7. Incubate for 5 min at 4 C. Wash as before, then resuspend in 100 ul of PBS+0.5 mM EDTA+0.1% human serum. Acquire 50 ul of cells on the Novocyte3000 or Novocyte Quanteon (Agilent).

TABLE 2
Antibodies used for flow cytometry labelling of hMDMs.

| | | | | ORDERING |
|---|---|---|---|---|
| ANTIGEN | FLUOROPHORE | CLONE | SUPPLIER | CODE |
| CD45 | VB | 5B1 | Miltenyi | 130-092-880 |
| | | | Biotec | |
| CD14 | VB/PE | TUK4 | Miltenyi | 130-091-242/ |
| | | | Biotec | 130-113-152 |
| CD206 | FITC | | Miltenyi | 130-095-131 |
| | | | Biotec | |
| 25F9 | APC | 25F9 | eBioscience | 50-0115-42 |
| CCR2 | PE | K036C2 | BioLegend | 357206 |
| CD163 | FITC | | Miltenyi | BO-097-626 |
| | | | Biotec | |
| CD169 | APC | 7-239 | BioLegend | 346008 |
| CD86 | PE | BU63 | BioLegend | 374206 |
| MHC II | FITC | TU39 | BioLegend | 361705 |

### MSD V-Plex Cytokine Dosage

Cytokines in cell culture supernatants cytokines were analysed using a V-PLEX Human Biomarker 10-Plex kit on a MESO Quickplex SQ 120 according to the manufacturers' instructions (Meso Scale Discovery. 10 uL of supernatants were tested. Results are in pg/mL. Values are adjusted taking into consideration the dilution at the time of testing. All data shown represent the secretion in a 24 h period. Data reported are net concentration, as calculated by subtracting the amount of the given cytokine in culture medium alone (TexMACS) to the amount of cytokine detected in the cell culture supernatants.

### PBMCs Attraction/Migration Assay

Buffy coat donations were purchased from SNBTS under sample governance 20^{˜}17. Peripheral blood mononuclear cells were isolated from the buffy coats using standard methods and frozen at a density of 50×10⁶/mL in CryoStorCS10 (STEMCELL) and stored until required. To set up the migration assay, PBMCs were thawed and resuspended at 4.6×10⁶ in TexMACS (Miltenyi). 75 ul of cells in TexMACS were added to the top chamber of a 5 micron 96 well transwell (Corning). 150 ul of frozen and thawed conditioned medium was added to the bottom chamber. The transwell plate was placed in an incubator (37 C, 5% C02). After 3 hours, the top chamber was removed and the migrated cells in the bottom chamber were stained with CD45-PerCP, CD14-VioBlue, CD15-Pevio770, CD16-BV605, CD56-PE, CD3-FITC and CD19-APC using a standard flow cytometry staining procedure and acquired with a Novocyte3000 or Novocyte Quanteon (Agilent).

### Polarisation Assay—Non-Polarised Macrophages

Mature day 5 macrophages were seeded on a 96-well plate (Corning) at a density of 2×10⁶/cm² in TexMACS+100 ng/ml of MCSF (R&D). 50 ng/ml of IL-10 (R&D) was added to control wells (M2 polarisation medium). After cells had attached (5 hours), the medium was removed and replaced with conditioned medium. Cells were incubated for 18 hours with conditioned medium in an incubator (37 C, 5% C02), then stained for CD14-VioBlue, CD45-PerCP, CD206-BV711, 25F9-eF660, HLA-DR-PeCy7, CD86-PE, CD163-FITC, using a standard flow cytometry staining protocol. DRAQ7 was used to stain dead cells. Cells were then acquired with a Novocyte3000 or Novocyte Quanteon (Agilent).

### MMP Activity Assay

MMP activity was confirmed via successful cleavage of a standard MMP peptide. The standard MMP peptides are flanked with a quencher and fluorescent signal, which when intact, do not fluoresce. Cleaved peptides no longer quench the fluorescent signal, therefore resulting in an emission of fluorescence which is measured as relative fluorescent units (RFU). The assay was carried out in accordance with the manufacturer's instructions (https://www.abcam.com/ps/products/112/ab112146/documents/ab112146%20MMP %20Activity %20Assay %20Kit %20Fluorometric %20-%2OGreen %20v4b %20(website).pdf—ab112146 MMP Activity Assay Kit Fluorometric—Green v4b) 25 ul of cell culture supernatants were tested. All data shown represent the activity of MMPs secreted over a 24-hour period. Results are plotted as RFU minus background fluorescence of culture medium alone (TexMACS).

### Statistics

Every reported dot is a distinct donor. At least 3 donors were analysed in each condition unless otherwise specified. Where appropriate, data are shown as mean±SD. Where appropriate, two tail t-test for paired data was carried out.

### Results

### Example 1: Efficient Transfection of IL-10 and IL-10+MMP9 in hMDMs

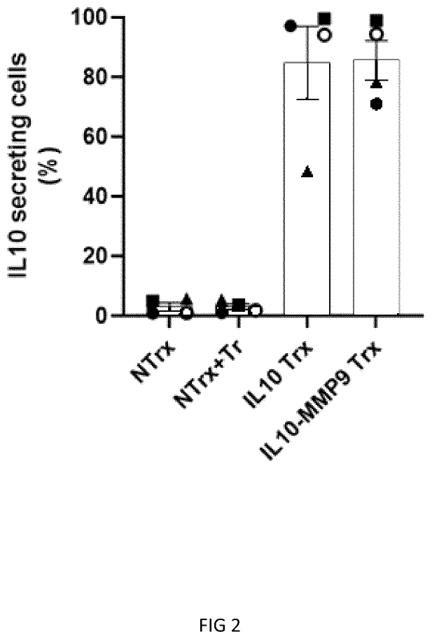

To generate transfected (Trx) human monocyte-derived macrophages (hMDMs) for cell therapy, it is necessary to obtain significant increase in the level of expression and secretion of the desired payloads. We set minimal thresholds for our payloads of choice, IL-10 and MMP9, based on in-house and published results. Levels of secreted protein are measured in the cell culture supernatants 24 h post-transfection by ELISA. Interestingly, both IL-10 and IL1-O+MMP9 (bicistronic vector) transfection resulted in significant increase in IL-10 secretion (FIG. 1). Increase in IL-10 secretion in engineered macrophages has also been confirmed with a flow cytometry based IL-10 capture assay, which monitors the secretion of IL-10 in macrophages in a period of 2 hours (FIG. 2). However, MMP9 secretion is not dramatically increased: IL-10 only transfection results in a slight decrease in the secretion of MMP9, which is at least partially rescued by the co-transfection of IL-10 and MMP9 (FIG. 1), suggesting a sufficient transfection efficiency of MMP9. In all the experiments presented herein we always analyse non-transfected (NTrx), NTx+recovery treatment (IL4+IL13), IL-10 only Trx and IL-10+MMP9 Trx. All Trx cells receive the recovery treatment. IL-10 and MMP9 are co-transfected using a bi-cistronic vector, linked by a p2A linker sequence.

### Example 2: IL-10 and IL-10+MMP9 Transfected hMDMs Show Preservation of Macrophage Identity Markers

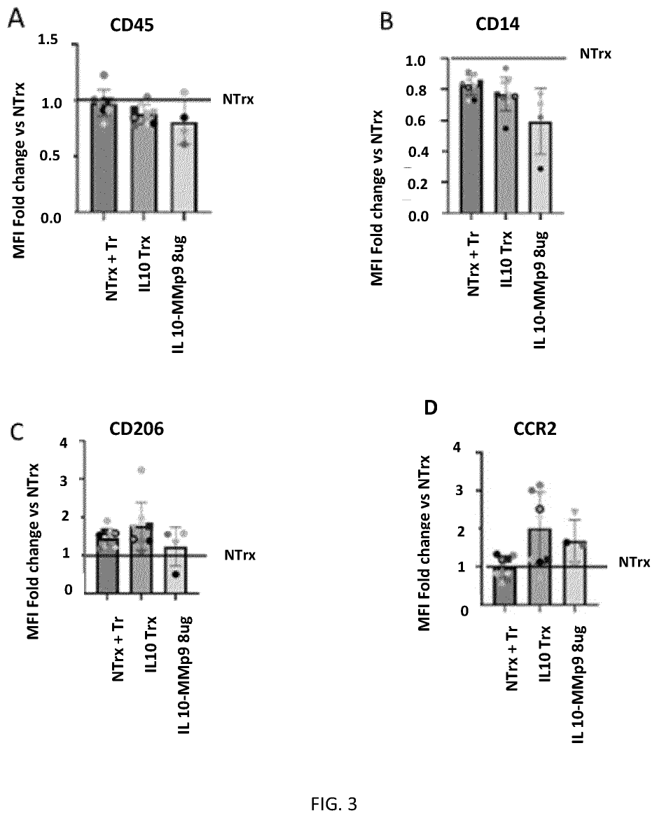

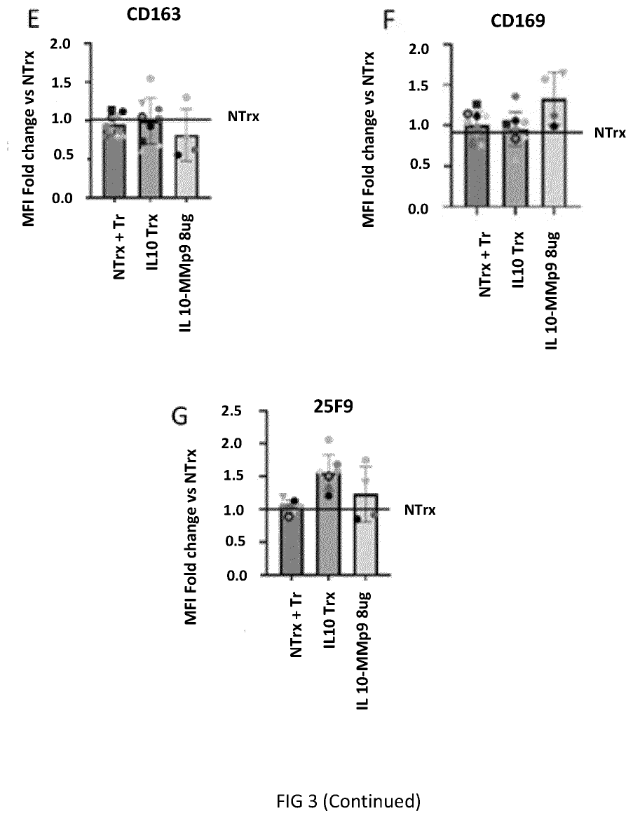

Pivotal to obtain safe and effective macrophage cell therapies is the maintenance of macrophage cell surface identity markers. This shows that the genetic engineering procedure does not fundamentally change the identity of the cells. Pan-leukocyte marker CD45 is preserved in all cell types. In IL-10+MMP9 there is a slight decrease in the intensity of expression of the myeloid marker CD14 (MFI Fold change), but the percentage of positive cells is unchanged, therefore we are satisfied that the cells maintain their myeloid identity. The mature macrophage marker 25F9 is slightly increased in the engineered cells, which is a positive sign of strong macrophage identity. All other markers analysed (CD206, CD163, CCR2, CD169) are unchanged or slightly increased in engineered vs. non-engineered cells. These data support the idea that our engineering method is safe and does not perturb the identity of the cells. Data is depicted in FIG. 3.

### Example 3: IL-10 and IL-10+MMP9 Transfected hMDMs have a Marked Anti-Inflammatory Profile

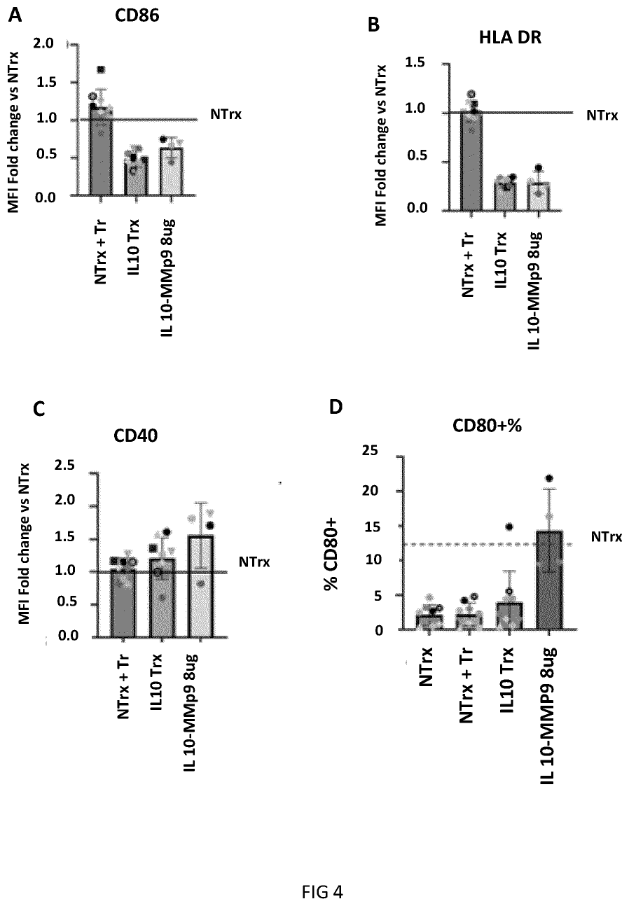

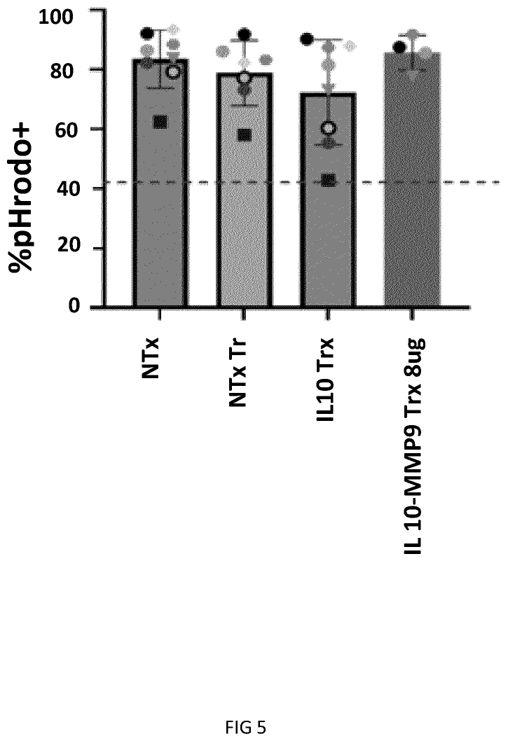

To treat acute and chronic inflammatory conditions, e.g. related to organ damage, it is important to obtain a highly anti-inflammatory macrophage. Surprisingly, not only the transfected macrophages retain all the identity markers (FIG. 3), but they also show a reduction in several pro-inflammatory markers, such as CD86 and HLA-DR (FIG. 4). This underlines an autocrine-paracrine effect of the engineered IL-10 in inducing a strong anti-inflammatory phenotype in engineered macrophages. The slight increase in CD80 level observed with IL-10+MMP9 is unlikely to be significant in terms of biological effect, as it is within 10% of the desired levels (red dotted line).

### Example 4: IL-10 and IL-10+MMP9 Transfected hMDMs have Excellent Phagocytic Capacity

Another key aspect of an efficacious macrophage cell therapy in the context of acute and chronic inflammatory conditions e.g. related to organ damage is its ability to phagocytose efficiently. Herein, we report that both engineered and non-engineered macrophages phagocytose efficiently, and above the minimal desired levels (dotted line) (FIG. 5).

### Example 5: IL-10 and IL-10+MMP9 Transfected hMDMs Polarize Naïve Macrophages to a Pro-Restorative Phenotype

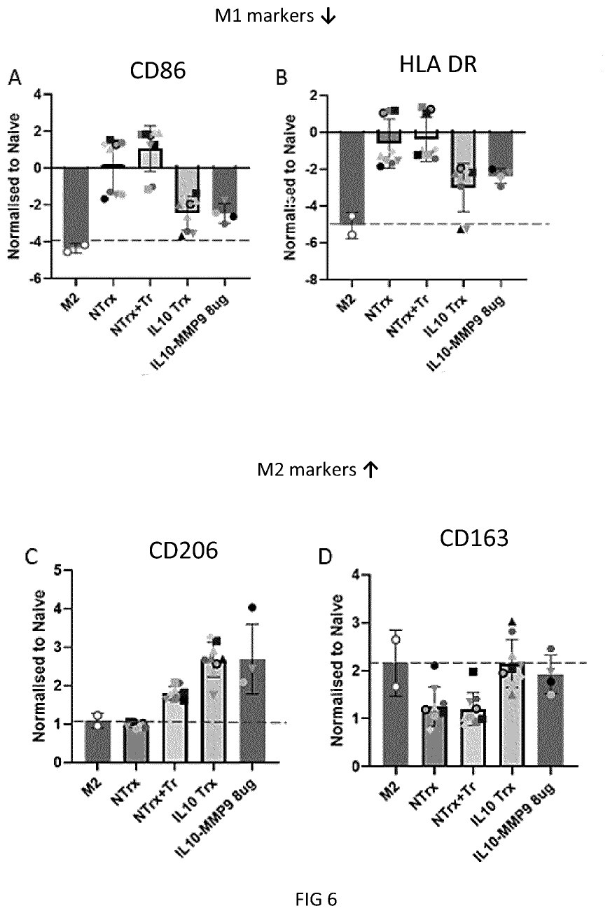

During inflammatory organ damage, local macrophages need to acquire a pro-restorative phenotype to support fibrosis remodelling and/or tissue regeneration. In this assay we assess the ability of cell culture supernatants from NTrx, NTrx+Treatment, IL-10 Trx and IL-10+MMP9 Trx macrophages 24 h post-transfection to polarize macrophages from an unrelated donor. M2 macrophages polarized from the same donor using recombinant IL-10 are used as positive control (red dotted line). The desired outcome is for M1-type markers CD86 and HLA-DR to decrease and M2-type markers CD206 and CD163 to be similar or increase in macrophages treated with supernatants from engineered vs. non-engineered hMDMs. Supernatants from both IL-10 Trx and IL-10+MMP9 Trx hMDMs are effective and promotes conversion of unrelated donor macrophages to a pro-restorative phenotype (decrease in CD86 and HLA-DR, increase in CD206 and similar levels of CD163) (FIG. 6).

### Example 6: IL-10+MMP9 Transfected hMDMs have Better Monocyte Recruitment Capabilities Vs. IL-10 Transfected hMDMs

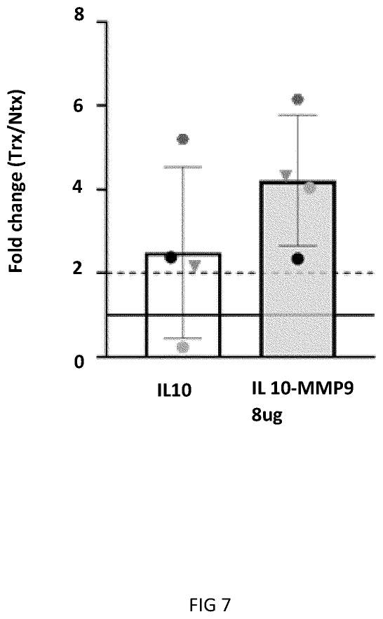

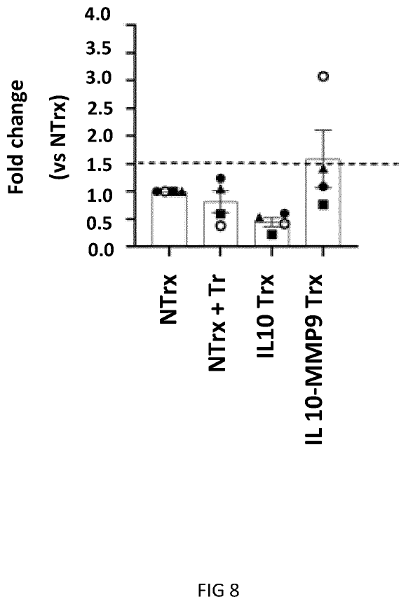

An important function of macrophages is to recruit new monocytes on site, so they can then be patterned towards a pro-restorative phenotype. Evidence of the ability of IL-10 Trx and IL-10+MMP9 Trx hMDMs to induce such phenotype in non-polarised macrophages was provided in FIG. 6. Data shown in FIG. 7 support the notion that, surprisingly, only supernatants from IL-10+MMP9 Trx hMDMs induce significant monocyte migration when tested in a PBMC migration assay.

### Example 7: IL-10+MMP9 Transfected Macrophages are Superior in Terms of Metalloproteases Activity Vs. IL-10 Transfected Macrophages

The last key feature in a macrophage cell therapy aimed at inducing tissue remodelling is their ability to digest extracellular matrix (ECM) components. This assay measures the ability of the overall pool of MMPs in cell culture supernatants to digest ECM components via fluorescence probes. Surprisingly, IL-10 Trx greatly reduced (−50%) the overall MMP activity measured in the supernatant as compared to NTrx hMDMs. It is remarkable that the co-transfection of MMP9 only is sufficient to restore such ability in the supernatants, and to have it going even higher than in NTrx hMDMs. hMDMs co-expressing IL-10 and MMP9 have 1.5× the MMP activity than NTrx hMDMs, suggesting that MMP9 and IL-10 synergize to increase the MMP activity of hMDMs. This could underline the ability of MMP9 transfection to increase the activation of other MMPs, too. Data is depicted in FIG. 8.

### Example 8: IL-10 and IL-10+MMP9 Transfected hMDMs Localize in the Liver 24 h and 72 h Post-Injection and are Rapidly Cleared from the Lungs in Models of Chronic Liver Disease

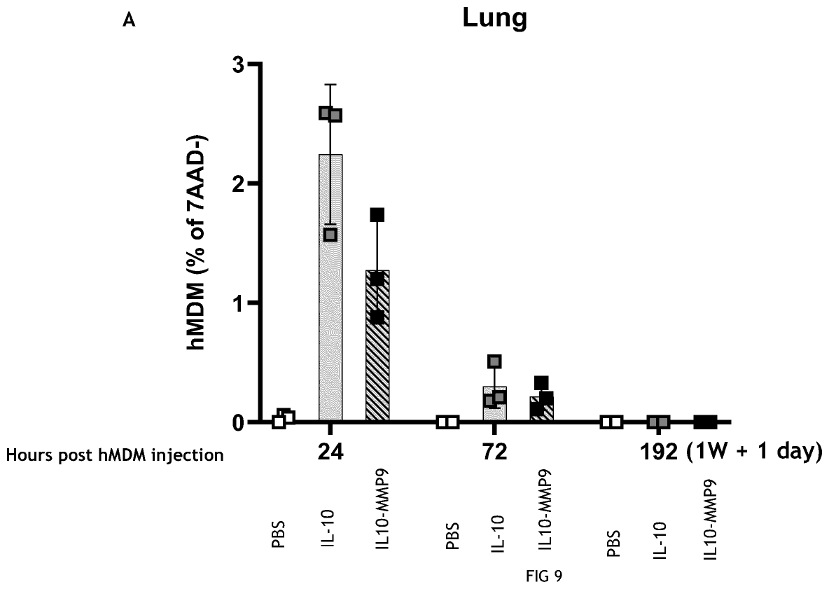

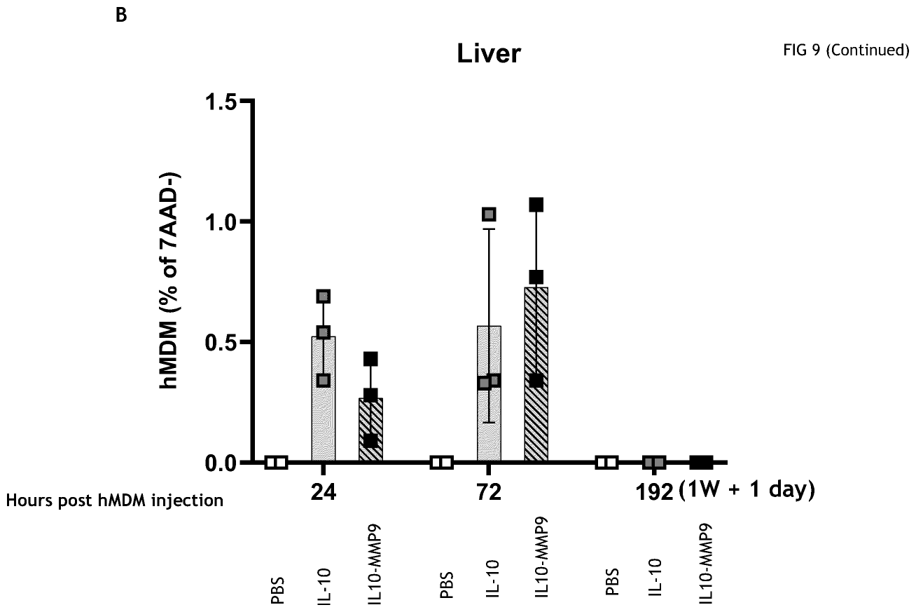

In order for a macrophage cell therapy to be efficacious, it needs to locate at the site of damage post-injection. In this experiment, chronic liver disease is induced in immunodeficient mice (NSG strain) by injecting a toxin (CCl₄). After four weeks of fibrosis induction hMDMs were injected via tail vein, livers retrieved at distinct time points, and digested using enzymes to retrieve the non-parenchymal fraction. Results show that both IL-10 Trx and IL-10+MMP9 Trx hMDMs locate first to the lung and liver but persist in liver only at 72 h. By one week post-injection the genetic engineered cells are cleared, as expected (FIG. 9). Therefore, they show a pharmacokinetic and distribution compatible with efficacy and safety.

### Example 9: IL-10 and IL-10+MMP9 Transfected hMDMs Maintain Expression of the Payloads 24 h Post-Injection in Models of Chronic Liver Disease

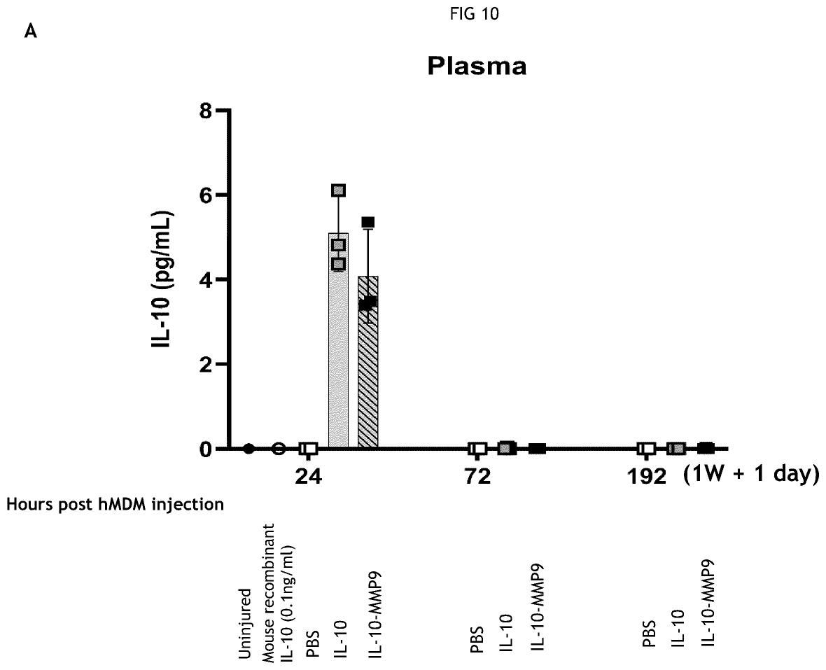

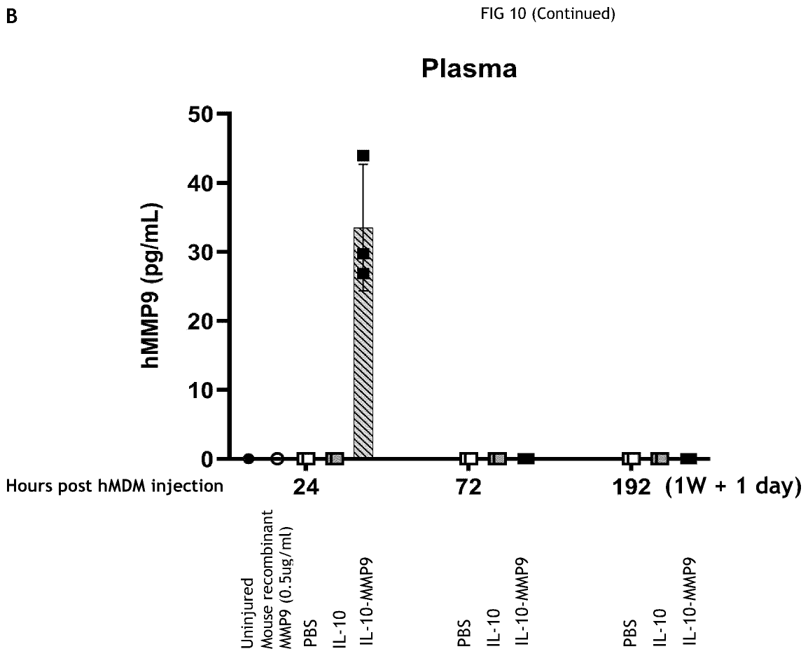

In the same experiment outlined above circulating human IL-10 and MMP9 were measured in the plasma of mice at distinct time points following injection of the cell therapy. Interestingly, despite macrophages concentrating in liver and lung, both IL-10 and MMP9 are detected systemically (circulating in blood) in the expected groups of mice (FIG. 10). Appropriate controls were carried out to ensure that the human proteins were detected reliably, with no cross-reactivity with their mouse counterparts.

### Example 10: IL-10 and IL-10+MMP9 have a Good Safety Profile in Models of Chronic Liver Disease

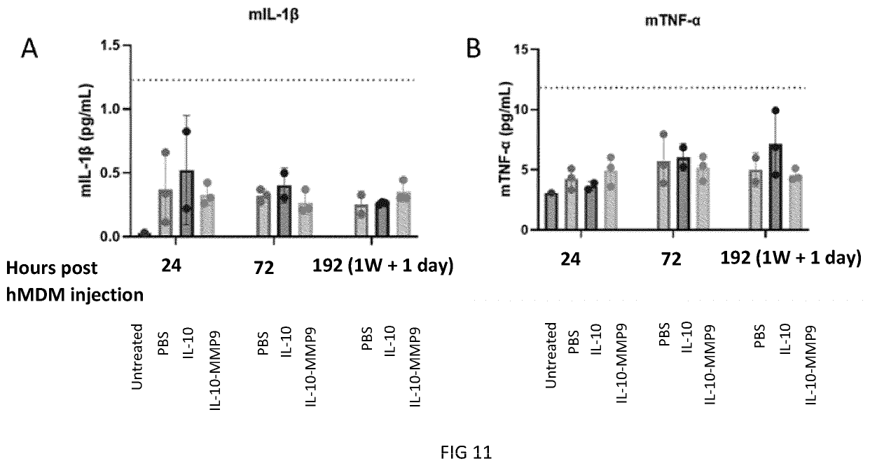

Finally, to be successful in a clinical setting, a cell therapy needs to be safe, both at the time of injection and at various later time points. Particularly, it is paramount for the cell therapy to have no off-target effect in non-damaged organs, and it is cleared within a safe time frame. Data support safety of both IL-10 Trx and IL-10+MMP9 Trx hMDMs. In fact, no embolism was detected at the time of injection, and no systemic inflammation was detected systemically in mice with chronic liver disease at various time point post-injection (Table 3 and FIG. 11). These data, coupled with the rapid clearance observed in FIG. 10, negate the possibility of these cells to be tumorigenic or to have any long-term toxicity.

TABLE 3
summary of desired safety features and results to satisfy safety requirements.

| Task | Success Criteria | IL10 Trx | IL10-MMP9 Trx |
|---|---|---|---|
| Safety: Increase in circulating miL-1β and | <=3 fold Tx-hMDM vs. PBS | IL1b: 1.4 FCh (24 hr) | IL1b: 0.86 FCh (24 hr) |
| mTNFα | | TNFa: 0.86 FCh | TNFa: 1.2 FCh |
| Safety: pulmonary embolism within 30 min | No embolism | No embolism | No embolism |
| from injection (max 5 × 10\hat{ }6 i.v. in NSG). | | | |

### Example 11: Engineered Macrophages Overexpressing IL-10 Specifically Recruits Monocytes

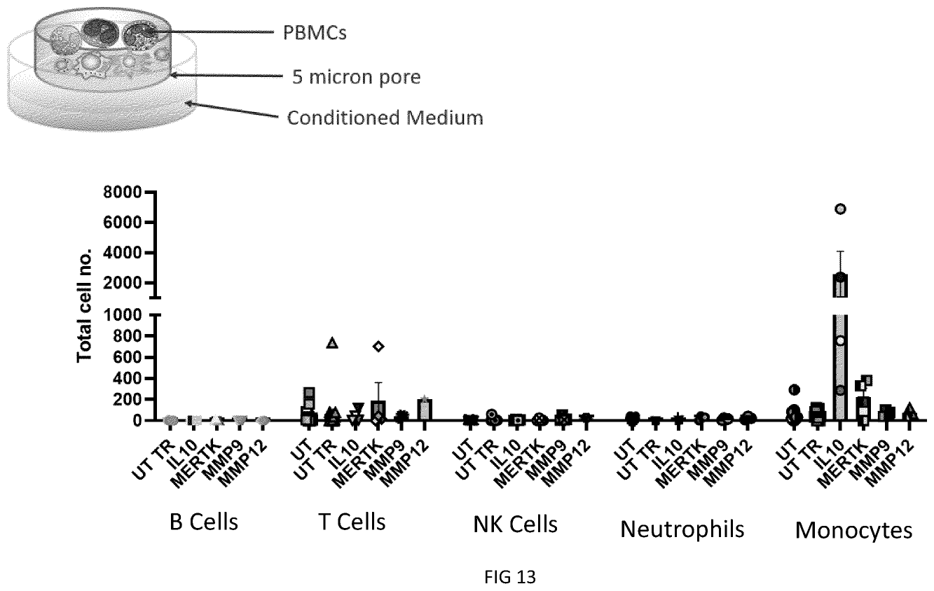

Migration of PBMCs was measured as described herein in response to conditions medium from hMDMs treated as described in FIG. 12. “D6 UT” cells were taken after 6 days of culture as described herein, and not transfected and not treated with IL-4 or IL-13 (recovery treatment as described herein). “D6 UT+TR” cells were taken after 6 days of culture as described herein, not transfected, but were treated with IL-4/IL-13 (recovery treatment as described herein). The remaining treatment groups were transfected with the genes as indicated. Transfection with IL-10 alone induced the greatest monocyte recruitment, with transfection with CCR2 the only other treatment producing macrophages capable of significant monocyte recruitment. FIG. 12 only shows the recruitment of monocytes as identified by flow cytometry. FIG. 13 shows the recruitment among other cells types by macrophages treated with a subset of the conditions shown in FIG. 12 (UT and UT TR correspond to the D6 UT and D6 UT TR cells described in FIG. 12) in as identified using flow cytometry (B cells, T cells, NK cells, Neutrophils and Monocytes). FIG. 13 demonstrates that IL-10 transfected cells specifically recruit monocytes, without recruiting other cell types. Conditioned medium from untransfected macrophages, and those overexpressing MMP9, had no effect on monocyte recruitment.

### Example 12: IL-10 Overexpressing Engineered Macrophages Convert Both Unpolarized and Pro-Inflammatory Macrophages to a Pro-Restorative Phenotype

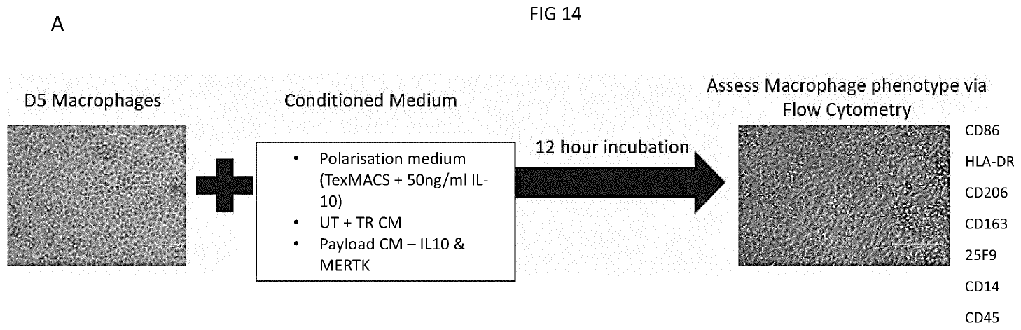

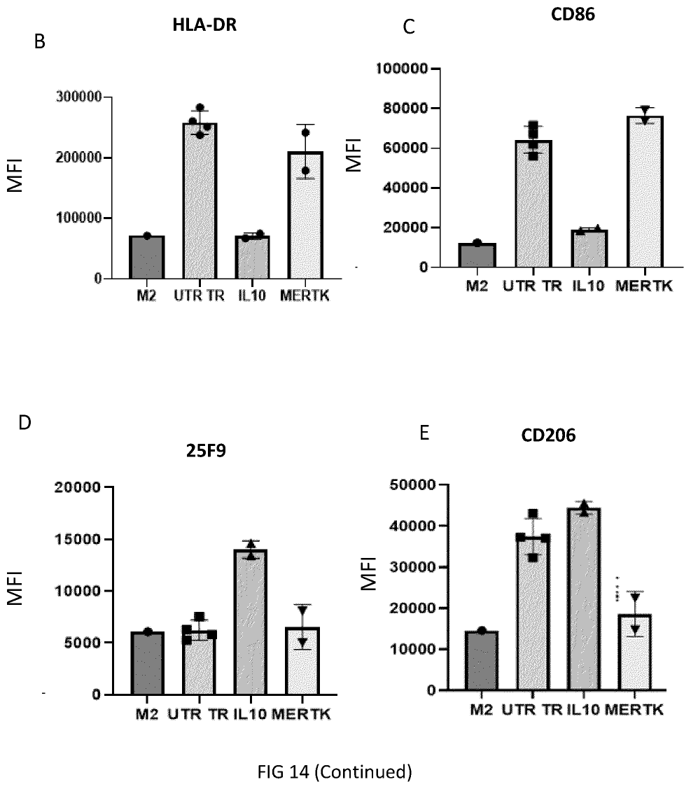

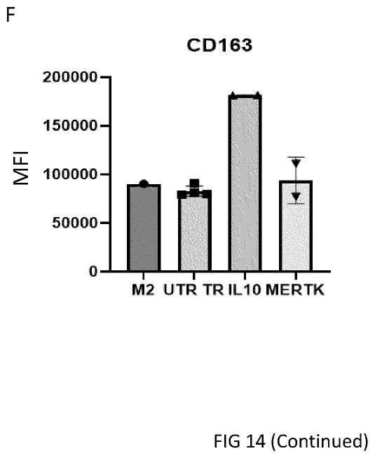

To test the ability of IL-10 over-expressing macrophages to convert monocyte derived macrophages to pro-restorative macrophages, the inventors tested the effect of CM from IL-10 overexpressing macrophages on the phenotype of on M0 and M1 macrophages. When M0 macrophages (derived from monocytes that were incubated for 5 days in the presence of 100 ng/ml recombinant human macrophage colony-stimulating factor, rhM-CSF, essentially as described in WO 2021/240162) were incubated with CM from IL-10 overexpressing cells, they displayed a marker profile associated with pro-restorative M2 macrophages (i.e. downregulation of HLA-DR & CD86 and upregulation of 25F9, CD206 and CD163). The effect was equivalent to incubating the M0 macrophages with media containing M2 polarisation medium (TexsMACS+50 ng/ml IL-10) (FIG. 14). The surface markers were assessed using flow cytometry and measuring Mean Fluorescence Intensity (MFI).

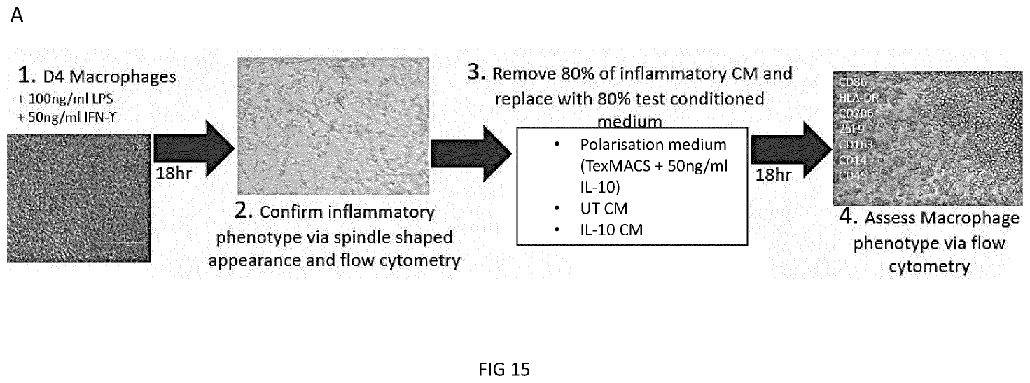

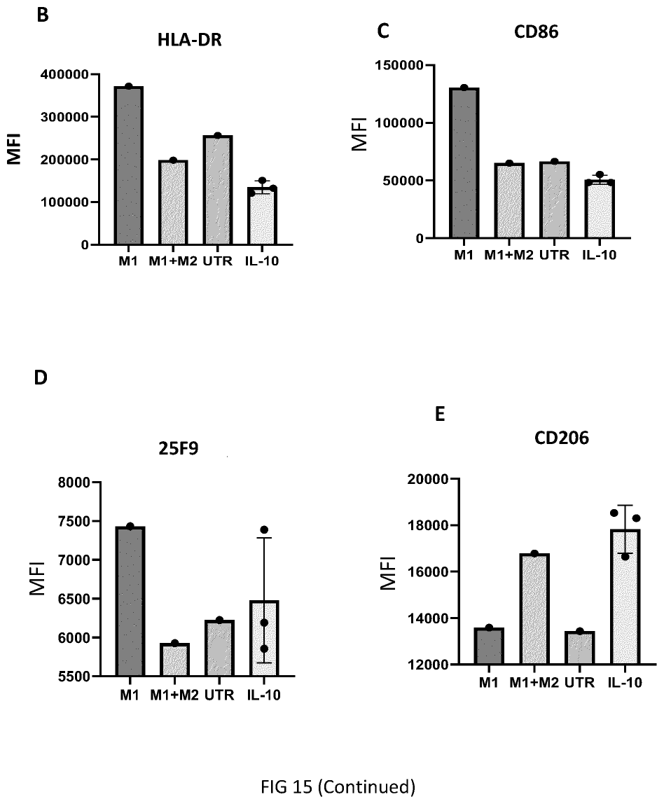

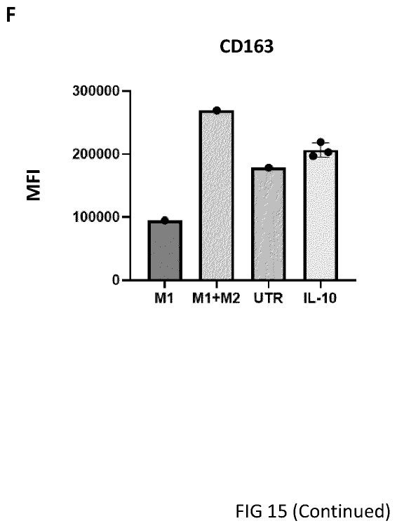

Similarly, the CM in which IL-10 overexpressing macrophages were incubated was able to “rescue” M1 macrophages and convert their phenotype to a pro-restorative M2 phenotype. To test that the CM was incubated with macrophages that were pre-polarised to an M1 phenotype using 100 ng/ml LPS+50 ng/ml IFN-γ. This rescue ability was similar to that of using an M2 polarisation medium (FIG. 15).

### Example 13: Macrophages Engineered to Overexpress IL-10 have an Anti-Inflammatory Secretome Profile

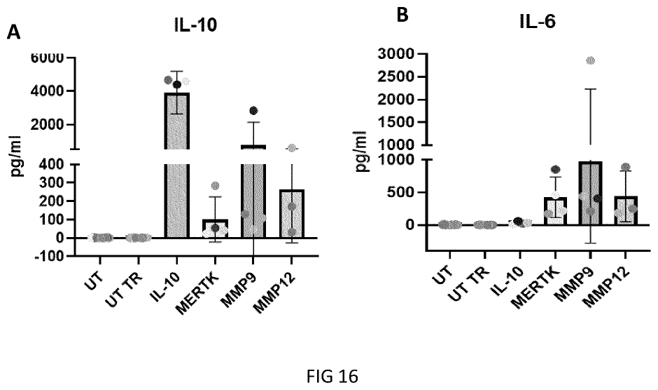

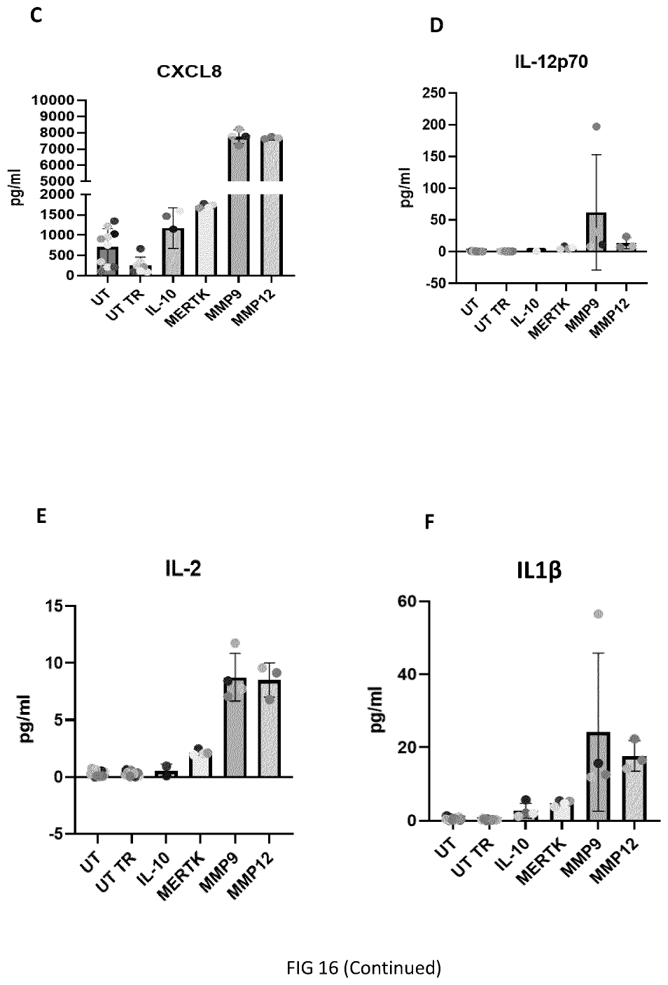

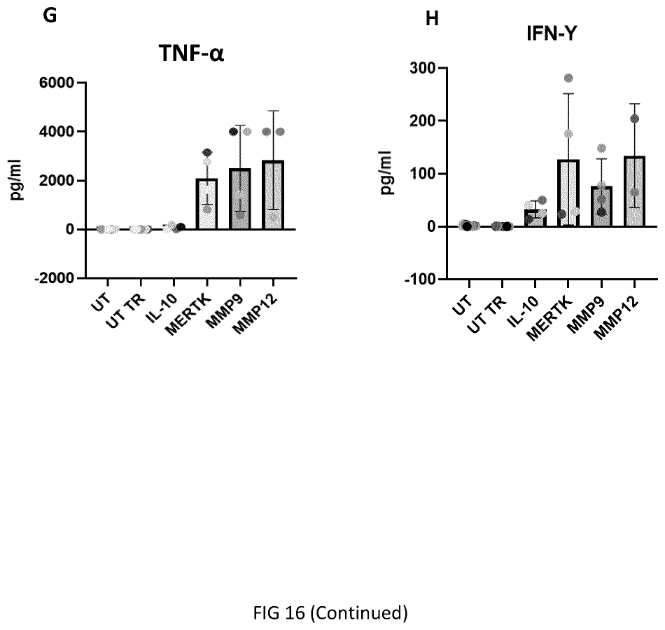

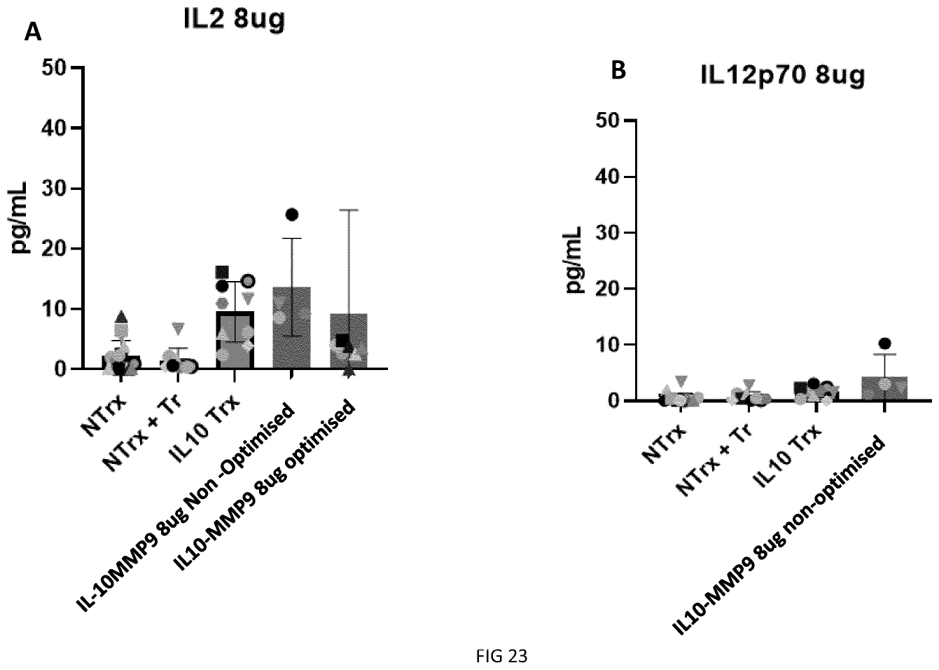

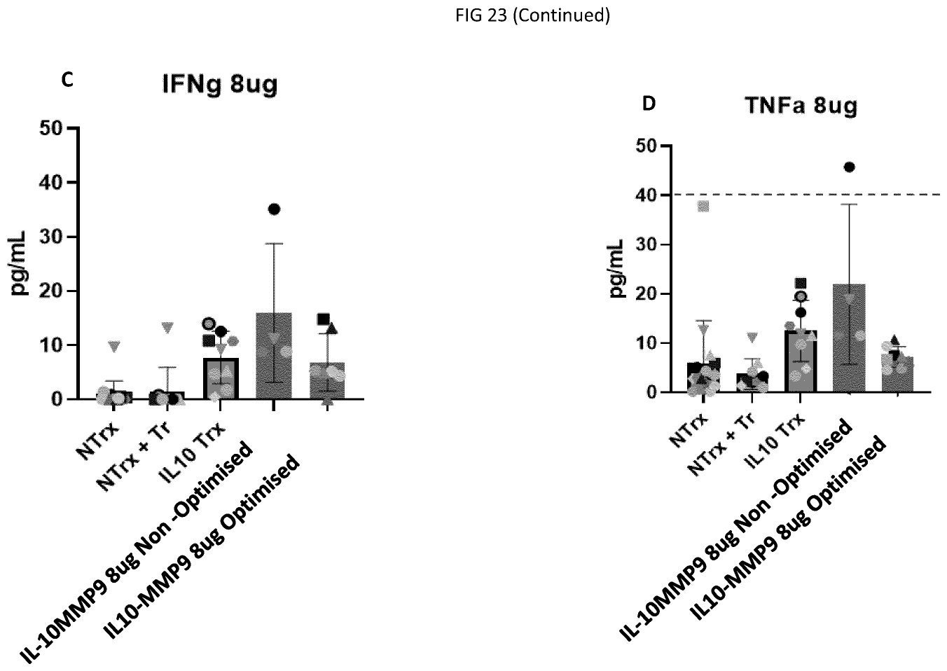

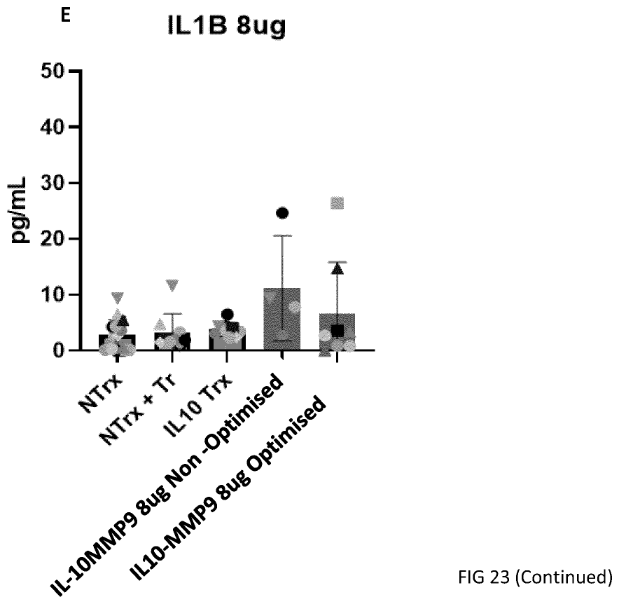

The secretion level of pro-inflammatory cytokines was measured in macrophages over-expressing IL-10 alone, macrophages over-expressing MMP9 alone, macrophages over expressing IL-10 and MMP9, and non-transfected non-polarised macrophages. Macrophages over-expressing IL-10 demonstrated an anti-inflammatory secretome profile. In particular, no secretion of pro-inflammatory factors such as TNF-α and IFN-γ was observed when macrophages over-expressed IL-10 (FIG. 16). Surprisingly, although macrophages over-expressing MMP9 alone showed an increased secretion of pro-inflammatory cytokines such as IL2, IL12p70, IFNg, TNFa and IL1β (FIG. 16), macrophages over-expressing MMP9 and IL-10 show a similar secretion level as that of macrophages over-expressing IL-10 alone (FIG. 23). Similarly, the effect of macrophages on systemic inflammation was evaluated in-vivo. Briefly, NSG mice were subjected to 4-5 weeks of CCl₄ intoxication. On SD 23, mice were randomized to receive macrophages over-expressing IL-10 alone (IL-10 Trx hMDM) or macrophages over-expressing IL-10 and MMP9 (IL-10-MMP Trx hMDM). Mice injected with Phosphate-buffered saline (PBS) were used as vehicle control. Mouse IL1β and TNFα levels were measured in liver homogenate by MSD assay. Surprisingly, despite the pro-inflammatory secretome profile of macrophages expressing MMP9 alone as measured in-vitro, it was found that both macrophages over-expressing IL-10 alone or IL-10 and MMP9 did not induce an increase in inflammatory cytokines as compared to vehicle control (FIG. 11).

### Example 14: Engineered Macrophages can be Delivered to and Persist in the Liver

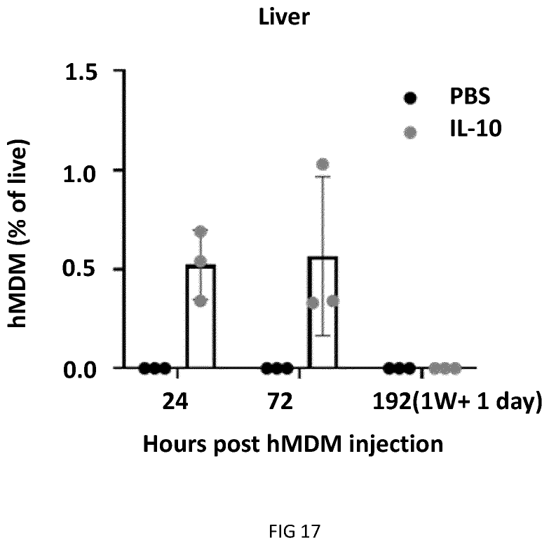

In addition, IL-10 overexpressing human macrophages which were intravenously injected into mice with modelled liver fibrosis localised to the liver and persisted there for at least 72 hours post administration, suggesting that they can be delivered to the therapeutic area in patients suffering from liver fibrosis (FIG. 17).

### Example 15: Engineered Macrophages have MMP and Scar Remodeling Activity

In order to rescue the ability of the IL-10 over-expressing macrophages to induce scar-remodelling, the inventors considered introducing a Matrix Metalloprotease (MMP), as MMPs are known to have an important role in degrading scar tissue in inflamed liver (for example, Campana et al, Nature Reviews Molecular Cell Biology volume 22, pages608-624 (2021)). As macrophages over-expressing either MMP9 or MMP12 demonstrated an ability to maintain some phagocytic ability, the inventors tested the scar-remodelling ability of macrophages expressing either MMP. By measuring total MMP activity using a FRET-based fluorophore method, it was observed that both MMP9 and MMP12 induced an increase in total MMP activity, with MMP9 inducing a higher increase in total activity (FIG. 1).

### Example 16—Macrophages Engineered to Overexpress Both MMP9 and IL-10 Recruit Monocytes in Vitro and in Vivo

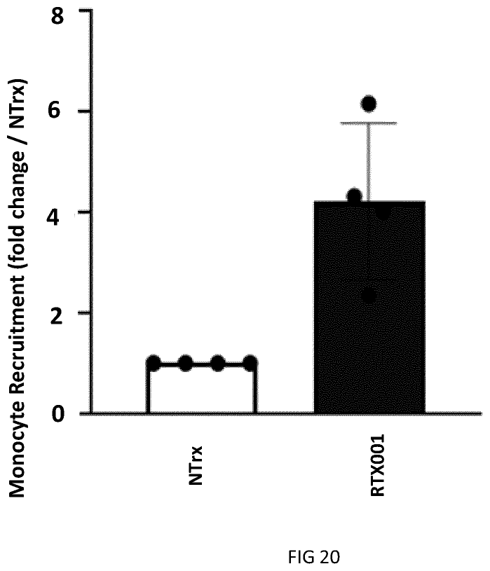

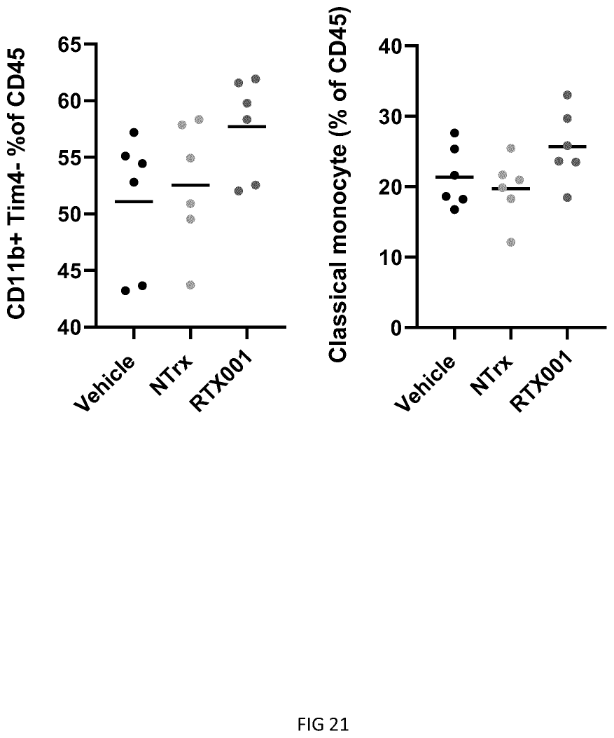

RTX001 macrophages were produced by transfecting hMDMs with a single bi-cistronic mRNA comprising sequences encoding both MMP9 and IL-10. FIG. 20 shows results from a PBMC migration assay was conducted as described in the Materials and Methods, comparing NTRx cells to RTX001 macrophages in vitro. The ability of macrophages over-expressing both MMP9 and IL10 to recruit monocytes has been also confirmed in-vivo in a mouse model of liver fibrosis (liver injury induced by 4-5 weeks of CCl4 intoxication) at 24 h post-administration. The results of this assay are shown in FIG. 21. NSG mice were subjected to 4-5 weeks of CCl4 intoxication. RTX001 and NTrx cells were administered i.v. on SD 23 and readouts were collected 24 hr post cell administration. Mice injected with Phosphate-buffered saline (PBS) were used as vehicle control. Flow cytometry enumeration of myeloid cell (defined as CD45+, SiglecF−, Ly6G−, Tim4−, CD11b+) and classical monocytes (classical monocytes defined as CD45+, SiglecF−, Ly6G−, Tim4−, CD11b+, CD64−, Ly6Chi) recruitment as percentage of mouse CD45 leukocytes was carried out. Migration in vivo was defined as cell recruitment to liver vs vehicle control. As can be seen in FIG. 21, mice receiving cells over-expressing IL-10 and MMP9 (RTX001) showed an increase in the percentage of monocytes (right panel) and myeloid cells (left panel) recruited out of total leukocytes when compared to mice treated with non-transfected cells (NTRx) or PBS.

### Example 17—Engineered Macrophages Convert Cells in the Liver to a Pro-Restorative Phenotype In Vivo

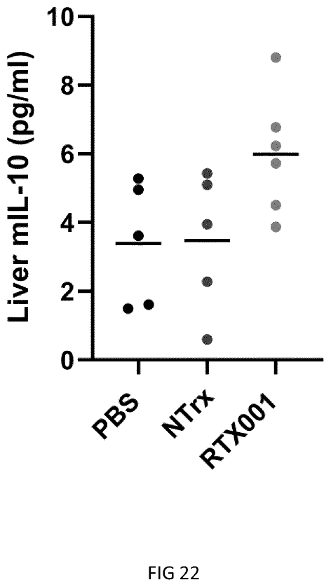

NSG mice were subjected to 4-5 weeks of CCl4 intoxication. RTX001 and NTrx cells were administered i.v. on SD 23 and readouts were collected 24 hr post cell administration. Mice injected with Phosphate-buffered saline (PBS) were used as vehicle control. Mouse cytokines, including IL-10 levels in liver homogenate were assessed by MSD assay. As can be seen in FIG. 22 administration of human cells over-expressing IL-10 and MMP9 (RTX001) was able to induce an increase in mouse IL-10 (as measured in the liver homogenate). This indicates that the human IL-10 induced an anti-inflammatory phenotype, which suggests conversion of mouse cells to a pro-restorative phenotype in view of IL-10's well-known polarising characteristics (of note, non-transfected non-polarised cells did not induce any increase in the level of mouse IL-10).

### Example 18—RTX001 Macrophages Reduce a-SMA Expression In Vitro and In Vivo

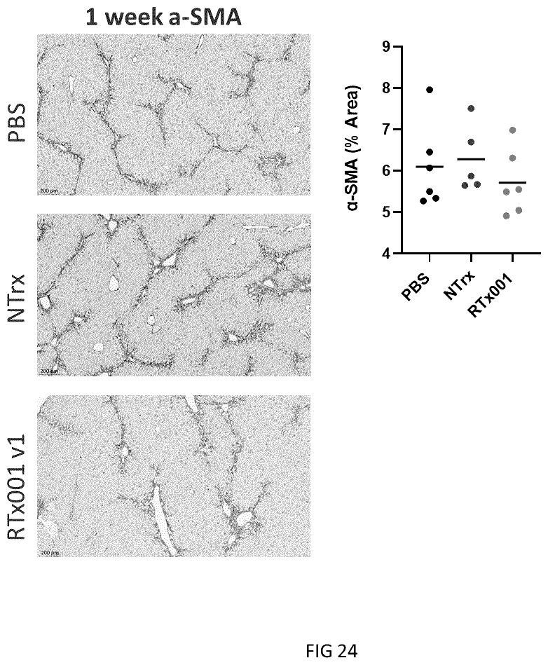

The beneficial effect of macrophages over-expressing IL-10 and MMP9 has been demonstrated in-vivo in a mouse model of liver fibrosis (mice intoxicated for 4-5 weeks with CCl4) at 1 week post-administration. When the macrophages were intra-venously injected to the mice, a reduction in activation of scar generating cells (i.e. activated hepatic stellate cells, HSCs) has been observed within the scar tissue as compared to non-transfected (NTrx) macrophages and PBS controls (visualized by staining for a-SMA, which is an activation marker of HSCs, which was quantified in the result in the right panel) (FIG. 24).

To further demonstrate the effect of macrophages expressing IL-10 and MMP9 (RTXOO1) on liver fibrosis, an in-vitro system was developed using the LX-2 cell line (Sigma Aldrich, SCC064). The LX-2 line is a human hepatic stellate cell line that has been extensively characterized and shown to retain key features of hepatic stellate cells and thus a suitable model of human hepatic fibrosis. In the system, the LX-2 cells were treated with 50 ng/ml TGF-β in DMEM media for 24 h to activate the cells, which occurs during liver fibrosis. The cells were then incubated for an additional 24 h with DMEM conditional media (CM) in which the following macrophages were grown for about 18 h:
1. Untreated/Non-transfected/UT human macrophages (Non-Trx CM).
2. Human macrophages expressing a construct encoding IL-10 and MMP9 (RTX001 CM).
3. Human macrophages expressing a construct encoding IL-10 alone (Control CM).

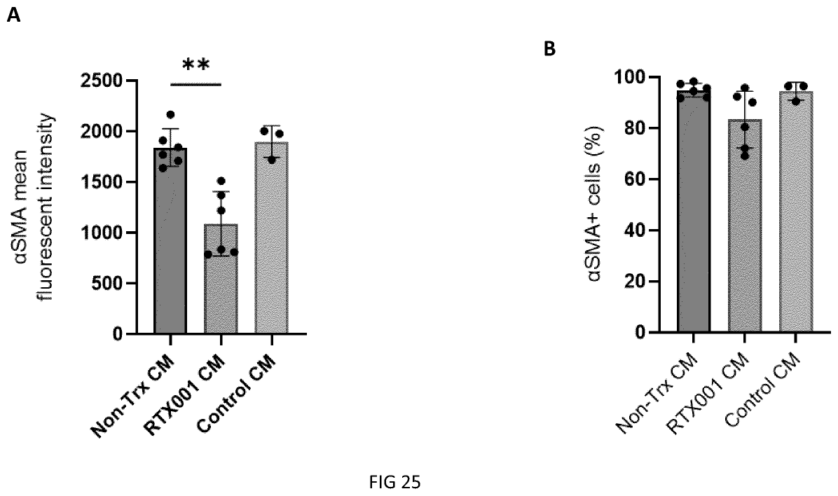

For each of these groups, conditioned media from macrophages of 6 different donors were used as biological repeats. Following incubation with the conditioned media, we measured the number of LX-2 cells expressing a-SMA (Panel B) and the level of expression of a-SMA in the cells (FIG. 25 A). Since a-SMA is an activation marker of human hepatic stellate cells, a decrease in its level is indicative of reduced activation. As hepatic stellate cells are involved with liver scar generation, a decrease in their activation is indicative of a reduction in liver fibrosis. As can be seen in FIG. 25 B, LX-2 cells treated with RTX001 showed both a reduced number of a-SMA expressing cells and a lower expression of a-SMA, indicative of a repressive effect of RTX001 in activation of hepatic stellate cells.

### Example 19—Optimised Bicistronic mRNA Results in Improved IL-10 Secretion

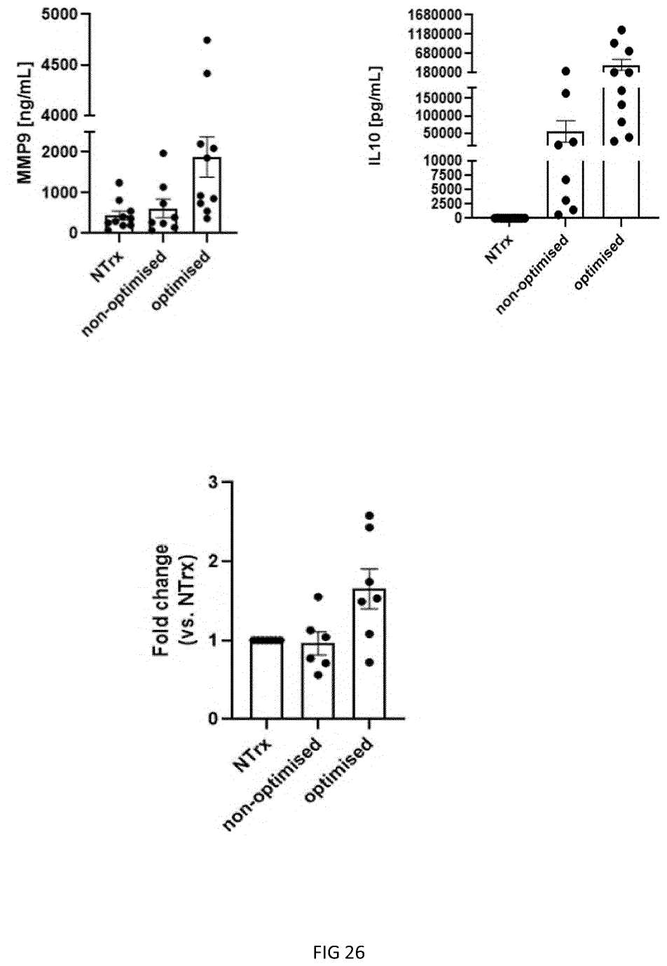

As can be seen in FIG. 26 right panel, transfection of the optimised bi-cistronic mRNA resulted in a much higher IL-10 secretion. Surprisingly, despite the inhibitory effect of IL-10 on MMP9 secretion, as seen in FIG. 1B, the optimised sequence resulted both in much higher MMP9 secretion and a much higher MMP activity as compared to non-transfected macrophage (NTRx) (left and lower panels of FIG. 26). In these experiments, the conditions and mRNA concentrations used were the same for optimised/non-optimised mRNAs.

### Example 20—Engineered Macrophages are Stable in an Inflammatory Environment

In the livers of patients with end stage chronic liver disease there is an accumulation of pro-inflammatory macrophages which secrete pro-inflammatory cytokines and elevate the inflammatory response (see for example Campana et al, 2021).

To confirm that the IL-10+MMP9 expressing macrophages will not revert to a pro-inflammatory phenotype once they are administered into the patient and encounter the inflammatory environment of the liver, we performed a stability study. In this study, we confirmed the stability of the pro-restorative phenotype of the IL-10+MMP9 expressing macrophages in an inflammatory environment (which mimics the environment in the liver of a patient suffering from end-stage chronic liver disease by modelling the level of hIFN-γ in patients).

To test stability of the phenotype, we tested the phenotype of the cells following incubation with hIFN-γ using the following experimental procedure:

To obtain macrophages, PBMCs were isolated from steady state leukapheresis or mobilised blood samples, CD14+ cells were isolated from PBMCs and plated for 5 days in TexMacs+M-CSF to differentiate into macrophages. On day 5 of culture, the macrophages were harvested and either transfected with IL-10+MMP9 or left un-transfected. After transfection, the macrophages were incubated overnight (^{˜}16 hrs) in TexMacs+100 ng/mL M-CSF, IL-4 (20 ng/mL and IL-13 (20 ng/mL) at 37° C. with 5% C02. After this rest period the macrophages were harvested and plated in U bottomed ultra-low adherence sterile culture plates (Corning Cat #7007) at a density of 2×105Cells/well in TexMacs either with or without stimulation (hIFN-γ). Concentrations of hIFN-γ were calculated with the following information: Total IFN-γ in the human liver was calculated using the concentration of total IFN-γ in injured mouse liver as determined by previous In Vivo Pharmacology experiments and scaling up to the weight of the human liver. This total level of IFN-γ was then divided by the planned therapeutic cell dose to provide the level of IFN-γ per cell. This was used to create a concentration gradient to account for the possibility of patients presenting with higher or lower levels of IFN-γ than the calculated concentration. All groups were plated to provide 3 technical replicates. The macrophages were then cultured for 24 hours before being centrifuged at 300×G for 5 minutes. The culture supernatant was collected and frozen at −20° C. and the cells were used for flow cytometry to assess the levels of CD80, CD86, MHCII, 25F9, CD14, CD206 and CD163 present on the cell surface. After staining cells were analysed on the Novocyte Quanteon and data analysis was performed using NovoExpress and GraphPad Prism software. No significant change was seen in proinflammatory markers (HLA-DR, CD80 and CD86) or macrophage identity marker CD206.

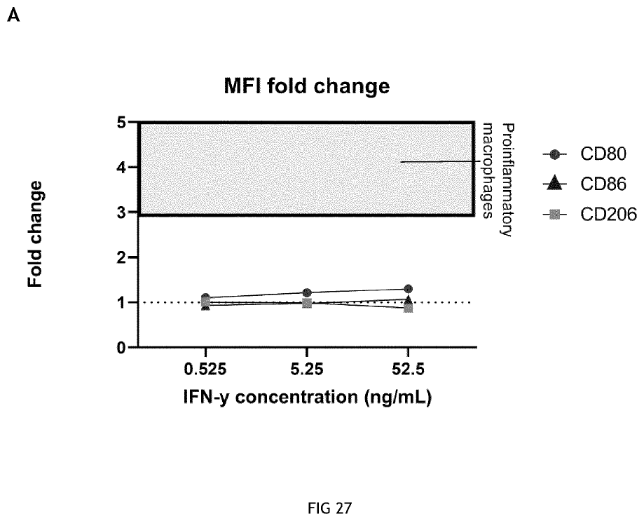

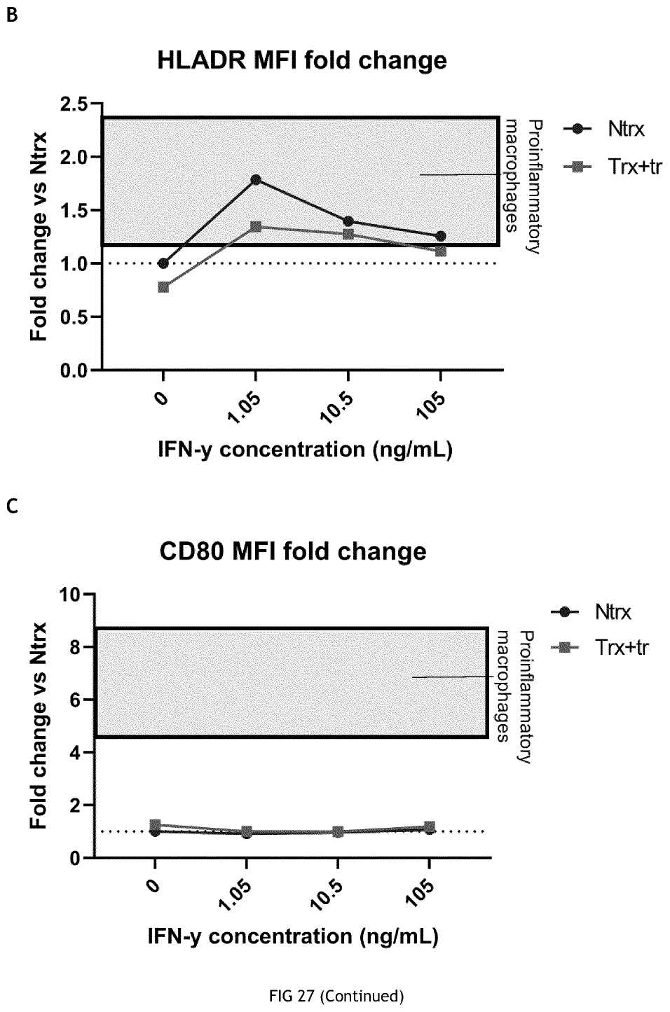

As can be seen in FIG. 27 the phenotype of cells expressing MMP9 and IL10 is stable in an inflammatory environment.

### Example 21—The Expression of Macrophage Markers Differs Between the Macrophage Products

The macrophage products consist of: non-transfected and untreated (UT) or treated (UT+TR) with IL-4, IL-13 and M-CSF; transfected with IL-10 and MMP9 and untreated (IL-10 MMP9 TRx) or treated with IL-4, IL-13 and M-CSF post-transfection (IL-10 MMP9 TRx+TR). Mean Fluorescence Intensity was measured by flow cytometry as described herein, and results are shown in FIG. 28.

### Example 22—Post-Transfection Treatment does not Contribute to Further Polarisation of the Engineered Macrophages, but does Improve Cryoresilience

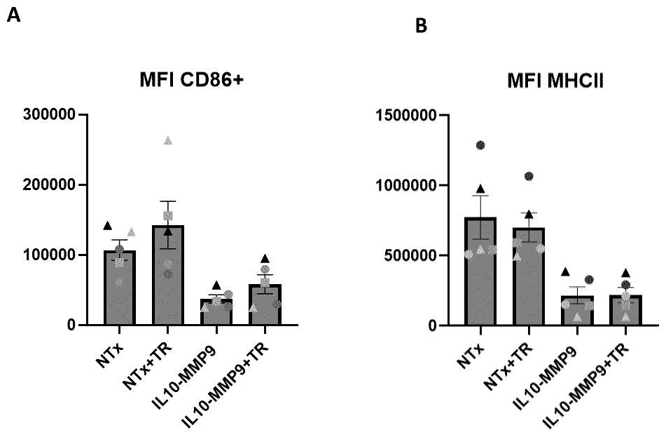

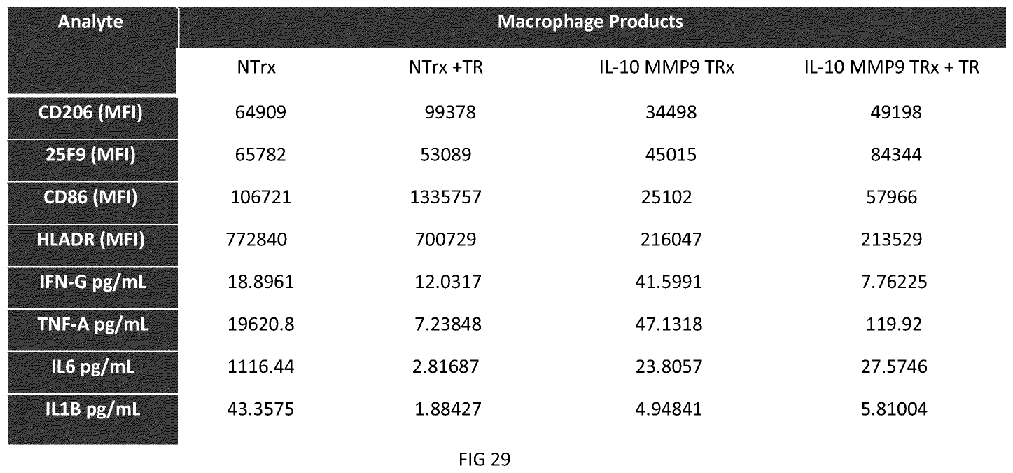

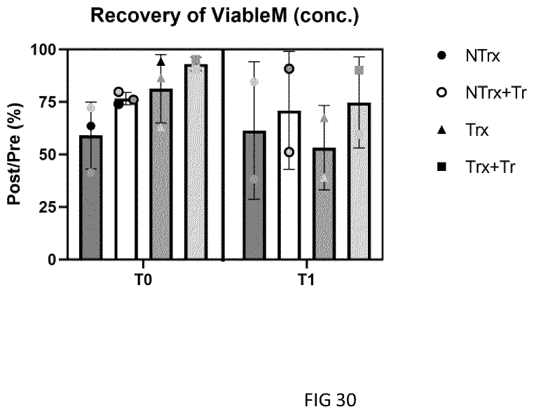

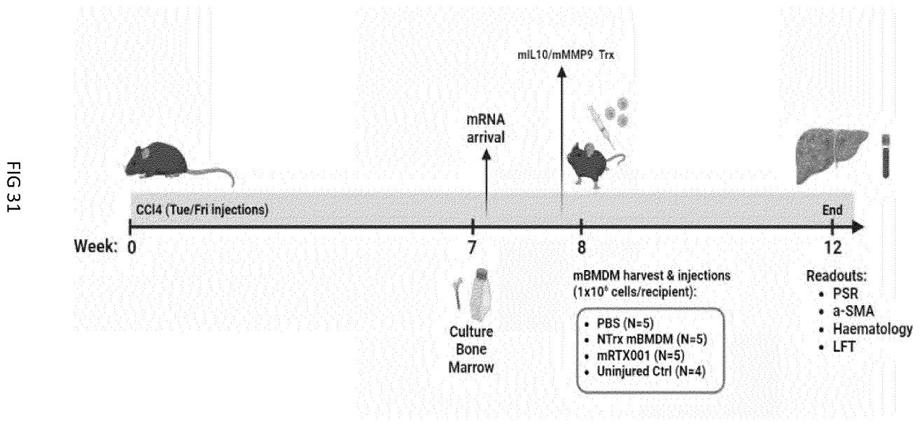

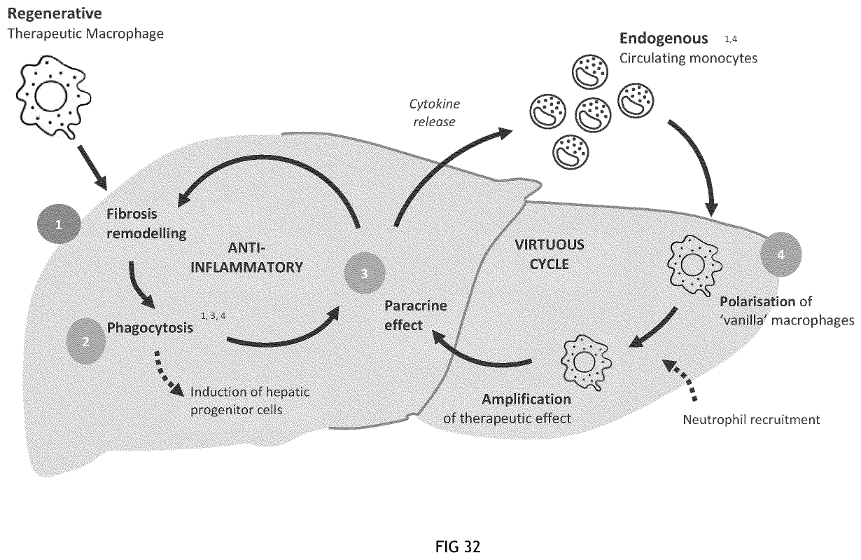

Expression of CD86 (A) and MHC II (B) was measured in cells transfected with the bicistronic construct encoding MMP9 and IL-10, without (IL-10-MMP9) or with further treatment with IL-4, IL-13 and M-CSF post-transfection (IL-10-MMP9+TR). No difference in the expression of CD86 and MHC II could be detected between the IL-10-MMP9 and IL-10-MMP9+TR groups (FIG. 29). Therefore, the transfected cells are completely self-polarising due to the secretion of IL-10 (e.g. the reduction in HLA-DR and CD86 is solely due to IL-10 secretion by the transfected cells). However, as shown in FIG. 30, the cryo-resilience of non-transfected (NTRx) or transfected (TRx) macrophages which have been incubated with IL-4+IL-13 is higher, as measured by the percentage of cells which remain viable post-cryopreservation.

### Example 23—Measuring the Anti-Fibrotic Performance of Engineered Macrophages in an Immunocompromised Mouse Model of Liver Fibrosis

The macrophages described herein are of human origin. To understand their behaviour in a mammalian system, immunocompromised mice are required that permit the transient engraftment of human material in vivo. Otherwise, immunocompetent wild-type recipient strains would result in the rapid elimination of administered human cells via mechanisms of xenogenic acute rejection. The highly immunocompromised ‘NSG’ strain is genetically altered to ablate host T cells, B cells, and NK cell activity. Without wishing to be bound by theory or mechanism, the mode-of-action (MoA) of the macrophages relies, in part, on recruitment of host innate immune effector cells, including monocytes (Thomas et al. 2011. Ma et al. 2017). Whilst NSG mice can accept transfer of human cells, they lack several host functional immune cells, which provide the secondary immunological response following the macrophage treatment. Consequently, the full pharmacological response, as would be anticipated in humans, cannot be modelled in immunocompromised strains. In addition, it is likely that a proportion of human proteins in the secretome of the macrophages may not be functional in the mouse due to species differences in receptor binding and downstream signalling pathways. Therefore, limitations in the immunocompromised strains will, at best, underestimate, or preclude altogether, absolute demonstration of efficacy (evidenced as a statistically significant reduction in liver fibrosis) with the macrophages described herein. Nevertheless, described below is an in vivo experimental model to test efficacy.

The macrophages used in this example are primary human monocyte-derived macrophage (hMDM) that have been phenotypically modified via transient transfection to deliver a bicistronic mRNA transcript encoding IL-10 and MMP-9 with a P2A self-cleaving peptide. In order to demonstrate the anti-fibrotic performance of the tested cell therapy, experimental liver fibrosis is modelled in an immunocompromised mouse strain. NSG mice (full nomenclature: NOD.Cg-PrkdcSCID Il2rgtmlWjl/SzJ, sourced from Charles River Laboratories) lack T-cells, B-cells, and NK-cells, which render the mouse immunodeficient and consequently allow the transient engraftment of human cells in vivo. Liver fibrosis is induced in NSG mice by twice-weekly administration of carbon tetrachloride (CCl4, i.p., 0.4 μL/g body weight, diluted in olive oil) for 12 weeks. The CCl4 fibrosis model is a well-recognised tractable rodent fibrosis model and has demonstrated clinical predictability with obeticholic acid (Fan et al., 20194⁴⁷; Younossi et al. 2019⁴⁸) and lanifibranor (Wettstein et al., 20174⁴⁹; Francque et al. 2021⁵⁰). The anti-fibrotic performance of the tested macrophages is tested by delivering the cells (1×10⁶) intravenously after 8 weeks of CCl4 administration when liver fibrosis is established. Injured mice that receive PBS alone serve as vehicle controls. In addition, injured mice that receive non-transfected human monocyte-derived macrophages (NTrx hMDMs, 1×10⁶, i.v.) serve as a comparator group to control for the engineering step. Cells, or vehicle alone, are administered (100 μL, i.v., in PBS) 24 hours after the 17th dose of CCl4. All mice continue to receive CCl4 for four additional weeks. All mice are humanely euthanised 24 hours after the 24th and final dose of CCl4 via exsanguination before cervical dislocation under terminal anaesthesia. Whole blood is collected via cardiac puncture and processed to liberate plasma or serum for liver chemistry biomarker evaluation. The liver, spleen, lung, heart, and kidneys are harvested and fixed for histological analysis. To determine the anti-fibrotic performance of the macrophages, liver fibrosis is quantified in histological liver sections using picrosirius red (PSR) staining to visualise collagen fibres. PSR stained sections are digitised via a microscope slide scanner (Zeiss Axioscan, Zeiss AG) and liver fibrosis is quantified by image analysis software (Zen, Zeiss AG). The anti-fibrotic performance of the macrophages is evaluated by comparing the percentage of PSR in the macrophage-treated mouse group compared to mice that received vehicle alone or NTrx-hMDM treatment.

### Example 24—Measuring the Anti-Fibrotic Performance of Engineered Macrophages in an Immunocompromised Mouse Model of Fibrotic Fatty Liver Disease

In order to demonstrate the anti-fibrotic performance of the engineered macrophages of Example 23, experimental liver fibrosis is modelled in an immunocompromised mouse strain on a background of fatty liver disease. NSG mice (full nomenclature: NOD.Cg-PrkdcSCID Il2rgtmlWjl/SzJ, sourced from Charles River Laboratories) lack T-cells, B-cells, and NK-cells, which render the mouse immunodeficient and consequently allow the transient engraftment of human cells in vivo. Liver fibrosis is induced by feeding mice on a choline-deficient amino acid-defined high-fat diet (CDAA HFD) with restricted methionine content. Ad libitum feeding of CDAA HFD induces hepatic steatosis and inflammation resulting in a distinct fibrotic histological pattern but without exhibiting profound weight loss in the animals. The CDAA HFD model is valuable because, unlike other fibrosis models, CDAA HFD-induced fibrosis resolves slowly exhibiting stable fibrosis for at least two weeks following the cessation of the dietary insult. Therefore, the characteristics of the model allows evaluation of test articles in the absence of ongoing injury. Here, NSG mice are provided CDAA-HFD (ad libitum) for 12 weeks to induce established liver fibrosis on a background of fatty liver disease. After 12 weeks, mice are switched back onto standard chow diet. Following resumption of normal diet, the engineered macrophages are tested for their anti-fibrotic performance following intravenous administration. Injured mice receive PBS alone serving as vehicle controls. In addition, injured mice that receive non-transfected human monocyte-derived macrophages (NTrx hMDMs, 1×10⁶, i.v.) serve as a comparator group to control for the engineering step. Cells, or vehicle alone, are administered (100 piL, i.v., in PBS) 24 hours after the resumption of standard (RM3) diet. All mice continue to receive standard diet for four additional weeks. At the end of study, all mice are humanely euthanised via exsanguination before cervical dislocation under terminal anaesthesia. Whole blood is collected via cardiac puncture and processed to liberate plasma or serum to evaluate liver chemistry biomarkers. The liver, spleen, lung, heart, and kidneys, are harvested and fixed for histological analysis. To measure liver fibrosis, histological liver sections are stained using picrosirius red (PSR) staining to visualise collagen fibres. PSR stained sections are digitised via a microscope slide scanner (Zeiss Axioscan, Zeiss AG) and liver fibrosis is quantified by image analysis software (QuPath Image Analysis open source software). The anti-fibrotic performance of the engineered macrophages is evaluated by comparing the percentage of PSR-positive staining in macrophage-treated mouse samples versus samples derived from mice that received vehicle alone, or NTrx-hMDM treatment.

### Example 25—Use of Murine Macrophages Engineered as a Surrogate of Human Engineered Macrophages in an Immunocompetent Model of Liver Fibrosis

Table 4. Exemplary structural and functional properties of the engineered macrophages

| | Readout for RTX001 (normalised to |
|---|---|
| Therapeutically Relevant | MATCH-like cells as described in |
| Feature | WO2019175595) |
| CD86 | ≥2-fold reduction |
| HLA-DR | ≥2-fold reduction |
| Ability of secreted media to | ≥1.5 fold increase in CD206 and CD163 and |
| polarize neighboring | ≥1.5 fold decrease of CD86 and HLA-DR |
| macrophages in-vitro | |
| Monocyte Migration | ≥2 times monocyte recruitment |
| IL-10 secretion | 1000-fold increase |
| MMP3 secretion | 10-fold increase |
| MMP10 secretion | 20-fold increase |

TABLE 5
Further exemplary structural and functional
properties of the engineered macrophages

| Therapeutically Relevant Feature | Readout |
|---|---|
| IL10 secretion | >10,000 pg/ml |
| MMP9 secretion | >200 ng/ml |
| MMP activity | >1.5 fold vs untransfected |
| | macrophages (NTrx) |
| CD206 | >5 fold vs D 0 (monocytes at day 0 |
| | of culture as described herein) |
| 25F9 | > 5 fold vs D 0 (monocytes at day 0 |
| | of culture as described herein) |
| CD80 | <10% vs untransfected |
| | macrophages (NTrx) |
| TNFα | <40 pg/ml |

### CONCLUSIONS

In conclusion, the genetic engineering of IL-10 in combination with MMP9 delivers a number of features and functions desirable for a cell therapy, e.g. for preventing/treating inflammatory conditions and/or organ damage regeneration. IL-10 in combination of MMP9 delivers:
A solid macrophage identity, not perturbed by the engineering process.
A strong anti-inflammatory phenotype.
Ability to pattern naïve macrophages towards a pro-restorative phenotype.
Excellent phagocytic capacity.
Reassuring safety and biodistribution profile, including infiltration in the damaged organ and rapid clearance/absence in other organs.

The above features are shared with macrophages engineered with IL-10 only. However, the combination of IL-10+MMP9 delivers some specific and surprising features, key to deliver our desired therapeutic effect, such as:
A strong ability to attract monocytes, to then be patterned to a pro-restorative phenotype.
Ability to restore the MMP activity (surrogate for fibrosis/ECM remodelling) abrogated by the engineering of IL-10 alone.

Therefore, we believe that engineering a macrophage with a combination of IL-10 and MMP9 will deliver an efficacious product able to have both anti-inflammatory and anti-fibrotic functions in several organ damage settings, both acute and chronic (remodelling of ECM components is paramount in acute damage setting, too, to ensure the restitutio ad integrum of the tissue and proper regeneration).

1. An engineered macrophage engineered to overexpress IL-10.

2. The engineered macrophage of claim 1, wherein the macrophage secretes IL-10 at a culture supernatant concentration of at least 10,000 pg/ml when cultured in vitro at a cell concentration of 4×10⁶/ml.

3. The engineered macrophage of claim 1 or claim 2, wherein the macrophage is additionally engineered to overexpress MMP9.

4. The engineered macrophage of claim 3, wherein the engineered macrophage comprises an exogenous coding sequence for IL-10 and an exogenous coding sequence for MMP9.

5. The engineered macrophage as claimed in claim 4, wherein expression of said exogenous coding sequences has a synergistic effect in restoring MMP activity when compared to engineered macrophages comprising an exogenous sequence for IL-10 alone and/or a synergistic effect in monocyte recruitment by the macrophages.

6. The engineered macrophage of any one of claims 1-5, wherein said macrophage and/or coding sequences are human.

7. The engineered macrophage of any one of claims 4-6, wherein the exogenous coding sequence for IL-10 encodes a protein with an amino acid sequence at least 85%, at least 90%, at least 95%, 96%, 97%, 98% or 99% identical to SEQ ID NO: 4, optionally wherein the IL-10 protein comprises an amino acid sequence identical to SEQ ID NO: 4.

8. The engineered macrophage of any one of claims 4-7, wherein the exogenous coding sequence for MMP9 encodes a protein with an amino acid sequence at least 85%, at least 90%, at least 95%, 96%, 97%, 98% or 99% identical to SEQ ID NO: 6, optionally wherein the IL-10 protein comprises an amino acid sequence identical to SEQ ID NO: 6.

9. The engineered macrophage of any one of claims 4, 5, 7 or 8 wherein said exogenous coding sequences are present on one or more nucleic acid molecules or are integrated into the genome of said macrophage.

10. The engineered macrophage of claim 9 wherein said nucleic acid molecule(s) are DNA or RNA molecules, preferably mRNA molecules, optionally wherein the IL-10 and MMP9 are expressed from the same mRNA molecule, further optionally wherein the mRNA molecule encodes IL-10 and MMP9 linked by a linker sequence, further optionally wherein the linker is a self-cleaving 2A linker, further optionally wherein the linker is p2A.

11. The engineered macrophage of claim 10, wherein the nucleic acid molecule(s) are mRNA molecule(s), comprising a sequence at least 85%, at least 90%, at least 95%, at least 96%, at least 97%, at least 98% or at least 99% identical to SEQ ID NO: 13, optionally wherein the nucleic acid comprises SEQ ID NO: 13.

12. The engineered macrophage of claims 10 or 11, wherein the nucleic acid molecule(s) are mRNA molecule(s), comprising a sequence at least 85%, at least 90%, at least 95%, at least 96%, at least 97%, at least 98% or at least 99% identical to SEQ ID NO: 14, optionally wherein the nucleic acid comprises SEQ ID NO: 14.

13. The engineered macrophage of claims 10-12, wherein the nucleic acid molecule is an mRNA molecule which encodes IL-10 and MMP9 linked by a linker sequence, and wherein the linker sequence encodes a protein comprising an amino acid sequence as described in SEQ ID NO: 7, optionally wherein the protein encoded by the linker sequence comprises an amino acid sequence as described in SEQ ID NO: 9.

14. The engineered macrophage of claims 10-13, wherein the nucleic acid molecule is an mRNA molecule which encodes IL-10 and MMP9 linked by a linker sequence, and wherein the linker sequence comprises mRNA with a sequence as described in SEQ ID NO: 15.

15. The engineered macrophage of claims 10-14, wherein the nucleic acid molecule is an mRNA molecule comprising a sequence at least 80% identical to SEQ ID NO: 10, preferably at least 85%, at least 90%, at least 95%, at least 96%, at least 97%, at least 98%, or at least 99% identical to SEQ ID NO: 10, optionally wherein the mRNA further comprises a polyA tail between 65 and 250 residues long, preferably 90 to 120 residues long, preferably about, and/or a 5′ cap.

16. The engineered macrophage of claims 10-15, wherein the nucleic acid molecule is an mRNA molecule comprising a sequence at least 80% identical to SEQ ID NO: 16, preferably at least 85%, at least 90%, at least 95%, at least 96%, at least 97%, at least 98%, or at least 99% identical to SEQ ID NO: 16, optionally wherein the mRNA further comprises a 5′ cap.

17. The engineered macrophage of claim 16 wherein the mRNA molecules contain chemically modified residues, preferably modified uracil residues, and optionally at least one synthetic cap.

18. The engineered macrophage of any of claims 4 to 17, wherein the exogenous coding sequence for IL-10 is on the same nucleic acid as the exogenous coding sequence for MMP9.

19. The engineered macrophage according to any of the preceding claims, wherein the macrophage is engineered by editing the endogenous promoters of the IL-10 gene and/or the MMP9 gene, or wherein the macrophage is engineered by modulating the expression of an endogenous silencing RNA, or introducing an exogenous silencing RNA sequence, optionally wherein the silencing RNA is miRNA.

20. The engineered macrophage according to any preceding claim, wherein the level of metalloproteinase activity is at least 1.5 times the metalloproteinase activity of a non-engineered macrophage.

21. The engineered macrophage of any one of the preceding claims wherein the engineered macrophage has an at least two-fold reduced expression of CD86 compared to non-engineered, non-polarised cells.

22. The engineered macrophage of any one of the preceding claims wherein the engineered macrophage has an at least two-fold reduced expression of HLA-DR compared to non-engineered, non-polarised cells.

23. The engineered macrophage of any one of the preceding claims wherein the engineered macrophage has an at least 1000-fold increased secretion of IL-10 compared to non-engineered, non-polarised cells.

24. The engineered macrophage of any one of the preceding claims wherein the engineered macrophage has an at least 10-fold increased secretion of MMP3 compared to non-engineered, non-polarised cells.

25. The engineered macrophage of any one of the preceding claims wherein the engineered macrophage has an at least 20-fold increased secretion of MMP10 compared to non-engineered, non-polarised cells.

26. The engineered macrophage of any one of the preceding claims wherein the macrophage secretes IL-10 at a culture supernatant concentration of at least 10,000 pg/ml when cultured in vitro at a cell concentration of 4×10⁶/ml.

27. The engineered macrophage of any one of the preceding claims wherein the macrophage secretes MMP9 at a culture supernatant concentration of at least 200 ng/ml when cultured in vitro at a cell concentration of 4×10⁶/ml.

28. The engineered macrophage of any one of the preceding claims wherein the engineered macrophage has an at least 5-fold increased expression of CD206 compared to monocytes.

29. The engineered macrophage of any one of the preceding claims wherein the engineered macrophage has an at least 5-fold increased expression of 25F9 compared to monocytes.

30. The engineered macrophage of any one of the preceding claims wherein the engineered macrophage has an at least ten percent reduced expression of CD80 compared to non-engineered, non-polarised cells.

31. The engineered macrophage of any one of the preceding claims wherein the macrophage secretes TNF-α at a culture supernatant concentration of up to 40 pg/ml when cultured in vitro at a cell concentration of 4×10⁶/ml.

32. The engineered macrophage of any one of the preceding claims, wherein the engineered macrophage has phagocytic ability at least equivalent to a non-engineered, non-polarised cell.

33. The engineered macrophage according to any one of claims 1-32 wherein the metalloproteinase activity is restored relative to the reduced metalloproteinase activity in a macrophage engineered with IL-10 coding sequence alone.

34. The engineered macrophage according to any preceding claim, wherein said macrophage is transiently transfected, optionally via electroporation.

35. The engineered macrophage of claim 34, wherein the transfection is non-viral.

36. The engineered macrophage of any preceding claim wherein said macrophage has a pro-restorative phenotype.

37. A population of engineered macrophages according to any preceding claim.

38. A therapeutic composition comprising a population of macrophages according to claim 37 plus a pharmaceutically acceptable medium.

39. An engineered macrophage of any one of claims 1 to 36, a population of macrophages of claim 37, or a composition of claim 38, for use in therapy.

40. An engineered macrophage, population or composition according to claim 39 wherein said therapy is administered to a subject in need thereof.

41. An engineered macrophage of any one of claims 1 to 36, a population of macrophages of claim 37, or a composition of claim 38, for use in treating an inflammatory condition in a subject.

42. An engineered macrophage, population or composition according to claims 40 or 41, wherein said macrophages are autologous or allogenic to the subject.

43. An engineered macrophage, population or composition according to claim 41, wherein the inflammatory condition is a liver injury, optionally chronic liver injury.

44. An engineered macrophage, population or composition according to claim 41 or 43, wherein the condition is a chronic inflammatory condition with a fibrotic element, optionally wherein the condition is organ damage associated with chronic inflammation.

45. The engineered macrophage, population or composition according to any one of claims 41, 43 or 44, wherein the condition is fibrosis, and wherein fibrosis is in or affects an organ selected from the group consisting of: liver, lung, heart, kidney, pancreas, skin, gastrointestinal, bone marrow, hematopoietic tissue, nervous system, eye and a combination thereof.

46. The engineered macrophage, population or composition of any of claims 43-45, wherein the condition is liver cirrhosis.

47. The engineered macrophage, population or composition of claim 46, wherein the liver cirrhosis resulted from at least one disease or condition selected from the group consisting of: non-alcoholic fatty liver disease (NAFL) (e.g., non-alcoholic fatty liver disease (NAFLD) or non-alcoholic steatohepatitis (NASH)), alcoholic liver disease (e.g., alcoholic fatty liver disease (AFLD) or alcoholic steatohepatitis (ASH)), mechanical trauma to the liver, biliary obstruction, autoimmune hepatitis, iron overload, Hepatitis B infection (HBV) and Hepatitis C infection (HCV).

48. An engineered macrophage population or composition of claim 46, wherein the liver cirrhosis resulted from steatotic liver disease (SLD), optionally wherein the steatotic liver disease is metabolic dysfunction-associated steatotic liver disease, metabolic-associated steatohepatitis, Met-ALD or Cryptogenic SLD.

49. The engineered macrophage, population or composition of claims 46-48, wherein the liver cirrhosis is selected from compensated cirrhosis and decompensated cirrhosis.

50. An engineered macrophage, population or composition according to any of claims 41-49 wherein the condition is acute-on-chronic liver failure (ACLF).

51. An engineered macrophage, population or composition according to claims 46-49, for use in treating a subject that has recovered from their first hepatic decompensation event (recompensated), optionally wherein the first hepatic decompensation event required the subject's hospitalization, preferably wherein the subject has not undergone an additional hepatic decompensation event after having recovered from the first decompensation event.

52. The engineered macrophage, population or composition according to claim 46-49 and 51, wherein the subject exhibits or has recovered from one or more clinical signs of hepatic decompensation selected from the list consisting of jaundice, ascites, hepatic encephalopathy, hepatorenal syndrome, variceal haemorrhage and gastrointestinal haemorrhage.

53. The engineered macrophage, population or composition according to any of claims 1-52 wherein the macrophages are derived from human monocyte-derived macrophages (hMDMs) or stem cells, optionally wherein the stem cells are induced pluripotent stem cells (iPSCs).

54. The engineered macrophage, population or composition according to claim 53, wherein the macrophages are derived from iPSCs, and the iPSCs are essentially devoid of functional HLA I and II complexes on their surface.

55. A method of improving the migration of monocytes to an area of inflammation comprising the use of an engineered macrophage, population of engineered macrophages or a composition according to any of the preceding claims.

56. A method according to claim 55, wherein the method polarizes the host monocytes/macrophages to a pro-restorative phenotype and/or away from a pro-inflammatory phenotype.

57. A method of producing an engineered macrophage according to any one of claims 1 to 36, comprising transiently transfecting a macrophage with an mRNA molecule encoding IL-10 and/or MMP9.

58. A method according to claim 57, comprising contacting the macrophage with IL-4, IL-13 and M-CSF before, during or after the transfection.

59. A method according to claims 57 or 58, wherein the mRNA molecules encoding IL-10 and MMP9 are co-transfected using a bi-cistronic vector, linked by a p2A linker sequence.

60. An engineered macrophage according to any one of claims 1 to 36, wherein the macrophage is engineered with a mRNA construct encoding a human IL-10 fused to a human MMP9 protein via a cleavable linker.

61. A method of treating inflammation and/or fibrosis comprising administering to a subject in need thereof a therapeutically effective amount of engineered macrophages according to any one of claims 1-36.

62. A method of polarising macrophages to a pro-restorative phenotype, wherein the polarised macrophages have an increased expression of CD163 and CD206, and a reduced expression of HLA DR and CD86 compared to cells not polarised to a pro-restorative phenotype, wherein the method comprises engineering the macrophage to express IL-10 and MMP9 above endogenous levels.

63. The method of claim 62, wherein the macrophage is engineered to express IL-10 and MMP9 by introducing exogenous nucleic acid comprising nucleotide sequences encoding IL-10 and MMP9.

64. The method of claim 63, wherein the nucleotide sequences encoding IL-10 and MMP9 are present on the same nucleic acid molecule.

65. The method of claim 63, wherein the nucleotide sequences encoding IL-10 and MMP9 are present on separate nucleic acid molecules.

66. The method of any one of claims 63-65, wherein the nucleic acid is mRNA.

67. A method of polarising macrophages to a pro-restorative phenotype, wherein the polarised macrophages have an increased expression of CD163 and CD206, and a reduced expression of HLA DR and CD86 compared to cells not polarised to a pro-restorative phenotype, wherein the method comprises engineering the macrophage to overexpress IL-10, optionally wherein the macrophage secretes IL-10 at a culture supernatant concentration of at least 10,000 pg/ml when cultured in vitro at a cell concentration of 4×10⁶/ml.

68. The method of claim 67, wherein the macrophage is engineered to express IL-10 by introducing exogenous nucleic acid comprising a nucleotide sequence encoding IL-10

69. The method of claim 67 or 68, wherein the nucleic acid is mRNA.

70. A method of improving cryoresilience in macrophages, comprising incubating the macrophages in medium comprising IL-4, IL-13 and M-CSF.

71. A method of cryopreserving macrophages, comprising incubating the macrophages in medium comprising IL-4, IL-13 and M-CSF prior to cryopreservation.

72. The method of claim 70 or 71, wherein the concentration of IL-4 and IL-13 in the medium are 20 ng/ml, the concentration of M-CSF is 100 ng/ml, and the macrophages are at a concentration of 4×10⁶ cells/ml.

73. The method of claims 70-72, wherein the cells are incubated overnight in the medium comprising IL-4, IL-13 and M-CSF.

74. Cryopreserved macrophages obtained by a method of any of claims 70-73.


SEQUENCE LISTING (20 sequences)

SEQ ID NO: 1 - RNA, 537 nt, Homo sapiens
  source 1..537: mol_type = mRNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 2 - RNA, 2724 nt, synthetic construct
  source 1..2724: mol_type = other RNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacggaagcggagccacgaacttctctctgttaaagcaagcaggagatgttgaagaaaaccccgggcctatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 3 - RNA, 2124 nt, Homo sapiens
  source 1..2124: mol_type = mRNA
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 4 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - protein, 907 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSGATNFSLLKQAGDVEENPGPMSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 6 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 7 - protein, 8 aa, synthetic construct
DXEXNPGP

SEQ ID NO: 8 - RNA, 66 nt, synthetic construct
  source 1..66: mol_type = other RNA
ggaagcggagccacgaacttctctctgttaaagcaagcaggagatgttgaagaaaaccccgggcct

SEQ ID NO: 9 - protein, 22 aa, synthetic construct
GSGATNFSLLKQAGDVEENPGP

SEQ ID NO: 10 - RNA, 2724 nt, synthetic construct
  source 1..2724: mol_type = other RNA
  modified_base 2: mod_base = mo5u
  modified_base 2: mod_base = mo5u
  modified_base 10: mod_base = mo5u
  modified_base 17: mod_base = mo5u
  modified_base 22: mod_base = mo5u
  modified_base 25: mod_base = mo5u
  modified_base 29: mod_base = mo5u
  modified_base 32: mod_base = mo5u
  modified_base 35: mod_base = mo5u
  modified_base 38: mod_base = mo5u
  modified_base 47: mod_base = mo5u
  modified_base 76: mod_base = mo5u
  modified_base 88: mod_base = mo5u
  modified_base 97: mod_base = mo5u
  modified_base 98: mod_base = mo5u
  modified_base 108: mod_base = mo5u
  modified_base 110: mod_base = mo5u
  modified_base 119: mod_base = mo5u
  modified_base 122: mod_base = mo5u
  modified_base 131: mod_base = mo5u
  modified_base 142: mod_base = mo5u
  modified_base 143: mod_base = mo5u
  modified_base 145: mod_base = mo5u
  modified_base 152: mod_base = mo5u
  modified_base 160: mod_base = mo5u
  modified_base 161: mod_base = mo5u
  modified_base 163: mod_base = mo5u
  modified_base 164: mod_base = mo5u
  modified_base 170: mod_base = mo5u
  modified_base 182: mod_base = mo5u
  modified_base 189: mod_base = mo5u
  modified_base 191: mod_base = mo5u
  modified_base 194: mod_base = mo5u
  modified_base 197: mod_base = mo5u
  modified_base 209: mod_base = mo5u
  modified_base 212: mod_base = mo5u
  modified_base 220: mod_base = mo5u
  modified_base 221: mod_base = mo5u
  modified_base 229: mod_base = mo5u
  modified_base 233: mod_base = mo5u
  modified_base 238: mod_base = mo5u
  modified_base 248: mod_base = mo5u
  modified_base 250: mod_base = mo5u
  modified_base 257: mod_base = mo5u
  modified_base 260: mod_base = mo5u
  modified_base 265: mod_base = mo5u
  modified_base 266: mod_base = mo5u
  modified_base 268: mod_base = mo5u
  modified_base 272: mod_base = mo5u
  modified_base 281: mod_base = mo5u
  modified_base 284: mod_base = mo5u
  modified_base 314: mod_base = mo5u
  modified_base 326: mod_base = mo5u
  modified_base 335: mod_base = mo5u
  modified_base 347: mod_base = mo5u
  modified_base 356: mod_base = mo5u
  modified_base 362: mod_base = mo5u
  modified_base 368: mod_base = mo5u
  modified_base 376: mod_base = mo5u
  modified_base 385: mod_base = mo5u
  modified_base 386: mod_base = mo5u
  modified_base 394: mod_base = mo5u
  modified_base 406: mod_base = mo5u
  modified_base 416: mod_base = mo5u
  modified_base 425: mod_base = mo5u
  modified_base 436: mod_base = mo5u
  modified_base 437: mod_base = mo5u
  modified_base 446: mod_base = mo5u
  modified_base 461: mod_base = mo5u
  modified_base 463: mod_base = mo5u
  modified_base 473: mod_base = mo5u
  modified_base 481: mod_base = mo5u
  modified_base 482: mod_base = mo5u
  modified_base 488: mod_base = mo5u
  modified_base 490: mod_base = mo5u
  modified_base 491: mod_base = mo5u
  modified_base 494: mod_base = mo5u
  modified_base 499: mod_base = mo5u
  modified_base 503: mod_base = mo5u
  modified_base 511: mod_base = mo5u
  modified_base 515: mod_base = mo5u
  modified_base 521: mod_base = mo5u
  modified_base 527: mod_base = mo5u
  modified_base 2722: mod_base = mo5u
atgcacagctccgccctgctgtgctgcctggtgctgctgaccggcgtgcgggccagccccggccagggcacacagtccgagaacagctgcacccacttcccaggcaatctccccaacatgctgagagacctgagggacgccttctcccgcgtgaagacattcttccagatgaaggaccagctggacaatctcctgctgaaggagagcctgctggaggacttcaagggctacctgggctgccaggccctgtccgagatgatccagttctacctggaggaggtgatgccacaggccgagaaccaggaccccgacatcaaggcccacgtgaacagcctgggcgagaacctgaagaccctgcggctgagactgaggcgctgccaccggttcctgccatgcgagaacaagtccaaggccgtggaacaagtgaagaacgccttcaacaagctgcaggagaagggcatctacaaggccatgagcgagttcgacatcttcatcaactacatcgaggcctacatgacaatgaagatcagaaacggctccggcgccaccaacttcagcctgctgaagcaggccggcgacgtggaggagaaccccggcccaatgtccctgtggcagcccctggtgctggtgctgctggtgctgggctgctgcttcgccgccccaaggcagcgccagagcacactggtgctgttccccggcgatctccggaccaacctgacagacagacagctggccgaggagtacctgtacaggtacggctacacccgcgtggccgagatgcggggcgagtccaagagcctgggcccagccctgctgctgctccagaagcagctgtccctgcccgagacaggcgagctggacagcgccaccctgaaggccatgagaacaccaaggtgcggcgtgcccgacctgggccgcttccagaccttcgagggcgacctgaagtggcaccaccacaacatcacatactggatccagaactactccgaggatctcccacgggccgtgatcgacgacgccttcgccagagccttcgccctgtggagcgccgtgacccccctgacattcaccagggtgtactcccgcgacgccgacatcgtgatccagttcggcgtggccgagcacggcgacggctacccattcgacggcaaggacggcctgctggcccacgccttccccccaggccccggcatccagggcgacgcccacttcgacgacgacgagctgtggagcctgggcaagggcgtggtggtgccaacacggttcggcaacgccgacggcgccgcctgccacttccccttcatcttcgagggcagatcctacagcgcctgcaccacagacgggcggtccgacggcctgccatggtgcagcaccacagccaactacgacaccgacgaccgcttcggcttctgcccctccgagcggctgtacacacaggacggcaacgccgacggcaagccatgccagttccccttcatcttccagggccagagctactccgcctgcaccacagacggcagaagcgacggctacaggtggtgcgccaccacagccaactacgaccgcgacaagctgttcggcttctgcccaacccgggccgactccacagtgatgggcggcaacagcgccggcgagctgtgcgtgttccccttcaccttcctgggcaaggagtactccacatgcaccagcgagggcagaggcgacggcaggctgtggtgcgccacaacctccaacttcgacagcgacaagaagtggggcttctgcccagaccagggctactccctgttcctggtggccgcccacgagttcggccacgccctgggcctggaccacagctccgtgcccgaggccctgatgtacccaatgtaccgcttcacagagggccccccactgcacaaggacgacgtgaacggcatccggcacctgtacggccccagaccagagcccgagccaaggcccccaaccacaaccacaccccagccaaccgcccccccaacagtgtgccccaccggcccacccacagtgcacccaagcgagcgccccaccgccggcccaacaggccccccatccgccggccccaccggcccacccacagccggcccaagcaccgccaccaccgtgcccctgtccccagtggacgacgcctgcaacgtgaacatcttcgacgccatcgccgagatcggcaaccagctgtacctgttcaaggacggcaagtactggcggttcagcgagggcagaggctccaggccccagggcccattcctgatcgccgacaagtggcccgccctgccacgcaagctggacagcgtgttcgaggagcggctgtccaagaagctgttcttcttcagcggcagacaggtgtgggtgtacaccggcgcctccgtgctgggccccaggcgcctggacaagctgggcctgggcgccgacgtggcccaggtgaccggcgccctgcggagcggcagaggcaagatgctgctgttctccggcaggcgcctgtggcggttcgacgtgaaggcccagatggtggaccccagaagcgcctccgaggtggacaggatgttccccggcgtgcccctggacacccacgacgtgttccagtaccgcgagaaggcctacttctgccaggaccggttctactggagagtgagctccaggagcgagctgaaccaggtggaccaggtgggctacgtgacctacgacatcctgcagtgccccgaggactaa

SEQ ID NO: 11 - protein, 907 aa, synthetic construct; identical to SEQ ID NO: 5

SEQ ID NO: 12 - RNA, 2793 nt, synthetic construct
  source 1..2793: mol_type = other RNA
atgcccggcagcgccctgctgtgctgcctgctgctgctgaccggaatgagaatctccagggggcagtacagccgggaggacaacaactgcacacacttcccagtgggccagtcccacatgctgctggaactgagaaccgccttcagccaggtgaagacattcttccagaccaaagaccagctggacaacatcctgctgacagactccctgatgcaggacttcaagggatacctggggtgccaggccctgagcgagatgatccagttctacctggtggaagtgatgccccaggccgagaaacacggcccagaaatcaaggagcacctgaactccctgggagaaaaactgaagaccctgaggatgcggctgagaaggtgccaccggttcctgccctgcgagaacaaaagcaaggccgtggaacaggtgaaatccgacttcaacaagctgcaggaccagggggtgtacaaagccatgaacgagttcgacatcttcatcaactgcatcgaagcctacatgatgatcaagatgaaaagcggctccggagccacaaacttcagcctgctgaagcaggccggggacgtggaggaaaacccaggccccatgtccccatggcagcccctgctgctggccctgctggccttcggatgcagctccgccgccccataccagagacagcccaccttcgtggtgttcccaaaagacctgaagacaagcaacctgaccgacacacagctggccgaggcctacctgtacaggtacgggtacacccgggccgcccagatgatgggcgaaaaacagtccctgagacccgccctgctgatgctgcagaagcagctgagcctgccacagacaggagagctggactcccagaccctgaaagccatcaggacaccccggtgcggggtgccagacgtgggcagattccagaccttcaagggactgaaatgggaccaccacaacatcacatactggatccagaactacagcgaagatctccccagggacatgatcgacgacgccttcgcccgggccttcgccgtgtggggggaggtggccccactgaccttcacaagagtgtacggccccgaagccgacatcgtgatccagttcggagtggccgagcacggggacggctacccattcgacggaaaggacgggctgctggcccacgccttccccccaggcgccggagtgcagggggacgcccacttcgacgacgacgaactgtggtccctgggcaaaggagtggtgatccccacctactacgggaacagcaacggcgccccatgccacttccccttcacattcgagggaaggtcctacagcgcctgcaccacagacgggcggaacgacggcaccccatggtgctccacaaccgccgactacgacaaggacggaaaattcgggttctgccccagcgaaagactgtacacagagcacggcaacggagaagggaagccatgcgtgttccccttcatcttcgagggaagatcctacagcgcctgcaccacaaaaggacggtccgacgggtacagatggtgcgccaccacagccaactacgaccaggacaagctgtacggcttctgcccaaccagggtggacgccacagtggtgggagggaacagcgccggcgaactgtgcgtgttccccttcgtgttcctgggaaaacagtactccagctgcacctccgacgggcggagagacggcaggctgtggtgcgccacaaccagcaacttcgacacagacaagaaatggggattctgcccagaccaggggtactccctgttcctggtggccgcccacgagttcggccacgccctgggactggaccacagctccgtgcccgaagccctgatgtacccactgtacagctacctggaggggttccccctgaacaaggacgacatcgacggcatccagtacctgtacggacgggggtccaaaccagaccccagaccacccgccaccacaaccacagaaccacagcccaccgccccacccacaatgtgcccaaccatccccccaacagcctaccccaccgtgggcccaacagtgggacccaccggggccccaagccccggcccaacatccagcccctccccaggacccaccggggccccaagccccggcccaacagcccccccaaccgccggatccagcgaggcctccacagaaagcctgtcccccgccgacaacccatgcaacgtggacgtgttcgacgccatcgccgagatccagggggccctgcacttcttcaaggacggctggtactggaaattcctgaaccacaggggaagccccctgcaggggccattcctgaccgcccggacatggcccgccctgccagccaccctggactccgccttcgaagacccccagacaaagagagtgttcttcttcagcggcaggcagatgtgggtgtacaccggaaaaacagtgctggggccacggtccctggacaagctgggcctgggacccgaggtgacccacgtgagcggcctgctgccaagaaggctgggaaaagccctgctgttctccaagggccgggtgtggagattcgacctgaaaagccagaaggtggacccccagtccgtgatcagggtggacaaagaattcagcggagtgccatggaacagccacgacatcttccagtaccaggacaaggcctacttctgccacggcaagttcttctggagagtgagcttccagaacgaggtgaacaaggtggaccacgaggtgaaccaggtggacgacgtgggctacgtgacctacgatctcctgcagtgcccctaa

SEQ ID NO: 13 - RNA, 534 nt, synthetic construct
  source 1..534: mol_type = other RNA
  modified_base 2: mod_base = mo5u
  modified_base 10: mod_base = mo5u
  modified_base 17: mod_base = mo5u
  modified_base 22: mod_base = mo5u
  modified_base 25: mod_base = mo5u
  modified_base 29: mod_base = mo5u
  modified_base 32: mod_base = mo5u
  modified_base 35: mod_base = mo5u
  modified_base 38: mod_base = mo5u
  modified_base 47: mod_base = mo5u
  modified_base 76: mod_base = mo5u
  modified_base 88: mod_base = mo5u
  modified_base 97: mod_base = mo5u
  modified_base 98: mod_base = mo5u
  modified_base 108: mod_base = mo5u
  modified_base 110: mod_base = mo5u
  modified_base 119: mod_base = mo5u
  modified_base 122: mod_base = mo5u
  modified_base 131: mod_base = mo5u
  modified_base 142: mod_base = mo5u
  modified_base 143: mod_base = mo5u
  modified_base 145: mod_base = mo5u
  modified_base 152: mod_base = mo5u
  modified_base 160: mod_base = mo5u
  modified_base 161: mod_base = mo5u
  modified_base 163: mod_base = mo5u
  modified_base 164: mod_base = mo5u
  modified_base 170: mod_base = mo5u
  modified_base 182: mod_base = mo5u
  modified_base 189: mod_base = mo5u
  modified_base 191: mod_base = mo5u
  modified_base 194: mod_base = mo5u
  modified_base 197: mod_base = mo5u
  modified_base 209: mod_base = mo5u
  modified_base 212: mod_base = mo5u
  modified_base 220: mod_base = mo5u
  modified_base 221: mod_base = mo5u
  modified_base 229: mod_base = mo5u
  modified_base 233: mod_base = mo5u
  modified_base 238: mod_base = mo5u
  modified_base 248: mod_base = mo5u
  modified_base 250: mod_base = mo5u
  modified_base 257: mod_base = mo5u
  modified_base 260: mod_base = mo5u
  modified_base 265: mod_base = mo5u
  modified_base 266: mod_base = mo5u
  modified_base 268: mod_base = mo5u
  modified_base 272: mod_base = mo5u
  modified_base 281: mod_base = mo5u
  modified_base 284: mod_base = mo5u
  modified_base 314: mod_base = mo5u
  modified_base 326: mod_base = mo5u
  modified_base 335: mod_base = mo5u
  modified_base 347: mod_base = mo5u
  modified_base 356: mod_base = mo5u
  modified_base 362: mod_base = mo5u
  modified_base 368: mod_base = mo5u
  modified_base 376: mod_base = mo5u
  modified_base 385: mod_base = mo5u
  modified_base 386: mod_base = mo5u
  modified_base 394: mod_base = mo5u
  modified_base 406: mod_base = mo5u
  modified_base 416: mod_base = mo5u
  modified_base 425: mod_base = mo5u
  modified_base 436: mod_base = mo5u
  modified_base 437: mod_base = mo5u
  modified_base 446: mod_base = mo5u
  modified_base 461: mod_base = mo5u
  modified_base 463: mod_base = mo5u
  modified_base 473: mod_base = mo5u
  modified_base 481: mod_base = mo5u
  modified_base 482: mod_base = mo5u
  modified_base 488: mod_base = mo5u
  modified_base 490: mod_base = mo5u
  modified_base 491: mod_base = mo5u
  modified_base 494: mod_base = mo5u
  modified_base 499: mod_base = mo5u
  modified_base 503: mod_base = mo5u
  modified_base 511: mod_base = mo5u
  modified_base 515: mod_base = mo5u
  modified_base 521: mod_base = mo5u
  modified_base 527: mod_base = mo5u
atgcacagctccgccctgctgtgctgcctggtgctgctgaccggcgtgcgggccagccccggccagggcacacagtccgagaacagctgcacccacttcccaggcaatctccccaacatgctgagagacctgagggacgccttctcccgcgtgaagacattcttccagatgaaggaccagctggacaatctcctgctgaaggagagcctgctggaggacttcaagggctacctgggctgccaggccctgtccgagatgatccagttctacctggaggaggtgatgccacaggccgagaaccaggaccccgacatcaaggcccacgtgaacagcctgggcgagaacctgaagaccctgcggctgagactgaggcgctgccaccggttcctgccatgcgagaacaagtccaaggccgtggaacaagtgaagaacgccttcaacaagctgcaggagaagggcatctacaaggccatgagcgagttcgacatcttcatcaactacatcgaggcctacatgacaatgaagatcagaaac

SEQ ID NO: 14 - RNA, 2124 nt, synthetic construct
  source 1..2124: mol_type = other RNA
  modified_base 2: mod_base = mo5u
  modified_base 4: mod_base = mo5u
  modified_base 8: mod_base = mo5u
  modified_base 10: mod_base = mo5u
  modified_base 20: mod_base = mo5u
  modified_base 23: mod_base = mo5u
  modified_base 26: mod_base = mo5u
  modified_base 29: mod_base = mo5u
  modified_base 32: mod_base = mo5u
  modified_base 35: mod_base = mo5u
  modified_base 38: mod_base = mo5u
  modified_base 41: mod_base = mo5u
  modified_base 46: mod_base = mo5u
  modified_base 49: mod_base = mo5u
  modified_base 52: mod_base = mo5u
  modified_base 53: mod_base = mo5u
  modified_base 83: mod_base = mo5u
  modified_base 86: mod_base = mo5u
  modified_base 89: mod_base = mo5u
  modified_base 91: mod_base = mo5u
  modified_base 92: mod_base = mo5u
  modified_base 102: mod_base = mo5u
  modified_base 104: mod_base = mo5u
  modified_base 116: mod_base = mo5u
  modified_base 131: mod_base = mo5u
  modified_base 142: mod_base = mo5u
  modified_base 146: mod_base = mo5u
  modified_base 148: mod_base = mo5u
  modified_base 154: mod_base = mo5u
  modified_base 160: mod_base = mo5u
  modified_base 170: mod_base = mo5u
  modified_base 179: mod_base = mo5u
  modified_base 190: mod_base = mo5u
  modified_base 200: mod_base = mo5u
  modified_base 212: mod_base = mo5u
  modified_base 215: mod_base = mo5u
  modified_base 218: mod_base = mo5u
  modified_base 221: mod_base = mo5u
  modified_base 233: mod_base = mo5u
  modified_base 235: mod_base = mo5u
  modified_base 239: mod_base = mo5u
  modified_base 257: mod_base = mo5u
  modified_base 272: mod_base = mo5u
  modified_base 281: mod_base = mo5u
  modified_base 295: mod_base = mo5u
  modified_base 302: mod_base = mo5u
  modified_base 311: mod_base = mo5u
  modified_base 319: mod_base = mo5u
  modified_base 320: mod_base = mo5u
  modified_base 328: mod_base = mo5u
  modified_base 329: mod_base = mo5u
  modified_base 341: mod_base = mo5u
  modified_base 346: mod_base = mo5u
  modified_base 362: mod_base = mo5u
  modified_base 367: mod_base = mo5u
  modified_base 370: mod_base = mo5u
  modified_base 374: mod_base = mo5u
  modified_base 382: mod_base = mo5u
  modified_base 385: mod_base = mo5u
  modified_base 393: mod_base = mo5u
  modified_base 395: mod_base = mo5u
  modified_base 407: mod_base = mo5u
  modified_base 410: mod_base = mo5u
  modified_base 421: mod_base = mo5u
  modified_base 422: mod_base = mo5u
  modified_base 433: mod_base = mo5u
  modified_base 434: mod_base = mo5u
  modified_base 440: mod_base = mo5u
  modified_base 442: mod_base = mo5u
  modified_base 452: mod_base = mo5u
  modified_base 461: mod_base = mo5u
  modified_base 466: mod_base = mo5u
  modified_base 467: mod_base = mo5u
  modified_base 476: mod_base = mo5u
  modified_base 478: mod_base = mo5u
  modified_base 481: mod_base = mo5u
  modified_base 497: mod_base = mo5u
  modified_base 503: mod_base = mo5u
  modified_base 508: mod_base = mo5u
  modified_base 509: mod_base = mo5u
  modified_base 515: mod_base = mo5u
  modified_base 535: mod_base = mo5u
  modified_base 541: mod_base = mo5u
  modified_base 542: mod_base = mo5u
  modified_base 560: mod_base = mo5u
  modified_base 563: mod_base = mo5u
  modified_base 574: mod_base = mo5u
  modified_base 575: mod_base = mo5u
  modified_base 593: mod_base = mo5u
  modified_base 610: mod_base = mo5u
  modified_base 611: mod_base = mo5u
  modified_base 626: mod_base = mo5u
  modified_base 628: mod_base = mo5u
  modified_base 635: mod_base = mo5u
  modified_base 647: mod_base = mo5u
  modified_base 650: mod_base = mo5u
  modified_base 653: mod_base = mo5u
  modified_base 664: mod_base = mo5u
  modified_base 665: mod_base = mo5u
  modified_base 688: mod_base = mo5u
  modified_base 694: mod_base = mo5u
  modified_base 695: mod_base = mo5u
  modified_base 700: mod_base = mo5u
  modified_base 701: mod_base = mo5u
  modified_base 704: mod_base = mo5u
  modified_base 707: mod_base = mo5u
  modified_base 708: mod_base = mo5u
  modified_base 718: mod_base = mo5u
  modified_base 721: mod_base = mo5u
  modified_base 730: mod_base = mo5u
  modified_base 748: mod_base = mo5u
  modified_base 758: mod_base = mo5u
  modified_base 763: mod_base = mo5u
  modified_base 766: mod_base = mo5u
  modified_base 784: mod_base = mo5u
  modified_base 802: mod_base = mo5u
  modified_base 803: mod_base = mo5u
  modified_base 808: mod_base = mo5u
  modified_base 809: mod_base = mo5u
  modified_base 815: mod_base = mo5u
  modified_base 817: mod_base = mo5u
  modified_base 827: mod_base = mo5u
  modified_base 829: mod_base = mo5u
  modified_base 862: mod_base = mo5u
  modified_base 868: mod_base = mo5u
  modified_base 869: mod_base = mo5u
  modified_base 874: mod_base = mo5u
  modified_base 875: mod_base = mo5u
  modified_base 878: mod_base = mo5u
  modified_base 880: mod_base = mo5u
  modified_base 881: mod_base = mo5u
  modified_base 895: mod_base = mo5u
  modified_base 898: mod_base = mo5u
  modified_base 904: mod_base = mo5u
  modified_base 931: mod_base = mo5u
  modified_base 937: mod_base = mo5u
  modified_base 940: mod_base = mo5u
  modified_base 958: mod_base = mo5u
  modified_base 974: mod_base = mo5u
  modified_base 976: mod_base = mo5u
  modified_base 979: mod_base = mo5u
  modified_base 982: mod_base = mo5u
  modified_base 983: mod_base = mo5u
  modified_base 985: mod_base = mo5u
  modified_base 1003: mod_base = mo5u
  modified_base 1010: mod_base = mo5u
  modified_base 1013: mod_base = mo5u
  modified_base 1037: mod_base = mo5u
  modified_base 1039: mod_base = mo5u
  modified_base 1043: mod_base = mo5u
  modified_base 1045: mod_base = mo5u
  modified_base 1046: mod_base = mo5u
  modified_base 1051: mod_base = mo5u
  modified_base 1052: mod_base = mo5u
  modified_base 1057: mod_base = mo5u
  modified_base 1058: mod_base = mo5u
  modified_base 1061: mod_base = mo5u
  modified_base 1072: mod_base = mo5u
  modified_base 1075: mod_base = mo5u
  modified_base 1081: mod_base = mo5u
  modified_base 1112: mod_base = mo5u
  modified_base 1114: mod_base = mo5u
  modified_base 1117: mod_base = mo5u
  modified_base 1129: mod_base = mo5u
  modified_base 1135: mod_base = mo5u
  modified_base 1136: mod_base = mo5u
  modified_base 1153: mod_base = mo5u
  modified_base 1159: mod_base = mo5u
  modified_base 1160: mod_base = mo5u
  modified_base 1162: mod_base = mo5u
  modified_base 1177: mod_base = mo5u
  modified_base 1180: mod_base = mo5u
  modified_base 1184: mod_base = mo5u
  modified_base 1184: mod_base = mo5u
  modified_base 1186: mod_base = mo5u
  modified_base 1187: mod_base = mo5u
  modified_base 1190: mod_base = mo5u
  modified_base 1193: mod_base = mo5u
  modified_base 1207: mod_base = mo5u
  modified_base 1208: mod_base = mo5u
  modified_base 1220: mod_base = mo5u
  modified_base 1226: mod_base = mo5u
  modified_base 1237: mod_base = mo5u
  modified_base 1241: mod_base = mo5u
  modified_base 1253: mod_base = mo5u
  modified_base 1256: mod_base = mo5u
  modified_base 1258: mod_base = mo5u
  modified_base 1265: mod_base = mo5u
  modified_base 1267: mod_base = mo5u
  modified_base 1273: mod_base = mo5u
  modified_base 1274: mod_base = mo5u
  modified_base 1292: mod_base = mo5u
  modified_base 1307: mod_base = mo5u
  modified_base 1316: mod_base = mo5u
  modified_base 1325: mod_base = mo5u
  modified_base 1327: mod_base = mo5u
  modified_base 1400: mod_base = mo5u
  modified_base 1402: mod_base = mo5u
  modified_base 1424: mod_base = mo5u
  modified_base 1468: mod_base = mo5u
  modified_base 1520: mod_base = mo5u
  modified_base 1526: mod_base = mo5u
  modified_base 1528: mod_base = mo5u
  modified_base 1535: mod_base = mo5u
  modified_base 1546: mod_base = mo5u
  modified_base 1553: mod_base = mo5u
  modified_base 1559: mod_base = mo5u
  modified_base 1561: mod_base = mo5u
  modified_base 1562: mod_base = mo5u
  modified_base 1571: mod_base = mo5u
  modified_base 1580: mod_base = mo5u
  modified_base 1592: mod_base = mo5u
  modified_base 1594: mod_base = mo5u
  modified_base 1598: mod_base = mo5u
  modified_base 1600: mod_base = mo5u
  modified_base 1601: mod_base = mo5u
  modified_base 1615: mod_base = mo5u
  modified_base 1618: mod_base = mo5u
  modified_base 1624: mod_base = mo5u
  modified_base 1625: mod_base = mo5u
  modified_base 1642: mod_base = mo5u
  modified_base 1660: mod_base = mo5u
  modified_base 1661: mod_base = mo5u
  modified_base 1664: mod_base = mo5u
  modified_base 1667: mod_base = mo5u
  modified_base 1678: mod_base = mo5u
  modified_base 1688: mod_base = mo5u
  modified_base 1700: mod_base = mo5u
  modified_base 1711: mod_base = mo5u
  modified_base 1712: mod_base = mo5u
  modified_base 1724: mod_base = mo5u
  modified_base 1726: mod_base = mo5u
  modified_base 1736: mod_base = mo5u
  modified_base 1738: mod_base = mo5u
  modified_base 1739: mod_base = mo5u
  modified_base 1741: mod_base = mo5u
  modified_base 1742: mod_base = mo5u
  modified_base 1744: mod_base = mo5u
  modified_base 1745: mod_base = mo5u
  modified_base 1760: mod_base = mo5u
  modified_base 1766: mod_base = mo5u
  modified_base 1768: mod_base = mo5u
  modified_base 1780: mod_base = mo5u
  modified_base 1784: mod_base = mo5u
  modified_base 1787: mod_base = mo5u
  modified_base 1802: mod_base = mo5u
  modified_base 1811: mod_base = mo5u
  modified_base 1817: mod_base = mo5u
  modified_base 1829: mod_base = mo5u
  modified_base 1838: mod_base = mo5u
  modified_base 1850: mod_base = mo5u
  modified_base 1871: mod_base = mo5u
  modified_base 1874: mod_base = mo5u
  modified_base 1879: mod_base = mo5u
  modified_base 1880: mod_base = mo5u
  modified_base 1882: mod_base = mo5u
  modified_base 1895: mod_base = mo5u
  modified_base 1897: mod_base = mo5u
  modified_base 1903: mod_base = mo5u
  modified_base 1904: mod_base = mo5u
  modified_base 1910: mod_base = mo5u
  modified_base 1922: mod_base = mo5u
  modified_base 1925: mod_base = mo5u
  modified_base 1942: mod_base = mo5u
  modified_base 1949: mod_base = mo5u
  modified_base 1958: mod_base = mo5u
  modified_base 1960: mod_base = mo5u
  modified_base 1961: mod_base = mo5u
  modified_base 1970: mod_base = mo5u
  modified_base 1976: mod_base = mo5u
  modified_base 1991: mod_base = mo5u
  modified_base 1993: mod_base = mo5u
  modified_base 1994: mod_base = mo5u
  modified_base 1999: mod_base = mo5u
  modified_base 2014: mod_base = mo5u
  modified_base 2017: mod_base = mo5u
  modified_base 2018: mod_base = mo5u
  modified_base 2020: mod_base = mo5u
  modified_base 2032: mod_base = mo5u
  modified_base 2033: mod_base = mo5u
  modified_base 2035: mod_base = mo5u
  modified_base 2038: mod_base = mo5u
  modified_base 2045: mod_base = mo5u
  modified_base 2050: mod_base = mo5u
  modified_base 2063: mod_base = mo5u
  modified_base 2072: mod_base = mo5u
  modified_base 2081: mod_base = mo5u
  modified_base 2085: mod_base = mo5u
  modified_base 2090: mod_base = mo5u
  modified_base 2095: mod_base = mo5u
  modified_base 2102: mod_base = mo5u
  modified_base 2105: mod_base = mo5u
  modified_base 2110: mod_base = mo5u
  modified_base 2122: mod_base = mo5u
atgtccctgtggcagcccctggtgctggtgctgctggtgctgggctgctgcttcgccgccccaaggcagcgccagagcacactggtgctgttccccggcgatctccggaccaacctgacagacagacagctggccgaggagtacctgtacaggtacggctacacccgcgtggccgagatgcggggcgagtccaagagcctgggcccagccctgctgctgctccagaagcagctgtccctgcccgagacaggcgagctggacagcgccaccctgaaggccatgagaacaccaaggtgcggcgtgcccgacctgggccgcttccagaccttcgagggcgacctgaagtggcaccaccacaacatcacatactggatccagaactactccgaggatctcccacgggccgtgatcgacgacgccttcgccagagccttcgccctgtggagcgccgtgacccccctgacattcaccagggtgtactcccgcgacgccgacatcgtgatccagttcggcgtggccgagcacggcgacggctacccattcgacggcaaggacggcctgctggcccacgccttccccccaggccccggcatccagggcgacgcccacttcgacgacgacgagctgtggagcctgggcaagggcgtggtggtgccaacacggttcggcaacgccgacggcgccgcctgccacttccccttcatcttcgagggcagatcctacagcgcctgcaccacagacgggcggtccgacggcctgccatggtgcagcaccacagccaactacgacaccgacgaccgcttcggcttctgcccctccgagcggctgtacacacaggacggcaacgccgacggcaagccatgccagttccccttcatcttccagggccagagctactccgcctgcaccacagacggcagaagcgacggctacaggtggtgcgccaccacagccaactacgaccgcgacaagctgttcggcttctgcccaacccgggccgactccacagtgatgggcggcaacagcgccggcgagctgtgcgtgttccccttcaccttcctgggcaaggagtactccacatgcaccagcgagggcagaggcgacggcaggctgtggtgcgccacaacctccaacttcgacagcgacaagaagtggggcttctgcccagaccagggctactccctgttcctggtggccgcccacgagttcggccacgccctgggcctggaccacagctccgtgcccgaggccctgatgtacccaatgtaccgcttcacagagggccccccactgcacaaggacgacgtgaacggcatccggcacctgtacggccccagaccagagcccgagccaaggcccccaaccacaaccacaccccagccaaccgcccccccaacagtgtgccccaccggcccacccacagtgcacccaagcgagcgccccaccgccggcccaacaggccccccatccgccggccccaccggcccacccacagccggcccaagcaccgccaccaccgtgcccctgtccccagtggacgacgcctgcaacgtgaacatcttcgacgccatcgccgagatcggcaaccagctgtacctgttcaaggacggcaagtactggcggttcagcgagggcagaggctccaggccccagggcccattcctgatcgccgacaagtggcccgccctgccacgcaagctggacagcgtgttcgaggagcggctgtccaagaagctgttcttcttcagcggcagacaggtgtgggtgtacaccggcgcctccgtgctgggccccaggcgcctggacaagctgggcctgggcgccgacgtggcccaggtgaccggcgccctgcggagcggcagaggcaagatgctgctgttctccggcaggcgcctgtggcggttcgacgtgaaggcccagatggtggaccccagaagcgcctccgaggtggacaggatgttccccggcgtgcccctggacacccacgacgtgttccagtaccgcgagaaggcctacttctgccaggaccggttctactggagagtgagctccaggagcgagctgaaccaggtggaccaggtgggctacgtgacctacgacatcctgcagtgccccgaggactaa

SEQ ID NO: 15 - RNA, 66 nt, synthetic construct
  source 1..66: mol_type = other RNA
  modified_base 4: mod_base = mo5u
  modified_base 19: mod_base = mo5u
  modified_base 20: mod_base = mo5u
  modified_base 26: mod_base = mo5u
  modified_base 29: mod_base = mo5u
  modified_base 47: mod_base = mo5u
ggctccggcgccaccaacttcagcctgctgaagcaggccggcgacgtggaggagaaccccggccca

SEQ ID NO: 16 - RNA, 3111 nt, synthetic construct
  source 1..3111: mol_type = other RNA
  modified_base 12: mod_base = mo5u
  modified_base 13: mod_base = mo5u
  modified_base 14: mod_base = mo5u
  modified_base 16: mod_base = mo5u
  modified_base 21: mod_base = mo5u
  modified_base 30: mod_base = mo5u
  modified_base 31: mod_base = mo5u
  modified_base 32: mod_base = mo5u
  modified_base 34: mod_base = mo5u
  modified_base 39: mod_base = mo5u
  modified_base 48: mod_base = mo5u
  modified_base 49: mod_base = mo5u
  modified_base 50: mod_base = mo5u
  modified_base 52: mod_base = mo5u
  modified_base 57: mod_base = mo5u
  modified_base 65: mod_base = mo5u
  modified_base 66: mod_base = mo5u
  modified_base 67: mod_base = mo5u
  modified_base 68: mod_base = mo5u
  modified_base 70: mod_base = mo5u
  modified_base 75: mod_base = mo5u
  modified_base 84: mod_base = mo5u
  modified_base 85: mod_base = mo5u
  modified_base 86: mod_base = mo5u
  modified_base 88: mod_base = mo5u
  modified_base 93: mod_base = mo5u
  modified_base 102: mod_base = mo5u
  modified_base 103: mod_base = mo5u
  modified_base 104: mod_base = mo5u
  modified_base 106: mod_base = mo5u
  modified_base 111: mod_base = mo5u
  modified_base 120: mod_base = mo5u
  modified_base 121: mod_base = mo5u
  modified_base 122: mod_base = mo5u
  modified_base 124: mod_base = mo5u
  modified_base 129: mod_base = mo5u
  modified_base 138: mod_base = mo5u
  modified_base 139: mod_base = mo5u
  modified_base 140: mod_base = mo5u
  modified_base 142: mod_base = mo5u
  modified_base 147: mod_base = mo5u
  modified_base 156: mod_base = mo5u
  modified_base 157: mod_base = mo5u
  modified_base 158: mod_base = mo5u
  modified_base 160: mod_base = mo5u
  modified_base 174: mod_base = mo5u
  modified_base 175: mod_base = mo5u
  modified_base 176: mod_base = mo5u
  modified_base 178: mod_base = mo5u
  modified_base 183: mod_base = mo5u
  modified_base 184: mod_base = mo5u
  modified_base 197: mod_base = mo5u
  modified_base 207: mod_base = mo5u
  modified_base 215: mod_base = mo5u
  modified_base 222: mod_base = mo5u
  modified_base 227: mod_base = mo5u
  modified_base 230: mod_base = mo5u
  modified_base 234: mod_base = mo5u
  modified_base 237: mod_base = mo5u
  modified_base 240: mod_base = mo5u
  modified_base 243: mod_base = mo5u
  modified_base 252: mod_base = mo5u
  modified_base 281: mod_base = mo5u
  modified_base 293: mod_base = mo5u
  modified_base 302: mod_base = mo5u
  modified_base 303: mod_base = mo5u
  modified_base 313: mod_base = mo5u
  modified_base 315: mod_base = mo5u
  modified_base 324: mod_base = mo5u
  modified_base 327: mod_base = mo5u
  modified_base 336: mod_base = mo5u
  modified_base 347: mod_base = mo5u
  modified_base 348: mod_base = mo5u
  modified_base 350: mod_base = mo5u
  modified_base 365: mod_base = mo5u
  modified_base 366: mod_base = mo5u
  modified_base 368: mod_base = mo5u
  modified_base 369: mod_base = mo5u
  modified_base 375: mod_base = mo5u
  modified_base 387: mod_base = mo5u
  modified_base 394: mod_base = mo5u
  modified_base 399: mod_base = mo5u
  modified_base 402: mod_base = mo5u
  modified_base 414: mod_base = mo5u
  modified_base 417: mod_base = mo5u
  modified_base 425: mod_base = mo5u
  modified_base 426: mod_base = mo5u
  modified_base 434: mod_base = mo5u
  modified_base 438: mod_base = mo5u
  modified_base 443: mod_base = mo5u
  modified_base 453: mod_base = mo5u
  modified_base 455: mod_base = mo5u
  modified_base 462: mod_base = mo5u
  modified_base 465: mod_base = mo5u
  modified_base 470: mod_base = mo5u
  modified_base 471: mod_base = mo5u
  modified_base 473: mod_base = mo5u
  modified_base 477: mod_base = mo5u
  modified_base 486: mod_base = mo5u
  modified_base 489: mod_base = mo5u
  modified_base 519: mod_base = mo5u
  modified_base 531: mod_base = mo5u
  modified_base 540: mod_base = mo5u
  modified_base 552: mod_base = mo5u
  modified_base 561: mod_base = mo5u
  modified_base 567: mod_base = mo5u
  modified_base 573: mod_base = mo5u
  modified_base 581: mod_base = mo5u
  modified_base 590: mod_base = mo5u
  modified_base 591: mod_base = mo5u
  modified_base 594: mod_base = mo5u
  modified_base 598: mod_base = mo5u
  modified_base 611: mod_base = mo5u
  modified_base 621: mod_base = mo5u
  modified_base 630: mod_base = mo5u
  modified_base 641: mod_base = mo5u
  modified_base 642: mod_base = mo5u
  modified_base 651: mod_base = mo5u
  modified_base 666: mod_base = mo5u
  modified_base 668: mod_base = mo5u
  modified_base 678: mod_base = mo5u
  modified_base 686: mod_base = mo5u
  modified_base 687: mod_base = mo5u
  modified_base 693: mod_base = mo5u
  modified_base 695: mod_base = mo5u
  modified_base 696: mod_base = mo5u
  modified_base 699: mod_base = mo5u
  modified_base 704: mod_base = mo5u
  modified_base 708: mod_base = mo5u
  modified_base 716: mod_base = mo5u
  modified_base 720: mod_base = mo5u
  modified_base 726: mod_base = mo5u
  modified_base 732: mod_base = mo5u
  modified_base 743: mod_base = mo5u
  modified_base 758: mod_base = mo5u
  modified_base 759: mod_base = mo5u
  modified_base 765: mod_base = mo5u
  modified_base 768: mod_base = mo5u
  modified_base 786: mod_base = mo5u
  modified_base 807: mod_base = mo5u
  modified_base 809: mod_base = mo5u
  modified_base 813: mod_base = mo5u
  modified_base 815: mod_base = mo5u
  modified_base 825: mod_base = mo5u
  modified_base 828: mod_base = mo5u
  modified_base 831: mod_base = mo5u
  modified_base 834: mod_base = mo5u
  modified_base 837: mod_base = mo5u
  modified_base 840: mod_base = mo5u
  modified_base 843: mod_base = mo5u
  modified_base 846: mod_base = mo5u
  modified_base 851: mod_base = mo5u
  modified_base 854: mod_base = mo5u
  modified_base 857: mod_base = mo5u
  modified_base 858: mod_base = mo5u
  modified_base 888: mod_base = mo5u
  modified_base 891: mod_base = mo5u
  modified_base 894: mod_base = mo5u
  modified_base 896: mod_base = mo5u
  modified_base 897: mod_base = mo5u
  modified_base 907: mod_base = mo5u
  modified_base 909: mod_base = mo5u
  modified_base 921: mod_base = mo5u
  modified_base 936: mod_base = mo5u
  modified_base 947: mod_base = mo5u
  modified_base 951: mod_base = mo5u
  modified_base 953: mod_base = mo5u
  modified_base 959: mod_base = mo5u
  modified_base 965: mod_base = mo5u
  modified_base 975: mod_base = mo5u
  modified_base 984: mod_base = mo5u
  modified_base 995: mod_base = mo5u
  modified_base 1005: mod_base = mo5u
  modified_base 1017: mod_base = mo5u
  modified_base 1020: mod_base = mo5u
  modified_base 1023: mod_base = mo5u
  modified_base 1026: mod_base = mo5u
  modified_base 1038: mod_base = mo5u
  modified_base 1040: mod_base = mo5u
  modified_base 1044: mod_base = mo5u
  modified_base 1062: mod_base = mo5u
  modified_base 1077: mod_base = mo5u
  modified_base 1083: mod_base = mo5u
  modified_base 1086: mod_base = mo5u
  modified_base 1098: mod_base = mo5u
  modified_base 1100: mod_base = mo5u
  modified_base 1107: mod_base = mo5u
  modified_base 1116: mod_base = mo5u
  modified_base 1124: mod_base = mo5u
  modified_base 1125: mod_base = mo5u
  modified_base 1134: mod_base = mo5u
  modified_base 1135: mod_base = mo5u
  modified_base 1146: mod_base = mo5u
  modified_base 1151: mod_base = mo5u
  modified_base 1167: mod_base = mo5u
  modified_base 1172: mod_base = mo5u
  modified_base 1175: mod_base = mo5u
  modified_base 1179: mod_base = mo5u
  modified_base 1187: mod_base = mo5u
  modified_base 1190: mod_base = mo5u
  modified_base 1198: mod_base = mo5u
  modified_base 1200: mod_base = mo5u
  modified_base 1212: mod_base = mo5u
  modified_base 1215: mod_base = mo5u
  modified_base 1226: mod_base = mo5u
  modified_base 1271: mod_base = mo5u
  modified_base 1272: mod_base = mo5u
  modified_base 1281: mod_base = mo5u
  modified_base 1283: mod_base = mo5u
  modified_base 1286: mod_base = mo5u
  modified_base 1302: mod_base = mo5u
  modified_base 1305: mod_base = mo5u
  modified_base 1308: mod_base = mo5u
  modified_base 1313: mod_base = mo5u
  modified_base 1314: mod_base = mo5u
  modified_base 1320: mod_base = mo5u
  modified_base 1340: mod_base = mo5u
  modified_base 1346: mod_base = mo5u
  modified_base 1347: mod_base = mo5u
  modified_base 1365: mod_base = mo5u
  modified_base 1368: mod_base = mo5u
  modified_base 1379: mod_base = mo5u
  modified_base 1380: mod_base = mo5u
  modified_base 1398: mod_base = mo5u
  modified_base 1415: mod_base = mo5u
  modified_base 1416: mod_base = mo5u
  modified_base 1431: mod_base = mo5u
  modified_base 1433: mod_base = mo5u
  modified_base 1440: mod_base = mo5u
  modified_base 1452: mod_base = mo5u
  modified_base 1455: mod_base = mo5u
  modified_base 1458: mod_base = mo5u
  modified_base 1469: mod_base = mo5u
  modified_base 1470: mod_base = mo5u
  modified_base 1493: mod_base = mo5u
  modified_base 1499: mod_base = mo5u
  modified_base 1500: mod_base = mo5u
  modified_base 1505: mod_base = mo5u
  modified_base 1506: mod_base = mo5u
  modified_base 1509: mod_base = mo5u
  modified_base 1511: mod_base = mo5u
  modified_base 1512: mod_base = mo5u
  modified_base 1523: mod_base = mo5u
  modified_base 1526: mod_base = mo5u
  modified_base 1535: mod_base = mo5u
  modified_base 1553: mod_base = mo5u
  modified_base 1563: mod_base = mo5u
  modified_base 1568: mod_base = mo5u
  modified_base 1571: mod_base = mo5u
  modified_base 1589: mod_base = mo5u
  modified_base 1607: mod_base = mo5u
  modified_base 1608: mod_base = mo5u
  modified_base 1613: mod_base = mo5u
  modified_base 1614: mod_base = mo5u
  modified_base 1616: mod_base = mo5u
  modified_base 1632: mod_base = mo5u
  modified_base 1634: mod_base = mo5u
  modified_base 1667: mod_base = mo5u
  modified_base 1673: mod_base = mo5u
  modified_base 1674: mod_base = mo5u
  modified_base 1679: mod_base = mo5u
  modified_base 1680: mod_base = mo5u
  modified_base 1683: mod_base = mo5u
  modified_base 1685: mod_base = mo5u
  modified_base 1686: mod_base = mo5u
  modified_base 1700: mod_base = mo5u
  modified_base 1703: mod_base = mo5u
  modified_base 1709: mod_base = mo5u
  modified_base 1736: mod_base = mo5u
  modified_base 1313: mod_base = mo5u
  modified_base 1314: mod_base = mo5u
  modified_base 1320: mod_base = mo5u
  modified_base 1340: mod_base = mo5u
  modified_base 1346: mod_base = mo5u
  modified_base 1347: mod_base = mo5u
  modified_base 1365: mod_base = mo5u
  modified_base 1368: mod_base = mo5u
  modified_base 1379: mod_base = mo5u
  modified_base 1380: mod_base = mo5u
  modified_base 1398: mod_base = mo5u
  modified_base 1415: mod_base = mo5u
  modified_base 1416: mod_base = mo5u
  modified_base 1431: mod_base = mo5u
  modified_base 1433: mod_base = mo5u
  modified_base 1440: mod_base = mo5u
  modified_base 1452: mod_base = mo5u
  modified_base 1455: mod_base = mo5u
  modified_base 1458: mod_base = mo5u
  modified_base 1469: mod_base = mo5u
  modified_base 1470: mod_base = mo5u
  modified_base 1493: mod_base = mo5u
  modified_base 1499: mod_base = mo5u
  modified_base 1500: mod_base = mo5u
  modified_base 1505: mod_base = mo5u
  modified_base 1506: mod_base = mo5u
  modified_base 1509: mod_base = mo5u
  modified_base 1511: mod_base = mo5u
  modified_base 1512: mod_base = mo5u
  modified_base 1523: mod_base = mo5u
  modified_base 1526: mod_base = mo5u
  modified_base 1535: mod_base = mo5u
  modified_base 1553: mod_base = mo5u
  modified_base 1563: mod_base = mo5u
  modified_base 1568: mod_base = mo5u
  modified_base 1571: mod_base = mo5u
  modified_base 1589: mod_base = mo5u
  modified_base 1607: mod_base = mo5u
  modified_base 1608: mod_base = mo5u
  modified_base 1613: mod_base = mo5u
  modified_base 1614: mod_base = mo5u
  modified_base 1616: mod_base = mo5u
  modified_base 1632: mod_base = mo5u
  modified_base 1634: mod_base = mo5u
  modified_base 1667: mod_base = mo5u
  modified_base 1673: mod_base = mo5u
  modified_base 1674: mod_base = mo5u
  modified_base 1679: mod_base = mo5u
  modified_base 1680: mod_base = mo5u
  modified_base 1683: mod_base = mo5u
  modified_base 1685: mod_base = mo5u
  modified_base 1686: mod_base = mo5u
  modified_base 1700: mod_base = mo5u
  modified_base 1703: mod_base = mo5u
  modified_base 1709: mod_base = mo5u
  modified_base 1736: mod_base = mo5u
  modified_base 1742: mod_base = mo5u
  modified_base 1745: mod_base = mo5u
  modified_base 1763: mod_base = mo5u
  modified_base 1779: mod_base = mo5u
  modified_base 1781: mod_base = mo5u
  modified_base 1782: mod_base = mo5u
  modified_base 1787: mod_base = mo5u
  modified_base 1788: mod_base = mo5u
  modified_base 1790: mod_base = mo5u
  modified_base 1808: mod_base = mo5u
  modified_base 1815: mod_base = mo5u
  modified_base 1818: mod_base = mo5u
  modified_base 1842: mod_base = mo5u
  modified_base 1844: mod_base = mo5u
  modified_base 1848: mod_base = mo5u
  modified_base 1850: mod_base = mo5u
  modified_base 1851: mod_base = mo5u
  modified_base 1857: mod_base = mo5u
  modified_base 1858: mod_base = mo5u
  modified_base 1862: mod_base = mo5u
  modified_base 1863: mod_base = mo5u
  modified_base 1866: mod_base = mo5u
  modified_base 1877: mod_base = mo5u
  modified_base 1880: mod_base = mo5u
  modified_base 1886: mod_base = mo5u
  modified_base 1917: mod_base = mo5u
  modified_base 1919: mod_base = mo5u
  modified_base 1922: mod_base = mo5u
  modified_base 1934: mod_base = mo5u
  modified_base 1940: mod_base = mo5u
  modified_base 1941: mod_base = mo5u
  modified_base 1958: mod_base = mo5u
  modified_base 1964: mod_base = mo5u
  modified_base 1965: mod_base = mo5u
  modified_base 1967: mod_base = mo5u
  modified_base 1982: mod_base = mo5u
  modified_base 1985: mod_base = mo5u
  modified_base 1989: mod_base = mo5u
  modified_base 1991: mod_base = mo5u
  modified_base 1992: mod_base = mo5u
  modified_base 1995: mod_base = mo5u
  modified_base 1998: mod_base = mo5u
  modified_base 2012: mod_base = mo5u
  modified_base 2013: mod_base = mo5u
  modified_base 2025: mod_base = mo5u
  modified_base 2031: mod_base = mo5u
  modified_base 2042: mod_base = mo5u
  modified_base 2046: mod_base = mo5u
  modified_base 2058: mod_base = mo5u
  modified_base 2061: mod_base = mo5u
  modified_base 2063: mod_base = mo5u
  modified_base 2070: mod_base = mo5u
  modified_base 2072: mod_base = mo5u
  modified_base 2078: mod_base = mo5u
  modified_base 2079: mod_base = mo5u
  modified_base 2097: mod_base = mo5u
  modified_base 2112: mod_base = mo5u
  modified_base 2121: mod_base = mo5u
  modified_base 2130: mod_base = mo5u
  modified_base 2132: mod_base = mo5u
  modified_base 2205: mod_base = mo5u
  modified_base 2207: mod_base = mo5u
  modified_base 2229: mod_base = mo5u
  modified_base 2273: mod_base = mo5u
  modified_base 2273: mod_base = mo5u
  modified_base 2325: mod_base = mo5u
  modified_base 2331: mod_base = mo5u
  modified_base 2333: mod_base = mo5u
  modified_base 2340: mod_base = mo5u
  modified_base 2351: mod_base = mo5u
  modified_base 2358: mod_base = mo5u
  modified_base 2364: mod_base = mo5u
  modified_base 2366: mod_base = mo5u
  modified_base 2367: mod_base = mo5u
  modified_base 2376: mod_base = mo5u
  modified_base 2385: mod_base = mo5u
  modified_base 2397: mod_base = mo5u
  modified_base 2399: mod_base = mo5u
  modified_base 2403: mod_base = mo5u
  modified_base 2405: mod_base = mo5u
  modified_base 2406: mod_base = mo5u
  modified_base 2419: mod_base = mo5u
  modified_base 2423: mod_base = mo5u
  modified_base 2429: mod_base = mo5u
  modified_base 2430: mod_base = mo5u
  modified_base 2447: mod_base = mo5u
  modified_base 2465: mod_base = mo5u
  modified_base 2466: mod_base = mo5u
  modified_base 2469: mod_base = mo5u
  modified_base 2472: mod_base = mo5u
  modified_base 2483: mod_base = mo5u
  modified_base 2493: mod_base = mo5u
  modified_base 2505: mod_base = mo5u
  modified_base 2514: mod_base = mo5u
  modified_base 2516: mod_base = mo5u
  modified_base 2517: mod_base = mo5u
  modified_base 2529: mod_base = mo5u
  modified_base 2531: mod_base = mo5u
  modified_base 2541: mod_base = mo5u
  modified_base 2543: mod_base = mo5u
  modified_base 2544: mod_base = mo5u
  modified_base 2546: mod_base = mo5u
  modified_base 2547: mod_base = mo5u
  modified_base 2549: mod_base = mo5u
  modified_base 2550: mod_base = mo5u
  modified_base 2565: mod_base = mo5u
  modified_base 2567: mod_base = mo5u
  modified_base 2571: mod_base = mo5u
  modified_base 2573: mod_base = mo5u
  modified_base 2585: mod_base = mo5u
  modified_base 2589: mod_base = mo5u
  modified_base 2592: mod_base = mo5u
  modified_base 2607: mod_base = mo5u
  modified_base 2616: mod_base = mo5u
  modified_base 2622: mod_base = mo5u
  modified_base 2634: mod_base = mo5u
  modified_base 2643: mod_base = mo5u
  modified_base 2655: mod_base = mo5u
  modified_base 2676: mod_base = mo5u
  modified_base 2679: mod_base = mo5u
  modified_base 2682: mod_base = mo5u
  modified_base 2824: mod_base = mo5u
  modified_base 2825: mod_base = mo5u
  modified_base 2827: mod_base = mo5u
  modified_base 2700: mod_base = mo5u
  modified_base 2702: mod_base = mo5u
  modified_base 2708: mod_base = mo5u
  modified_base 2709: mod_base = mo5u
  modified_base 2715: mod_base = mo5u
  modified_base 2727: mod_base = mo5u
  modified_base 2730: mod_base = mo5u
  modified_base 2747: mod_base = mo5u
  modified_base 2754: mod_base = mo5u
  modified_base 2763: mod_base = mo5u
  modified_base 2765: mod_base = mo5u
  modified_base 2766: mod_base = mo5u
  modified_base 2775: mod_base = mo5u
  modified_base 2781: mod_base = mo5u
  modified_base 2796: mod_base = mo5u
  modified_base 2798: mod_base = mo5u
  modified_base 2799: mod_base = mo5u
  modified_base 2804: mod_base = mo5u
  modified_base 2819: mod_base = mo5u
  modified_base 2822: mod_base = mo5u
  modified_base 2823: mod_base = mo5u
  modified_base 2825: mod_base = mo5u
  modified_base 2837: mod_base = mo5u
  modified_base 2838: mod_base = mo5u
  modified_base 2840: mod_base = mo5u
  modified_base 2843: mod_base = mo5u
  modified_base 2850: mod_base = mo5u
  modified_base 2855: mod_base = mo5u
  modified_base 2868: mod_base = mo5u
  modified_base 2877: mod_base = mo5u
  modified_base 2886: mod_base = mo5u
  modified_base 2891: mod_base = mo5u
  modified_base 2895: mod_base = mo5u
  modified_base 2900: mod_base = mo5u
  modified_base 2907: mod_base = mo5u
  modified_base 2910: mod_base = mo5u
  modified_base 2915: mod_base = mo5u
  modified_base 2927: mod_base = mo5u
  modified_base 2932: mod_base = mo5u
  modified_base 2937: mod_base = mo5u
  modified_base 2939: mod_base = mo5u
  modified_base 2840: mod_base = mo5u
  modified_base 2941: mod_base = mo5u
  modified_base 2942: mod_base = mo5u
  modified_base 2946: mod_base = mo5u
  modified_base 2950: mod_base = mo5u
  modified_base 2951: mod_base = mo5u
  modified_base 2952: mod_base = mo5u
  modified_base 2953: mod_base = mo5u
  modified_base 2954: mod_base = mo5u
  modified_base 2957: mod_base = mo5u
  modified_base 2958: mod_base = mo5u
  modified_base 2960: mod_base = mo5u
  modified_base 2961: mod_base = mo5u
  modified_base 2975: mod_base = mo5u
  modified_base 2980: mod_base = mo5u
  modified_base 2996: mod_base = mo5u
  modified_base 3000: mod_base = mo5u
  modified_base 3002: mod_base = mo5u
  modified_base 3008: mod_base = mo5u
  modified_base 3010: mod_base = mo5u
  modified_base 3012: mod_base = mo5u
  modified_base 3014: mod_base = mo5u
  modified_base 3015: mod_base = mo5u
  modified_base 3017: mod_base = mo5u
aggccggcgggtttctgacatccggcgggtttctgacatccggcgggtttctgacatccggcgggtttctgacatccggcgggtttctgacatccggcgggtttctgacatccggcgggtttctgacatccggcgggtttctgacatccggcgggtttctgacatccggcgggtttctgacattcacaaccaggcctccacaaccatgcacagctccgccctgctgtgctgcctggtgctgctgaccggcgtgcgggccagccccggccagggcacacagtccgagaacagctgcacccacttcccaggcaatctccccaacatgctgagagacctgagggacgccttctcccgcgtgaagacattcttccagatgaaggaccagctggacaatctcctgctgaaggagagcctgctggaggacttcaagggctacctgggctgccaggccctgtccgagatgatccagttctacctggaggaggtgatgccacaggccgagaaccaggaccccgacatcaaggcccacgtgaacagcctgggcgagaacctgaagaccctgcggctgagactgaggcgctgccaccggttcctgccatgcgagaacaagtccaaggccgtggaacaagtgaagaacgccttcaacaagctgcaggagaagggcatctacaaggccatgagcgagttcgacatcttcatcaactacatcgaggcctacatgacaatgaagatcagaaacggctccggcgccaccaacttcagcctgctgaagcaggccggcgacgtggaggagaaccccggcccaatgtccctgtggcagcccctggtgctggtgctgctggtgctgggctgctgcttcgccgccccaaggcagcgccagagcacactggtgctgttccccggcgatctccggaccaacctgacagacagacagctggccgaggagtacctgtacaggtacggctacacccgcgtggccgagatgcggggcgagtccaagagcctgggcccagccctgctgctgctccagaagcagctgtccctgcccgagacaggcgagctggacagcgccaccctgaaggccatgagaacaccaaggtgcggcgtgcccgacctgggccgcttccagaccttcgagggcgacctgaagtggcaccaccacaacatcacatactggatccagaactactccgaggatctcccacgggccgtgatcgacgacgccttcgccagagccttcgccctgtggagcgccgtgacccccctgacattcaccagggtgtactcccgcgacgccgacatcgtgatccagttcggcgtggccgagcacggcgacggctacccattcgacggcaaggacggcctgctggcccacgccttccccccaggccccggcatccagggcgacgcccacttcgacgacgacgagctgtggagcctgggcaagggcgtggtggtgccaacacggttcggcaacgccgacggcgccgcctgccacttccccttcatcttcgagggcagatcctacagcgcctgcaccacagacgggcggtccgacggcctgccatggtgcagcaccacagccaactacgacaccgacgaccgcttcggcttctgcccctccgagcggctgtacacacaggacggcaacgccgacggcaagccatgccagttccccttcatcttccagggccagagctactccgcctgcaccacagacggcagaagcgacggctacaggtggtgcgccaccacagccaactacgaccgcgacaagctgttcggcttctgcccaacccgggccgactccacagtgatgggcggcaacagcgccggcgagctgtgcgtgttccccttcaccttcctgggcaaggagtactccacatgcaccagcgagggcagaggcgacggcaggctgtggtgcgccacaacctccaacttcgacagcgacaagaagtggggcttctgcccagaccagggctactccctgttcctggtggccgcccacgagttcggccacgccctgggcctggaccacagctccgtgcccgaggccctgatgtacccaatgtaccgcttcacagagggccccccactgcacaaggacgacgtgaacggcatccggcacctgtacggccccagaccagagcccgagccaaggcccccaaccacaaccacaccccagccaaccgcccccccaacagtgtgccccaccggcccacccacagtgcacccaagcgagcgccccaccgccggcccaacaggccccccatccgccggccccaccggcccacccacagccggcccaagcaccgccaccaccgtgcccctgtccccagtggacgacgcctgcaacgtgaacatcttcgacgccatcgccgagatcggcaaccagctgtacctgttcaaggacggcaagtactggcggttcagcgagggcagaggctccaggccccagggcccattcctgatcgccgacaagtggcccgccctgccacgcaagctggacagcgtgttcgaggagcggctgtccaagaagctgttcttcttcagcggcagacaggtgtgggtgtacaccggcgcctccgtgctgggccccaggcgcctggacaagctgggcctgggcgccgacgtggcccaggtgaccggcgccctgcggagcggcagaggcaagatgctgctgttctccggcaggcgcctgtggcggttcgacgtgaaggcccagatggtggaccccagaagcgcctccgaggtggacaggatgttccccggcgtgcccctggacacccacgacgtgttccagtaccgcgagaaggcctacttctgccaggaccggttctactggagagtgagctccaggagcgagctgaaccaggtggaccaggtgggctacgtgacctacgacatcctgcagtgccccgaggactaaactcgagtgttttggctgggtttttccttgttcgcaccggacacctccagtgaccagacggcaaggtttttatcccagtgtatattgtcgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 205 nt, synthetic construct
  source 1..205: mol_type = other RNA
  modified_base 12: mod_base = mo5u
  modified_base 13: mod_base = mo5u
  modified_base 14: mod_base = mo5u
  modified_base 16: mod_base = mo5u
  modified_base 21: mod_base = mo5u
  modified_base 30: mod_base = mo5u
  modified_base 32: mod_base = mo5u
  modified_base 34: mod_base = mo5u
  modified_base 39: mod_base = mo5u
  modified_base 48: mod_base = mo5u
  modified_base 49: mod_base = mo5u
  modified_base 50: mod_base = mo5u
  modified_base 52: mod_base = mo5u
  modified_base 57: mod_base = mo5u
  modified_base 66: mod_base = mo5u
  modified_base 67: mod_base = mo5u
  modified_base 68: mod_base = mo5u
  modified_base 70: mod_base = mo5u
  modified_base 75: mod_base = mo5u
  modified_base 84: mod_base = mo5u
  modified_base 85: mod_base = mo5u
  modified_base 86: mod_base = mo5u
  modified_base 88: mod_base = mo5u
  modified_base 93: mod_base = mo5u
  modified_base 102: mod_base = mo5u
  modified_base 103: mod_base = mo5u
  modified_base 104: mod_base = mo5u
  modified_base 106: mod_base = mo5u
  modified_base 111: mod_base = mo5u
  modified_base 120: mod_base = mo5u
  modified_base 122: mod_base = mo5u
  modified_base 124: mod_base = mo5u
  modified_base 129: mod_base = mo5u
  modified_base 138: mod_base = mo5u
  modified_base 139: mod_base = mo5u
  modified_base 140: mod_base = mo5u
  modified_base 142: mod_base = mo5u
  modified_base 147: mod_base = mo5u
  modified_base 156: mod_base = mo5u
  modified_base 157: mod_base = mo5u
  modified_base 158: mod_base = mo5u
  modified_base 160: mod_base = mo5u
  modified_base 165: mod_base = mo5u
  modified_base 174: mod_base = mo5u
  modified_base 175: mod_base = mo5u
  modified_base 176: mod_base = mo5u
  modified_base 178: mod_base = mo5u
  modified_base 183: mod_base = mo5u
  modified_base 184: mod_base = mo5u
  modified_base 197: mod_base = mo5u
aggccggcgggtttctgacatccggcgggtttctgacatccggcgggtttctgacatccggcgggtttctgacatccggcgggtttctgacatccggcgggtttctgacatccggcgggtttctgacatccggcgggtttctgacatccggcgggtttctgacatccggcgggtttctgacattcacaaccaggcctccacaacc

SEQ ID NO: 18 - RNA, 92 nt, synthetic construct
  source 1..92: mol_type = other RNA
  modified_base 3: mod_base = mo5u
  modified_base 8: mod_base = mo5u
  modified_base 10: mod_base = mo5u
  modified_base 11: mod_base = mo5u
  modified_base 12: mod_base = mo5u
  modified_base 17: mod_base = mo5u
  modified_base 21: mod_base = mo5u
  modified_base 22: mod_base = mo5u
  modified_base 23: mod_base = mo5u
  modified_base 24: mod_base = mo5u
  modified_base 25: mod_base = mo5u
  modified_base 28: mod_base = mo5u
  modified_base 29: mod_base = mo5u
  modified_base 31: mod_base = mo5u
  modified_base 32: mod_base = mo5u
  modified_base 46: mod_base = mo5u
  modified_base 51: mod_base = mo5u
  modified_base 67: mod_base = mo5u
  modified_base 68: mod_base = mo5u
  modified_base 69: mod_base = mo5u
  modified_base 70: mod_base = mo5u
  modified_base 71: mod_base = mo5u
  modified_base 73: mod_base = mo5u
  modified_base 79: mod_base = mo5u
  modified_base 81: mod_base = mo5u
  modified_base 83: mod_base = mo5u
  modified_base 85: mod_base = mo5u
  modified_base 86: mod_base = mo5u
  modified_base 88: mod_base = mo5u
actcgagtgttttggctgggtttttccttgttcgcaccggacacctccagtgaccagacggcaaggtttttatcccagtgtatattgtcgac

SEQ ID NO: 19 - RNA, 47 nt, synthetic construct
  source 1..47: mol_type = other RNA
aggaaataagagagaaaagaagagtaagaagaaatataagagccacc

SEQ ID NO: 20 - RNA, 93 nt, synthetic construct
  source 1..93: mol_type = other RNA
gctgccttctgcggggcttgccttctggccatgcccttcttctctcccttgcacctgtacctcttggtctttgaataaagcctgagtaggaag